(19) United States
(12) Patent Application Publication
(10) Pub. No.: US 2024/0131045 A1
(43) Pub. Date: Apr. 25, 2024

(54) METHODS FOR TREATMENT OF VIRAL INFECTIONS

(71) Applicant: Gilead Sciences, Inc., Foster City, CA (US)

(72) Inventors: Casey B. Davis, San Francisco, CA (US); Rita Humeniuk, Belmont, CA (US); Abdul Naveed Shaik, Union City, CA (US)

(21) Appl. No.: 18/243,812

(22) Filed: Sep. 8, 2023

Related U.S. Application Data
(60) Provisional application No. 63/405,221, filed on Sep. 9, 2022.
(60) Provisional application No. 63/494,194, filed on Apr. 4, 2023.
(60) Provisional application No. 63/465,989, filed on May 12, 2023.

(51) Int. Cl. A61K 31/706

(52) U.S. Cl. CPC A61K 31/706

(57) ABSTRACT

The present disclosure relates to methods for treating viral infections in a patient.

### CROSS-REFERENCES TO RELATED APPLICATIONS

This application claims the benefit of U.S. Provisional Application No. 63/405,221, filed on Sep. 9, 2022, U.S. Provisional Application No. 63/494,194, filed on Apr. 4, 2023, and U.S. Provisional Application No. 63/465,989, filed on May 12, 2023. The entire contents of these applications are incorporated herein by reference in their entirety.

### FIELD OF THE INVENTION

The present disclosure relates to methods for treating viral infections.

### BACKGROUND

There is a need for compounds and methods for treating viral infections, for example paramyxoviridae, pneumoviridae, picornraviridae, flaviviridae, filoviridae, arenaviridae, orthomyxovirus, and coronaviridae infections.

### SUMMARY

Provided herein is a method of treating a viral infection in a patient in need thereof, wherein the method comprises administering to the patient a compound which is:

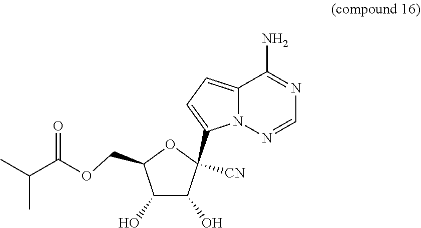 [SMILES: CC(C)C(=O)OC[C@H]1O[C@@](C#N)(c2ccc3c(N)ncnn23)[C@H](O)[C@@H]1O]

a deuterated compound thereof, or a pharmaceutically acceptable salt thereof, wherein compound 16, a deuterated compound thereof, or a pharmaceutically acceptable salt thereof, is administered in a dosage of less than 1,600 mg/dose.

Also provided herein is a method of treating a viral infection in a patient in need thereof, wherein the method comprises administering to the patient a compound which is compound 16, a deuterated compound thereof, or a pharmaceutically acceptable salt thereof, wherein the administering comprises determining an eGFR of the patient, selecting a standard dosage if the eGFR is at least 60 mL/min/1.73 m², and selecting an adjusted dosage that is less than the standard dosage if the eGFR is less than 60 mL/min/1.73 m².

Also provided herein is a method of treating a viral infection in a patient in need thereof, wherein the method comprises administering to the patient a compound of Formula A:

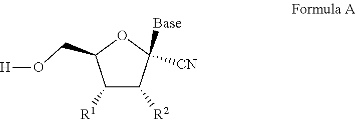

a deuterated compound of Formula A, a prodrug of the compound of Formula A, a prodrug of the deuterated compound of Formula A, or a pharmaceutically acceptable salt thereof, wherein R¹, R², and Base are defined herein, wherein when the prodrug of the compound of Formula A, the prodrug of the deuterated compound of Formula A, or the pharmaceutically acceptable salt thereof is administered, the prodrug of the compound of Formula A, the prodrug of the deuterated compound of Formula A, or the pharmaceutically acceptable salt thereof is converted substantially to the compound of Formula A or the deuterated compound of Formula A, and wherein the administration results in a mean Cₘₐₓ of the compound of Formula A, or the deuterated compound of Formula A, of less than 7,000 ng/mL.

### BRIEF DESCRIPTION OF DRAWINGS

FIG. 1 is a set of plots of PK exposures of compound 1 for dosing regimens of compound 16 including 350 mg BID, 700 mg loading with 350 mg BID maintenance, 500 mg BID, and 700 mg QD. Plotting was conducted using nonparametric superpositioning.

FIG. 2 is a set of plots comparing PK exposures of compound 1 for a 350 mg BID dosing regimen of compound 16 in patients with normal renal function (eGFR ≥90 mL/min/1.73 m²) and in patients with mild renal impairment (eGFR 60-89 mL/min/1.73 m²).

FIG. 3 is a graph showing agreement between PK exposure data for compound 1 after administration of compound 16, and a population-PK model described herein.

FIG. 4 is a plot comparing renal clearance values of compound 1 after administration of remdesivir and compound 16.

FIG. 5 is a plot showing a target exposure range of compound 1, based on simulated PK exposures after administration of 350 mg BID of compound 16 to patients with normal renal function (eGFR ≥90 mL/min/1.73 m²).

FIG. 6 is a plot comparing simulated PK exposures of compound 1 after administration of 350 mg BID of compound 16 to patients with normal renal function (eGFR ≥90 mL/min/1.73 m²) and patients with mild renal impairment (eGFR 60-89 mL/min/1.73 m²) with a target exposure range of compound 1.

FIG. 7 is a plot comparing simulated PK exposures of compound 1 after administration of 350 mg QD of compound 16 in patients with normal renal function (eGFR ≥90 mL/min/1.73 m²), patients with mild renal impairment (eGFR 60-89 mL/min/1.73 m²), and patients with moderate renal impairment (eGFR 30-59 mL/min/1.73 m²) with a target exposure range of compound 1.

FIG. 8 is a plot showing the proportion of simulated PK exposures within a target exposure range of compound 1 for dosing regimens of compound 16 including 350 mg BID, 350 mg QD, 350 mg QOD, and 350 mg loading with 150 mg QD maintenance, in patients with severe renal impairment (eGFR 15-29 mL/min/1.73 m²).

FIG. 9A is a schematic for single-dose cohorts.

FIG. 9B is a schematic for multiple-dose cohorts.

FIG. 10A is a plot showing plasma concentration-time profiles of the compound 1 metabolite in healthy participants receiving compound 16 in for single-dose cohorts.

FIG. 10B is a plot showing plasma concentration-time profiles of the compound 1 metabolite in healthy participants receiving compound 16 in for multiple-dose cohorts.

FIG. 10C is a plot showing plasma concentration-time profiles of the compound 1 metabolite in healthy participants receiving compound 16 in for food-effect cohorts.

FIG. 11 is a plot showing regression analysis of plasma exposure of compound 1 vs. eGFR following IV administration of remdesivir in participants with varying levels of renal impairment and matched controls.

FIG. 12 is a plot showing quadratic relation between eGFR and renal clearance (CLr) of compound 1 following IV administration of remdesivir in participants with varying levels of renal impairment.

FIG. 13A is a plot showing the compound 1 predicted steady-state exposures following a fixed dose of 350 mg BID in participants with normal renal function and those with varying degrees of RI. The predicted 5th to 95th percentile of compound 1 exposures following 350 mg BID oral doses of compound 16 in participants with normal renal function was considered the Target Exposure Range.

FIG. 13B is plot showing the compound 1 predicted steady-state exposures following the recommended adjusted dosing regimen for each RI population. The predicted 5th to 95th percentile of compound 1 exposures following 350 mg BID oral doses of compound 16 in participants with normal renal function was considered the Target Exposure Range.

### DETAILED DESCRIPTION

Unless stated otherwise, the following terms and phrases as used herein are intended to have the following meanings:

“Alkyl” refers to an unbranched or branched saturated hydrocarbon chain. For example, an alkyl group can have 1 to 20 carbon atoms (i.e., C₁-C₂₀ alkyl), 1 to 8 carbon atoms (i.e., C₁-C₈ alkyl), 1 to 6 carbon atoms (i.e., C₁-C₆ alkyl), or 1 to 3 carbon atoms (i.e., C₁-C₃ alkyl). Examples of suitable alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, 1-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), and 3,3-dimethyl-2-butyl (—CH(CH₃)C(CH₃)₃.

“Alkenyl” refers to an aliphatic group containing at least one carbon-carbon double bond and having from 2 to 20 carbon atoms (i.e., C₂₋₂₀ alkenyl), 2 to 8 carbon atoms (i.e., C₂₋₈ alkenyl), 2 to 6 carbon atoms (i.e., C₂₋₆ alkenyl), or 2 to 4 carbon atoms (i.e., C₂₋₄ alkenyl). Examples of alkenyl groups include ethenyl, propenyl, butadienyl (including 1,2-butadienyl and 1,3-butadienyl).

“Alkynyl” refers to an aliphatic group containing at least one carbon-carbon triple bond and having from 2 to 20 carbon atoms (i.e., C₂₋₂₀ alkynyl), 2 to 8 carbon atoms (i.e., C₂₋₈ alkynyl), 2 to 6 carbon atoms (i.e., C₂₋₆ alkynyl), or 2 to 4 carbon atoms (i.e., C₂₋₄ alkynyl). The term “alkynyl” also includes those groups having one triple bond and one double bond.

“Haloalkyl” is an alkyl group, as defined above, in which one or more hydrogen atoms of the alkyl group is replaced with a halogen atom. The alkyl portion of a haloalkyl group can have 1 to 20 carbon atoms (i.e., C₁-C₂₀ haloalkyl), 1 to 12 carbon atoms (i.e., C₁-C₁₂ haloalkyl), 1 to 8 carbon atoms (i.e., C₁-C₈ haloalkyl), 1 to 6 carbon atoms (i.e., C₁-C₆ alkyl) or 1 to 3 carbon atoms (i.e., C₁-C₃ alkyl). Examples of suitable haloalkyl groups include, but are not limited to, —CF₃, —CHF₂, —CFH₂, —CH₂CF₃, and the like.

“Aryl” means an aromatic hydrocarbon radical derived by the removal of one hydrogen atom from a single carbon atom of a parent aromatic ring system. For example, an aryl group can have 6 to 20 carbon atoms, 6 to 14 carbon atoms, or 6 to 10 carbon atoms. Typical aryl groups include, but are not limited to, radicals derived from benzene (e.g., phenyl), substituted benzene, naphthalene, anthracene, biphenyl, and the like.

“Heteroaryl” refers to an aromatic group having a single ring, multiple rings, or multiple fused rings, with one or more ring heteroatoms independently selected from nitrogen, oxygen, and sulfur. As used herein, heteroaryl includes 1 to 20 ring atoms (i.e., 1 to 20 membered heteroaryl), 3 to 12 ring atoms (i.e., 3 to 12 membered heteroaryl) or 3 to 8 carbon ring atoms (3 to 8 membered heteroaryl) or 5 to 6 ring atoms (5 to 6 membered heteroaryl). Examples of heteroaryl groups include pyrimidinyl, purinyl, pyridyl, pyridazinyl, benzothiazolyl, and pyrazolyl. Heteroaryl does not encompass or overlap with aryl as defined above.

“Carbocyclyl” or “carbocyclic ring” refers to a non-aromatic hydrocarbon ring consisting of carbon and hydrogen atoms, having from three to twenty carbon atoms, in certain embodiments having from three to fifteen carbon atoms, in certain embodiments having from three to ten carbon atoms, from three to eight carbon atoms, from three to seven carbon atoms, or from 3 to 6 carbon atoms and which is saturated or partially unsaturated and attached to the rest of the molecule by a single bond. Carbocyclic rings include, for example, cyclopropane, cyclobutane, cyclopentane, cyclopentene, cyclohexane, cyclohexene, 1,3-cyclohexadiene, 1,4-cyclohexadiene, cycloheptane, cycloheptene, and cyclooctane. Carbocyclic rings include cycloalkyl groups.

“Cycloalkyl” refers to a saturated cyclic alkyl group having a single ring or multiple rings including fused, bridged, and spiro ring systems. As used herein, cycloalkyl has from 3 to 20 ring carbon atoms (i.e., C₃₋₂₀ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Examples of cycloalkyl groups include cyclopropyl, cyclobutyl, cyclopentyl, cyclohexyl, cycloheptyl, and cyclooctyl.

“Heterocycle” or “heterocyclyl” as used herein includes by way of example and not limitation those heterocycles described in Paquette, Leo A.: Principles of Modern Heterocyclic Chemistry (W. A. Benjamin, New York, 1968), particularly Chapters 1, 3, 4, 6, 7, and 9; “The Chemistry of Heterocyclic Compounds, A Series of Monographs” (John Wiley & Sons, New York, 1950 to present), in particular Volumes 13, 14, 16, 19, and 28; and J. Am. Chem. Soc. (1960) 82:5566. For example, “heterocycle” includes a “carbocycle” as defined herein, wherein one or more (e.g. 1, 2, 3, or 4) carbon atoms have been replaced with a heteroatom (e.g. O, N, or S). As used herein, heterocycle or heterocyclyl has from 3 to 20 ring atoms, 3 to 12 ring atoms, 3 to 10 ring atoms, 3 to 8 ring atoms, or 3 to 6 ring atoms. The terms “heterocycle” or “heterocyclyl” includes saturated rings and partially unsaturated rings. Substituted heterocyclyls include, for example, heterocyclic rings substituted with any of the substituents described herein including carbonyl groups. A non-limiting example of a carbonyl substituted heterocyclyl is:

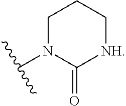 [SMILES: *N1CCCNC1=O]

Example heterocycles include, but are not limited to, tetrahydrofuranyl azetidinyl, and 2-oxo-1,3-dioxol-4-yl.

The term “optionally substituted” in reference to a particular moiety of a compound described herein such as the compound of Formula A or Formula I (e.g., an optionally substituted aryl group) refers to a moiety wherein all substituents are hydrogen or wherein one or more of the hydrogens of the moiety may be replaced by the listed substituents.

Unless otherwise specified, the carbon atoms of the compounds of Formula and Formula I are intended to have a valence of four. If in some chemical structure representations, carbon atoms do not have a sufficient number of variables attached to produce a valence of four, the remaining carbon substituents needed to provide a valence of four should be assumed to be hydrogen.

Any reference to the compounds described herein also includes a reference to a pharmaceutically acceptable salt thereof. Examples of pharmaceutically acceptable salts of the compounds described herein include salts derived from an appropriate base, such as an alkali metal or an alkaline earth (for example, Na⁺, Li⁺, K⁺, Ca⁺² and Mg⁺²), ammonium and NR₄⁺ (wherein R is defined herein). Pharmaceutically acceptable salts of a nitrogen atom or an amino group include (a) acid addition salts formed with inorganic acids, for example, hydrochloric acid, hydrobromic acid, sulfuric acid, sulfamic acids, phosphoric acid, nitric acid and the like; (b) salts formed with organic acids such as, for example, acetic acid, oxalic acid, tartaric acid, succinic acid, maleic acid, fumaric acid, gluconic acid, citric acid, malic acid, ascorbic acid, benzoic acid, isethionic acid, lactobionic acid, tannic acid, palmitic acid, alginic acid, polyglutamic acid, naphthalenesulfonic acid, methanesulfonic acid, p-toluenesulfonic acid, benzenesulfonic acid, naphthalenedisulfonic acid, polygalacturonic acid, malonic acid, sulfosalicylic acid, glycolic acid, 2-hydroxy-3-naphthoate, pamoate, salicylic acid, stearic acid, phthalic acid, mandelic acid, lactic acid, ethanesulfonic acid, lysine, arginine, glutamic acid, glycine, serine, threonine, alanine, isoleucine, leucine and the like; and (c) salts formed from elemental anions for example, chlorine, bromine, and iodine. Pharmaceutically acceptable salts of a compound of a hydroxy group include the anion of said compound in combination with a suitable cation such as Na and NR₄⁺. In some embodiments, R₄ is H, (C₁-C₈) alkyl, (C₂-C₈)alkenyl, (C₂-C₈) alkynyl, C₆-C₂₀ aryl, or C₂-C₂₀ heterocyclyl.

For therapeutic use, salts of active ingredients of the compounds described herein will be pharmaceutically acceptable, i.e., they will be salts derived from a pharmaceutically acceptable acid or base. It is also to be understood that the compositions herein comprise compounds described herein in their un-ionized, as well as zwitterionic form, and combinations with stoichiometric amounts of water as in hydrates. It is to be noted that all enantiomers, diastereomers, racemic mixtures, tautomers, polymorphs, and pseudopolymorphs of compounds described herein (e.g., compounds within the scope of Formula A or Formula I) and pharmaceutically acceptable salts thereof are embraced by the present disclosure. All mixtures of such enantiomers and diastereomers are within the scope of the present disclosure.

The compounds described herein may have chiral centers, e.g., chiral carbon or phosphorus atoms. The compounds described herein thus include racemic mixtures of all stereoisomers, including enantiomers, diastereomers, and atropisomers. In addition, the compounds described herein include enriched or resolved optical isomers at any or all asymmetric, chiral atoms. In other words, the chiral centers apparent from the depictions are provided as the chiral isomers or racemic mixtures. Both racemic and diastereomeric mixtures, as well as the individual optical isomers isolated or synthesized, substantially free of their enantiomeric or diastereomeric partners, are all within the scope of the disclosure. The racemic mixtures are separated into their individual, substantially optically pure isomers through appropriate techniques such as, for example, the separation of diastereomeric salts formed with optically active adjuncts, e.g., acids or bases followed by conversion back to the optically active substances. In most instances, the desired optical isomer is synthesized by means of stereospecific reactions, beginning with the appropriate stereoisomer of the desired starting material.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and 1, D and L, or (+) and (−) are employed to designate the sign of rotation of plane-polarized light by the compound, with S, (−), or 1 meaning that the compound is levorotatory while a compound prefixed with R, (+), or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

The compounds described herein may also exist as tautomeric isomers in certain cases. Although only one delocalized resonance structure may be depicted, all such forms are contemplated within the scope of the invention. For example, ene-amine tautomers can exist for purine, pyrimidine, imidazole, guanidine, amidine, and tetrazole systems and all their possible tautomeric forms are within the scope of the invention.

Any formula or structure described herein, including Formula A and Formula I compounds, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine and chlorine, such as, but not limited to ²H (deuterium, D), ³H (tritium), ¹¹C, ¹³C, ¹⁴C, ¹⁵N, ¹⁸F, P, ³²P, ³⁵S, ³⁶Cl and ¹²⁵I. Various isotopically labeled compounds of the present disclosure, for example those into which radioactive isotopes such as ³H, ³C and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The disclosure also includes compounds (e.g., compounds of Formula A or Formula I) in which from 1 to x hydrogens attached to a carbon atom is/are replaced by deuterium, in which x is the number of hydrogens in the molecule. Such compounds exhibit increased resistance to metabolism and are thus useful for increasing the half-life of any compound described herein (e.g., compounds of Formula A or Formula I) when administered to a mammal, particularly a human. See, for example, Foster, “Deuterium Isotope Effects in Studies of Drug Metabolism”, Trends Pharmacol. Sci. 5(12):524-527 (1984). In view of the present disclosure, such compounds are synthesized by means known in the art, for example by employing starting materials in which one or more hydrogens have been replaced by deuterium.

Deuterium labeled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F labeled compound may be useful for PET or SPECT studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in the compounds described herein.

For example, in the deuterated compound of Formula A, one or more hydrogen atoms attached to one or more carbon atoms of Formula A are replaced by deuterium. In some embodiments of the deuterated compound of Formula A, one hydrogen atom attached to one carbon atom of Formula A is replaced by deuterium. In some embodiments of the deuterated compound of Formula A, two hydrogen atoms attached to one carbon atom of Formula A are replaced by deuterium. In some embodiments of the deuterated compound of Formula A, two or more hydrogen atoms attached to two carbon atoms of Formula A are replaced by deuterium.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

Whenever a compound described herein is substituted with more than one of the same designated group, e.g., “R” or “R”, then it will be understood that the groups may be the same or different, i.e., each group is independently selected.

Wavy lines, [symbol] , indicate the site of covalent bond attachments to the adjoining substructures, groups, moieties, or atoms.

The term “treating”, as used herein, unless otherwise indicated, means reversing, alleviating, or inhibiting the progress of the disorder or condition to which such term applies, or one or more symptoms of such disorder or condition. The term “treatment”, as used herein, refers to the act of treating, as “treating” is defined immediately above.

“Prevention” or “preventing” means any treatment of a disease or condition that causes the clinical symptoms of the disease or condition not to develop. The compounds and compositions described herein may, in some embodiments, be administered to a subject (including a human) who is at risk of having the disease or condition. As used herein, the terms “preventing” and “prevention” encompass the administration of a compound, composition, or pharmaceutically acceptable salt according to the embodiments described herein pre- or post-exposure of the individual to a virus, but before the appearance of symptoms of the viral infection, and/or prior to the detection of the virus in the blood. The terms also refer to prevention of the appearance of symptoms of the disease and/or to prevent the virus from reaching detectible levels in the blood. The terms include both pre-exposure prophylaxis (PrEP), as well as post-exposure prophylaxis (PEP) and event driven or “on demand” prophylaxis. The terms also refer to prevention of perinatal transmission of a virus from mother to baby, by administration to the mother before giving birth and to the child within the first days of life. The terms also refer to prevention of transmission of a virus through blood transfusion.

The term “therapeutically effective amount”, as used herein, is the amount of a compound described herein (e.g., a compound of Formula A or Formula I) present in a composition described herein that is needed to provide a desired level of drug in the secretions and tissues of the airways and lungs, or alternatively, in the bloodstream of a subject to be treated to give an anticipated physiological response or desired biological effect when such a composition is administered by the chosen route of administration. The precise amount will depend upon numerous factors, for example the particular compound described herein (e.g., the compound of Formula A or Formula I), the specific activity of the composition, the delivery device employed, the physical characteristics of the composition, its intended use, as well as patient considerations such as severity of the disease state, patient cooperation, etc., and can readily be determined by one skilled in the art based upon the information provided herein.

The term “prodrug,” as used herein, refers to a biologically inactive derivative of a drug that, upon administration to the patient, can be converted to a parent drug according to some chemical or enzymatic pathway. For example, when a prodrug of the compound of Formula A, or a pharmaceutically acceptable salt thereof, is administered, the prodrug, or the pharmaceutically acceptable salt thereof, can be converted to the compound of Formula A. In another example, when a prodrug of the deuterated compound of Formula A, or a pharmaceutically acceptable salt thereof, is administered, the prodrug, or the pharmaceutically acceptable salt thereof, can be converted to the deuterated compound of Formula A.

The term “converted substantially,” as used herein in reference to a prodrug, refers to conversion of greater than 50% of a prodrug (e.g., a prodrug of the compound of Formula A, or a prodrug of the deuterated compound of Formula A) to a parent compound (e.g., the compound of Formula A, or the deuterated compound of Formula A). For example, the term “converted substantially” can refer to conversion of greater than 50%, greater than 60%, greater than 70%, greater than 80%, greater than 90%, greater than 95%, or greater than 99% of the prodrug of the compound of Formula A, the prodrug of the deuterated compound of Formula A, or the pharmaceutically acceptable salt thereof, to the compound of Formula A or the deuterated compound of Formula A.

The term “estimated glomerular filtration rate,” or “eGFR,” refers to an estimate of the glomerular filtration rate of a patient. Any suitable method for determining eGFR can be used. For example, eGFR can be calculated using the Cockcroft-Gault (CG) equation, the Modification of Diet in Renal Disease (MDRD) equation, the MDRD II equation, the Mayo Quadratic (Mayo) equation, and the Chronic Kidney Disease Epidemiology Collaboration (CKD-EPI) equation. In some embodiments, eGFR values are determined using the Modification of Diet in Renal Disease (MDRD) formula. See, e.g., Levey et al., Ann. Intern. Med. 145(4):247-54 (2006).

As used herein, “normal renal function,” used in reference to a patient, means that the patient does not have renal impairment or chronic kidney disease (CKD). A patient can be classified as having normal renal function using known methods, such as determining an eGFR of the patient (e.g., using an equation suitable for the patient's population) and comparing the eGFR to suitable guidelines (e.g., current medical guidelines for a given eGFR calculation equation and/or patient population). For example, an eGFR of at least 90 mL/min/1.73 m² can indicate normal renal function.

As used herein, “mild renal impairment,” used in reference to a patient, means that the patient has mild kidney damage. Mild renal impairment can also be referred to as “mild CKD.” A patient can be classified as having mild renal impairment using known methods, such as determining an eGFR of the patient (e.g., using an equation suitable for the patient's population) and comparing the eGFR to suitable guidelines (e.g., current medical guidelines for a given eGFR calculation equation and/or patient population). For example, an eGFR of at least 60 mL/min/1.73 m² to less than 90 mL/min/1.73 m² can indicate mild renal impairment.

As used herein, “moderate renal impairment,” used in reference to a patient, means that the patient has moderate kidney damage. Moderate renal impairment can also be referred to as “moderate CKD.” A patient can be classified as having moderate renal impairment using known methods, such as determining an eGFR of the patient (e.g., using an equation suitable for the patient's population) and comparing the eGFR to suitable guidelines (e.g., current medical guidelines for a given eGFR calculation equation and/or patient population). For example, an eGFR of at least 30 mL/min/1.73 m² to less than 60 mL/min/1.73 m² can indicate moderate renal impairment.

As used herein, “severe renal impairment,” used in reference to a patient, means that the patient has severe kidney damage. Severe renal impairment can also be referred to as “severe CKD.” A patient can be classified as having severe renal impairment using known methods, such as determining an eGFR of the patient (e.g., using an equation suitable for the patient's population) and comparing the eGFR to suitable guidelines (e.g., current medical guidelines for a given eGFR calculation equation and/or patient population). For example, an eGFR of at least 15 mL/min/1.73 m² to less than 30 mL/min/1.73 m² can indicate severe renal impairment.

As used herein, “mean,” when preceding a pharmacokinetic value (e.g. mean Cₘₐₓ) represents the arithmetic mean value of the pharmacokinetic value taken from a population of patients unless otherwise specified.

As used herein, “Cₘₐₓ” means the maximum observed plasma concentration. Unless specifically described, Cₘₐₓ can be calculated using standard methods.

As used herein, “AUC” refers to the area under the plasma concentration-time curve, which is a measure of total bioavailability. Unless specifically described, AUC can be calculated using standard methods.

As used herein, the term “AUC₀₋₂₄,” refers to the area under the plasma concentration-time curve from 0 time to 24 hours. Unless specifically described, AUC₀₋₂₄ can be calculated using standard methods.

As used herein, and in the absence of a specific reference to a particular pharmaceutically acceptable salt of a compound described herein, any dosages (e.g., expressed in milligrams) should be taken as referring to the amount of the compound on a free base basis. For example, a reference to “administering to the patient a compound which is:

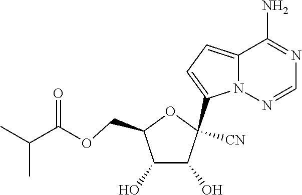 [SMILES: CC(C)C(=O)OC[C@H]1O[C@@](C#N)(c2ccc3c(N)ncnn23)[C@H](O)[C@@H]1O]

a deuterated compound thereof, or a pharmaceutically acceptable salt thereof . . . in a dosage of less than 1,600 mg/dose” means administration of an amount of the compound, the deuterated compound thereof, or the pharmaceutically salt thereof which provides the same amount of compound as 1600 mg of

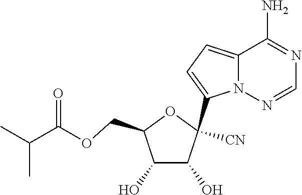 [SMILES: CC(C)C(=O)OC[C@H]1O[C@@](C#N)(c2ccc3c(N)ncnn23)[C@H](O)[C@@H]1O]

or the deuterated compound thereof, i.e., the free base compound.

Reference will now be made in detail to certain embodiments of the invention, examples of which are illustrated in the accompanying description. While the invention will be described in conjunction with the enumerated embodiments, it will be understood that they are not intended to limit the invention to those embodiments. On the contrary, the invention is intended to cover all alternatives, modifications, and equivalents, which may be included within the scope of the present invention.

Provided is a method of treating a viral infection in a patient in need thereof, wherein the method comprises administering to the patient a compound which is:

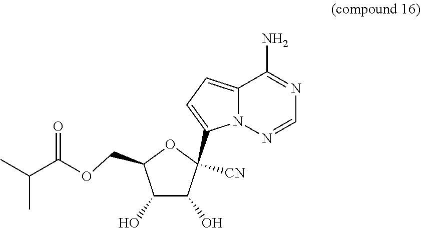 [SMILES: CC(C)C(=O)OC[C@H]1O[C@@](C#N)(c2ccc3c(N)ncnn23)[C@H](O)[C@@H]1O]

a deuterated compound thereof, or a pharmaceutically acceptable salt thereof, wherein compound 16, a deuterated compound thereof, or a pharmaceutically acceptable salt thereof, is administered in a dosage of less than 1,600 mg/dose.

In some embodiments, compound 16, a deuterated compound thereof, or a pharmaceutically acceptable salt thereof, is administered in a dosage of less than 1,500 mg/dose, less than 1,200 mg/dose, less than 1,000 mg/dose, less than 900 mg/dose, less than 800 mg/dose, less than 700 mg/dose, less than 600 mg/dose, or less than 500 mg/dose. In some embodiments, compound 16, a deuterated compound thereof, or a pharmaceutically acceptable salt thereof, is administered in a dosage of less than 900 mg/dose. In some embodiments, compound 16, a deuterated compound thereof, or a pharmaceutically acceptable salt thereof, is administered in a dosage of less than 600 mg/dose. In some embodiments, compound 16, a deuterated compound thereof, or a pharmaceutically acceptable salt thereof, is administered in a dosage of less than 500 mg/dose. In some embodiments, compound 16, a deuterated compound thereof, or a pharmaceutically acceptable salt thereof, is administered once daily or twice daily. In some embodiments, compound 16, a deuterated compound thereof, or a pharmaceutically acceptable salt thereof, is administered once daily. In some embodiments, compound 16, a deuterated compound thereof, or a pharmaceutically acceptable salt thereof, is administered twice daily.

In some embodiments, the patient has mild renal impairment. In some embodiments, the patient has an estimated glomerular filtration rate (eGFR) of at least 60 mL/min/1.73 m². In certain such embodiments, the method further includes determining that the patient has an eGFR of at least 60 mL/min/1.73 m² prior to the administration of compound 16, a deuterated compound thereof, or a pharmaceutically acceptable salt thereof. In some embodiments, the patient has an eGFR of 60 mL/min/1.73 m² to 89 mL/min/1.73 m². In certain such embodiments, the method further includes determining that the patient has an eGFR of 60 mL/min/1.73 m² to 89 mL/min/1.73 m² prior to the administration of compound 16, a deuterated compound thereof, or a pharmaceutically acceptable salt thereof.

In some embodiments, the patient has normal renal function. In some embodiments, the patient has an eGFR of at least 90 mL/min/1.73 m². In certain such embodiments, the method further includes determining that the patient has an eGFR of at least 90 mL/min/1.73 m² prior to the administration of compound 16, a deuterated compound thereof, or a pharmaceutically acceptable salt thereof. In some embodiments, the determining includes classifying the patient as potentially having an eGFR of less than 90 mL/min/1.73 m², and administering a blood test to the patient potentially having an eGFR of less than 90 mL/min/1.73 m².

In some embodiments, compound 16, a deuterated compound thereof, or a pharmaceutically acceptable salt thereof, is administered to the patient having an eGFR of least 60 mL/min/1.73 m² twice daily. In some embodiments, compound 16, a deuterated compound thereof, or a pharmaceutically acceptable salt thereof, is administered to the patient having an eGFR of least 60 mL/min/1.73 m² in a dosage of 100 mg/dose to 1,600 mg/dose, 100 mg/dose to 900 mg/dose, 100 mg/dose to 700 mg/dose, 100 mg/dose to 500 mg/dose, 100 mg/dose to 400 mg/dose, 100 mg/dose to 200 mg/dose, 200 mg/dose to 1,600 mg/dose, 200 mg/dose to 900 mg/dose, 200 mg/dose to 800 mg/dose, 200 mg/dose to 700 mg/dose, 200 mg/dose to 500 mg/dose, or 200 mg/dose to 400 mg/dose. In some embodiments, compound 16, a deuterated compound thereof, or a pharmaceutically acceptable salt thereof, is administered to the patient having an eGFR of least 60 mL/min/1.73 m² in a dosage of 250 mg/dose to 800 mg/dose. In some embodiments, compound 16, a deuterated compound thereof, or a pharmaceutically acceptable salt thereof, is administered to the patient having an eGFR of least 60 mL/min/1.73 m² in a dosage of 250 mg/dose to 500 mg/dose. In some embodiments, compound 16, a deuterated compound thereof, or a pharmaceutically acceptable salt thereof, is administered to the patient having an eGFR of least 60 mL/min/1.73 m² in a dosage of 350 mg/dose, 500 mg/dose, or 700 mg/dose. For example, in some embodiments, compound 16, a deuterated compound thereof, or a pharmaceutically acceptable salt thereof, is administered to the patient having an eGFR of least 60 mL/min/1.73 m² in a dosage of 350 mg/dose. In some embodiments, compound 16, a deuterated compound thereof, or a pharmaceutically acceptable salt thereof, is administered to the patient having an eGFR of least 60 mL/min/1.73 m² in a dosage of 500 mg/dose. In some embodiments, compound 16, a deuterated compound thereof, or a pharmaceutically acceptable salt thereof, is administered to the patient having an eGFR of least 60 mL/min/1.73 m² in a dosage of 700 mg/dose.

In some embodiments, the administration of compound 16, a deuterated compound thereof, or a pharmaceutically acceptable salt thereof, to the patient having an eGFR of at least 60 mL/min/1.73 m² results in a mean Cₘₐₓ of less than 7,000 ng/mL of

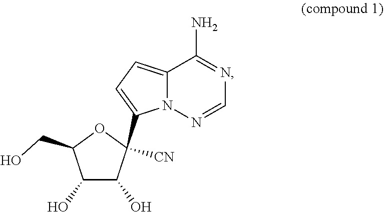 [SMILES: N#C[C@@]1(c2ccc3c(N)ncnn23)O[C@H](CO)[C@@H](O)[C@H]1O]

or a deuterated compound thereof.

In some embodiments, the administration of compound 16, a deuterated compound thereof, or a pharmaceutically acceptable salt thereof, to the patient having an eGFR of at least 60 mL/min/1.73 m² results in a mean Cₘₐₓ of 1,000 ng/mL to 6,500 ng/mL, 1,000 ng/mL to 6,000 ng/mL, 1,000 ng/mL to 5,500 ng/mL, 1,000 ng/mL to 5,000 ng/mL, 1,000 ng/mL to 4500 ng/mL, 2,000 ng/mL to 6,500 ng/mL, 2,000 ng/mL to 6,000 ng/mL, 2,000 ng/mL to 5,500 ng/mL, 2,000 ng/mL to 5,000 ng/mL, 2,000 ng/mL to 4500 ng/mL, 2,500 ng/mL to 6,500 ng/mL, 2,500 ng/mL to 6,000 ng/mL, 2,500 ng/mL to 5,500 ng/mL, 2,500 ng/mL to 5,000 ng/mL, or 2,500 ng/mL to 4500 ng/mL of compound 1, or a deuterated compound thereof. In some embodiments, the administration of compound 16, a deuterated compound thereof, or a pharmaceutically acceptable salt thereof, to the patient having an eGFR of at least 60 mL/min/1.73 m² results in a mean Cₘₐₓ of 2,000 ng/mL to 6,200 ng/mL of compound 1, or a deuterated compound thereof. In some embodiments, the administration of compound 16, a deuterated compound thereof, or a pharmaceutically acceptable salt thereof, to the patient having an eGFR of at least 60 mL/min/1.73 m² results in a mean Cₘₐₓ of 2,000 ng/mL to 4,900 ng/mL of compound 1, or a deuterated compound thereof.

In some embodiments, the administration of compound 16, a deuterated compound thereof, or a pharmaceutically acceptable salt thereof, to the patient having an eGFR of least 60 mL/min/1.73 m² results in a mean AUC₀₋₂₄ of less than 44,000 ng/mL*h of compound 1, or a deuterated compound thereof.

In some embodiments, the administration of compound 16, a deuterated compound thereof, or a pharmaceutically acceptable salt thereof, to the patient having an eGFR of least 60 mL/min/1.73 m² results in a mean AUC₀₋₂₄ of 12,000 ng/mL*h to 44,000 ng/mL*h, 12,000 ng/mL*h to 40,000 ng/mL*h, 12,000 ng/mL*h to 36,000 ng/mL*h, 12,000 ng/mL*h to 32,000 ng/mL*h, 16,000 ng/mL*h to 44,000 ng/mL*h, 16,000 ng/mL*h to 40,000 ng/mL*h, 16,000 ng/mL*h to 36,000 ng/mL*h, 16,000 ng/mL*h to 32,000 ng/mL*h, 20,000 ng/mL*h to 44,000 ng/mL*h, 20,000 ng/mL*h to 40,000 ng/mL*h, 20,000 ng/mL*h to 36,000 ng/mL*h, or 20,000 ng/mL*h to 32,000 ng/mL*h of compound 1, or a deuterated compound thereof. In some embodiments, the administration of compound 16, a deuterated compound thereof, or a pharmaceutically acceptable salt thereof, to the patient having an eGFR of least 60 mL/min/1.73 m² results in a mean AUC₀₋₂₄ of 18,200 ng/mL*h to 36,400 ng/mL*h of compound 1, or a deuterated compound thereof. In some embodiments, the administration of compound 16, a deuterated compound thereof, or a pharmaceutically acceptable salt thereof, to the patient having an eGFR of least 60 mL/min/1.73 m² results in a mean AUC₀₋₂₄ of 18,200 ng/mL*h to 32,000 ng/mL*h of compound 1, or a deuterated compound thereof.

In some embodiments, the patient has an eGFR of at least 60 mL/min/1.73 m², and compound 16, the deuterated compound, or a pharmaceutically acceptable salt thereof, is administered twice daily in a dosage of 350 mg/dose. In certain such embodiments, the administration results in a mean Cₘₐₓ of 2,000 ng/mL to 6,200 ng/mL and a mean AUC₀₋₂₄ of 18,200 ng/mL*h to 36,400 ng/mL*h of compound 1, or a deuterated compound thereof.

In some embodiments, the patient has normal renal function or mild kidney impairment, and compound 16, the deuterated compound, or a pharmaceutically acceptable salt thereof, is administered twice daily in a dosage of 350 mg/dose. In certain such embodiments, the administration results in a mean Cₘₐₓ of 2,000 ng/mL to 6,200 ng/mL and a mean AUC₀₋₂₄ of 18,200 ng/mL*h to 36,400 ng/mL*h of compound 1, or a deuterated compound thereof.

In some embodiments, the patient has normal renal function, and compound 16, the deuterated compound, or a pharmaceutically acceptable salt thereof, is administered twice daily in a dosage of 350 mg/dose. In certain such embodiments, the administration results in a mean Cₘₐₓ of 2,000 ng/mL to 6,200 ng/mL and a mean AUC₀₋₂₄ of 18,200 ng/mL*h to 36,400 ng/mL*h of compound 1, or a deuterated compound thereof.

In some embodiments, the patient has mild kidney impairment, and compound 16, the deuterated compound, or a pharmaceutically acceptable salt thereof, is administered twice daily in a dosage of 350 mg/dose. In certain such embodiments, the administration results in a mean Cₘₐₓ of 2,000 ng/mL to 6,200 ng/mL and a mean AUC₀₋₂₄ of 18,200 ng/mL*h to 36,400 ng/mL*h of compound 1, or a deuterated compound thereof.

In some embodiments, the patient has an eGFR of less than 60 mL/min/1.73 m². In some embodiments, the patient has moderate renal impairment. In some embodiments, the patient has an eGFR of 30 mL/min/1.73 m² to 59 mL/min/1.73 m². In certain such embodiments, the method further includes determining that the patient has an eGFR of 30 mL/min/1.73 m² to 59 mL/min/1.73 m² prior to the administration of compound 16, a deuterated compound thereof, or a pharmaceutically acceptable salt thereof. In some embodiments, the determining includes classifying the patient as potentially having an eGFR of less than 60 mL/min/1.73 m², and administering a blood test to the patient potentially having an eGFR of less than 60 mL/min/1.73 m².

In some embodiments, compound 16, a deuterated compound thereof, or a pharmaceutically acceptable salt thereof, is administered to the patient having an eGFR of 30 mL/min/1.73 m² to 59 mL/min/1.73 m² once daily. In some embodiments, compound 16, a deuterated compound thereof, or a pharmaceutically acceptable salt thereof, is administered to the patient having an eGFR of 30 mL/min/1.73 m² to 59 mL/min/1.73 m² in a dosage of 50 mg/dose to 900 mg/dose, 50 mg/dose to 700 mg/dose, 50 mg/dose to 500 mg/dose, 50 mg/dose to 400 mg/dose, 50 mg/dose to 200 mg/dose, 100 mg/dose to 900 mg/dose, 100 mg/dose to 800 mg/dose, 100 mg/dose to 700 mg/dose, 100 mg/dose to 500 mg/dose, 100 mg/dose to 400 mg/dose, or 100 mg/dose to 200 mg/dose. In some embodiments, compound 16, a deuterated compound thereof, or a pharmaceutically acceptable salt thereof, is administered to the patient having an eGFR of 30 mL/min/1.73 m² to 59 mL/min/1.73 m² in a dosage of 50 mg/dose to 650 mg/dose. In some embodiments, compound 16, a deuterated compound thereof, or a pharmaceutically acceptable salt thereof, is administered to the patient having an eGFR of 30 mL/min/1.73 m² to 59 mL/min/1.73 m² in a dosage of 150 mg/dose to 500 mg/dose. In some embodiments, compound 16, a deuterated compound thereof, or a pharmaceutically acceptable salt thereof, is administered to the patient having an eGFR of 30 mL/min/1.73 m² to 59 mL/min/1.73 m² in a dosage of 350 mg/dose.

In some embodiments, the administration of compound 16, a deuterated compound thereof, or a pharmaceutically acceptable salt thereof, to the patient having an eGFR of 30 mL/min/1.73 m² to 59 mL/min/1.73 m² results in a mean Cₘₐₓ of less than 7,000 ng/mL of compound 1, or a deuterated compound thereof.

In some embodiments, the administration of compound 16, a deuterated compound thereof, or a pharmaceutically acceptable salt thereof, to the patient having an eGFR of 30 mL/min/1.73 m² to 59 mL/min/1.73 m² results in a mean Cₘₐₓ of 1,000 ng/mL to 6,500 ng/mL, 1,000 ng/mL to 6,000 ng/mL, 1,000 ng/mL to 5,500 ng/mL, 1,000 ng/mL to 5,000 ng/mL, 1,000 ng/mL to 4500 ng/mL, 2,000 ng/mL to 6,500 ng/mL, 2,000 ng/mL to 6,000 ng/mL, 2,000 ng/mL to 5,500 ng/mL, 2,000 ng/mL to 5,000 ng/mL, 2,000 ng/mL to 4500 ng/mL, 2,500 ng/mL to 6,500 ng/mL, 2,500 ng/mL to 6,000 ng/mL, 2,500 ng/mL to 5,500 ng/mL, 2,500 ng/mL to 5,000 ng/mL, or 2,500 ng/mL to 4500 ng/mL of compound 1, or a deuterated compound thereof. In some embodiments, the administration of compound 16, a deuterated compound thereof, or a pharmaceutically acceptable salt thereof, to the patient having an eGFR of 30 mL/min/1.73 m² to 59 mL/min/1.73 m² results in a mean Cₘₐₓ of 2,000 ng/mL to 6,200 ng/mL of compound 1, or a deuterated compound thereof. In some embodiments, the administration of compound 16, a deuterated compound thereof, or a pharmaceutically acceptable salt thereof, to the patient having an eGFR of 30 mL/min/1.73 m² to 59 mL/min/1.73 m² results in a mean Cₘₐₓ of 2,000 ng/mL to 4,900 ng/mL of compound 1, or a deuterated compound thereof.

In some embodiments, the administration of compound 16, a deuterated compound thereof, or a pharmaceutically acceptable salt thereof, to the patient having an eGFR of 30 mL/min/1.73 m² to 59 mL/min/1.73 m² results in a mean AUC₀₋₂₄ of less than 44,000 ng/mL*h of compound 1, or a deuterated compound thereof.

In some embodiments, the administration of compound 16, a deuterated compound thereof, or a pharmaceutically acceptable salt thereof, to the patient having an eGFR of 30 mL/min/1.73 m² to 59 mL/min/1.73 m² results in a mean AUC₀₋₂₄ of 12,000 ng/mL*h to 44,000 ng/mL*h, 12,000 ng/mL*h to 40,000 ng/mL*h, 12,000 ng/mL*h to 36,000 ng/mL*h, 12,000 ng/mL*h to 32,000 ng/mL*h, 16,000 ng/mL*h to 44,000 ng/mL*h, 16,000 ng/mL*h to 40,000 ng/mL*h, 16,000 ng/mL*h to 36,000 ng/mL*h, 16,000 ng/mL*h to 32,000 ng/mL*h, 20,000 ng/mL*h to 44,000 ng/mL*h, 20,000 ng/mL*h to 40,000 ng/mL*h, 20,000 ng/mL*h to 36,000 ng/mL*h, or 20,000 ng/mL*h to 32,000 ng/mL*h of compound 1, or a deuterated compound thereof. In some embodiments, the administration of compound 16, a deuterated compound thereof, or a pharmaceutically acceptable salt thereof, to the patient having an eGFR of 30 mL/min/1.73 m² to 59 mL/min/1.73 m² results in a mean AUC₀₋₂₄ of 18,200 ng/mL*h to 36,400 ng/mL*h of compound 1, or a deuterated compound thereof. In some embodiments, the administration of compound 16, a deuterated compound thereof, or a pharmaceutically acceptable salt thereof, to the patient having an eGFR of 30 mL/min/1.73 m² to 59 mL/min/1.73 m² results in a mean AUC₀₋₂₄ of 18,200 ng/mL*h to 32,000 ng/mL*h of compound 1, or a deuterated compound thereof.

In some embodiments, the patient has an eGFR of 30 mL/min/1.73 m² to 59 mL/min/1.73 m², and compound 16, the deuterated compound, or a pharmaceutically acceptable salt thereof, is administered once daily in a dosage of 350 mg/dose. In certain such embodiments, the administration results in a mean Cₘₐₓ of 2,000 ng/mL to 6,200 ng/mL and a mean AUC₀₋₂₄ of 18,200 ng/mL*h to 36,400 ng/mL*h of compound 1, or a deuterated compound thereof.

In some embodiments, the patient has moderate renal impairment, and compound 16, the deuterated compound, or a pharmaceutically acceptable salt thereof, is administered once daily in a dosage of 350 mg/dose. In certain such embodiments, the administration results in a mean Cₘₐₓ of 2,000 ng/mL to 6,200 ng/mL and a mean AUC₀₋₂₄ of 18,200 ng/mL*h to 36,400 ng/mL*h of compound 1, or a deuterated compound thereof.

In some embodiments, the patient has severe renal impairment. In some embodiments, the patient has an eGFR of 15 mL/min/1.73 m² to 29 mL/min/1.73 m². In certain such embodiments, the method further includes determining that the patient has an eGFR of 15 mL/min/1.73 m² to 29 mL/min/1.73 m² prior to the administration of compound 16, a deuterated compound thereof, or a pharmaceutically acceptable salt thereof. In some embodiments, the determining includes classifying the patient as potentially having an eGFR of less than 60 mL/min/1.73 m², and administering a blood test to the patient potentially having an eGFR of less than 60 mL/min/1.73 m².

In some embodiments, compound 16, a deuterated compound thereof, or a pharmaceutically acceptable salt thereof, is administered to the patient having an eGFR of 15 mL/min/1.73 m² to 29 mL/min/1.73 m² once daily. In some embodiments, compound 16, a deuterated compound thereof, or a pharmaceutically acceptable salt thereof, is administered to the patient having an eGFR of 15 mL/min/1.73 m² to 29 mL/min/1.73 m² in a dosage of 50 mg/dose to 900 mg/dose, 50 mg/dose to 700 mg/dose, 50 mg/dose to 500 mg/dose, 50 mg/dose to 400 mg/dose, 50 mg/dose to 200 mg/dose, 100 mg/dose to 900 mg/dose, 100 mg/dose to 800 mg/dose, 100 mg/dose to 700 mg/dose, 100 mg/dose to 500 mg/dose, 100 mg/dose to 400 mg/dose, or 100 mg/dose to 200 mg/dose. In some embodiments, compound 16, a deuterated compound thereof, or a pharmaceutically acceptable salt thereof, is administered to the patient having an eGFR of 15 mL/min/1.73 m² to 29 mL/min/1.73 m² in a dosage of 50 mg/dose to 650 mg/dose, 50 mg/dose to 500 mg/dose, or 50 mg/dose to 400 mg/dose. In some embodiments, compound 16, the deuterated compound, or a pharmaceutically acceptable salt thereof, is administered to the patient having an eGFR of 15 mL/min/1.73 m² to 29 mL/min/1.73 m² in a dosage of 50 mg/dose to 350 mg/dose, or 150 mg/dose to 500 mg/dose. In some embodiments, compound 16, a deuterated compound thereof, or a pharmaceutically acceptable salt thereof, is administered to the patient having an eGFR of 15 mL/min/1.73 m² to 29 mL/min/1.73 m² in a dosage of 350 mg/dose. In some embodiments, compound 16, a deuterated compound thereof, or a pharmaceutically acceptable salt thereof, is administered to the patient having an eGFR of 15 mL/min/1.73 m² to 29 mL/min/1.73 m² in a dosage of 150 mg/dose. In some embodiments, compound 16, a deuterated compound thereof, or a pharmaceutically acceptable salt thereof, is administered to the patient having an eGFR of 15 mL/min/1.73 m² to 29 mL/min/1.73 m² in a dosage of 350 mg/dose on a first day of administration. In certain such embodiments, compound 16, a deuterated compound thereof, or a pharmaceutically acceptable salt thereof, is administered in a dosage of 150 mg/dose on each day of administration after the first day.

In some embodiments, compound 16, a deuterated compound thereof, or a pharmaceutically acceptable salt thereof, is administered to the patient having an eGFR of 15 mL/min/1.73 m² to 29 mL/min/1.73 m² in a dosage of 175 mg/dose.

In certain such embodiments, compound 16, a deuterated compound thereof, or a pharmaceutically acceptable salt thereof, is administered in a dosage of 175 mg/dose on each day of administration after the first day. In some embodiments, compound 16, a deuterated compound thereof, or a pharmaceutically acceptable salt thereof, is administered in a dosage of 175 mg/dose on each day 2, 3, 4, and 5 of administration after the first day.

In some embodiments, the administration of compound 16, a deuterated compound thereof, or a pharmaceutically acceptable salt thereof, to the patient having an eGFR of 15 mL/min/1.73 m² to 29 mL/min/1.73 m² results in a mean Cₘₐₓ of less than 7,000 ng/mL of compound 1, or a deuterated compound thereof.

In some embodiments, the administration of compound 16, a deuterated compound thereof, or a pharmaceutically acceptable salt thereof, to the patient having an eGFR of 15 mL/min/1.73 m² to 29 mL/min/1.73 m² results in a mean Cₘₐₓ of 1,000 ng/mL to 6,500 ng/mL, 1,000 ng/mL to 6,000 ng/mL, 1,000 ng/mL to 5,500 ng/mL, 1,000 ng/mL to 5,000 ng/mL, 1,000 ng/mL to 4500 ng/mL, 2,000 ng/mL to 6,500 ng/mL, 2,000 ng/mL to 6,000 ng/mL, 2,000 ng/mL to 5,500 ng/mL, 2,000 ng/mL to 5,000 ng/mL, 2,000 ng/mL to 4500 ng/mL, 2,500 ng/mL to 6,500 ng/mL, 2,500 ng/mL to 6,000 ng/mL, 2,500 ng/mL to 5,500 ng/mL, 2,500 ng/mL to 5,000 ng/mL, or 2,500 ng/mL to 4500 ng/mL of compound 1, or a deuterated compound thereof. In some embodiments, the administration of compound 16, a deuterated compound thereof, or a pharmaceutically acceptable salt thereof, to the patient having an eGFR of 15 mL/min/1.73 m² to 29 mL/min/1.73 m² results in a mean Cₘₐₓ of 2,000 ng/mL to 6,200 ng/mL of compound 1, or a deuterated compound thereof. In some embodiments, the administration of compound 16, a deuterated compound thereof, or a pharmaceutically acceptable salt thereof, to the patient having an eGFR of 15 mL/min/1.73 m² to 29 mL/min/1.73 m² results in a mean Cₘₐₓ of 2,000 ng/mL to 4,900 ng/mL of compound 1, or a deuterated compound thereof.

In some embodiments, the administration of compound 16, a deuterated compound thereof, or a pharmaceutically acceptable salt thereof, to the patient having an eGFR of 15 mL/min/1.73 m² to 29 mL/min/1.73 m² results in a mean AUC₀₋₂₄ of less than 44,000 ng/mL*h of compound 1, or a deuterated compound thereof.

In some embodiments, the administration of compound 16, a deuterated compound thereof, or a pharmaceutically acceptable salt thereof, to the patient having an eGFR of 15 mL/min/1.73 m² to 29 mL/min/1.73 m² results in a mean AUC₀₋₂₄ of 12,000 ng/mL*h to 44,000 ng/mL*h, 12,000 ng/mL*h to 40,000 ng/mL*h, 12,000 ng/mL*h to 36,000 ng/mL*h, 12,000 ng/mL*h to 32,000 ng/mL*h, 16,000 ng/mL*h to 44,000 ng/mL*h, 16,000 ng/mL*h to 40,000 ng/mL*h, 16,000 ng/mL*h to 36,000 ng/mL*h, 16,000 ng/mL*h to 32,000 ng/mL*h, 20,000 ng/mL*h to 44,000 ng/mL*h, 20,000 ng/mL*h to 40,000 ng/mL*h, 20,000 ng/mL*h to 36,000 ng/mL*h, or 20,000 ng/mL*h to 32,000 ng/mL*h of compound 1, or a deuterated compound thereof. In some embodiments, the administration of compound 16, a deuterated compound thereof, or a pharmaceutically acceptable salt thereof, to the patient having an eGFR of 15 mL/min/1.73 m² to 29 mL/min/1.73 m² results in a mean AUC₀₋₂₄ of 18,200 ng/mL*h to 36,400 ng/mL*h of compound 1, or a deuterated compound thereof. In some embodiments, the administration of compound 16, a deuterated compound thereof, or a pharmaceutically acceptable salt thereof, to the patient having an eGFR of 15 mL/min/1.73 m² to 29 mL/min/1.73 m² results in a mean AUC₀₋₂₄ of 18,200 ng/mL*h to 32,000 ng/mL*h of compound 1, or a deuterated compound thereof.

In some embodiments, the patient has an eGFR of 15 mL/min/1.73 m² to 29 mL/min/1.73 m², and compound 16, a deuterated compound thereof, or a pharmaceutically acceptable salt thereof, is administered once in a dosage of 350 mg/dose, on a first day of administration, and once daily in a dosage of 150 mg/dose on each day of administration after the first day. In certain such embodiments, the administration results in a mean Cₘₐₓ of 2,000 ng/mL to 6,200 ng/mL and a mean AUC₀₋₂₄ of 18,200 ng/mL*h to 36,400 ng/mL*h of compound 1, or a deuterated compound thereof.

In some embodiments, the patient has an eGFR of 15 mL/min/1.73 m² to 29 mL/min/1.73 M², and compound 16, a deuterated compound thereof, or a pharmaceutically acceptable salt thereof, is administered once in a dosage of 350 mg/dose, on a first day of administration, and once daily in a dosage of 175 mg/dose on each day of administration after the first day. In some embodiments, the patient has an eGFR of 15 mL/min/1.73 m² to 29 mL/min/1.73 m², and compound 16, a deuterated compound thereof, or a pharmaceutically acceptable salt thereof, is administered once in a dosage of 350 mg/dose, on a first day of administration, and once daily in a dosage of 175 mg/dose on each day 2, 3, 4, and 5 of administration after the first day.

In some embodiments, the patient has severe renal impairment, and compound 16, a deuterated compound thereof, or a pharmaceutically acceptable salt thereof, is administered once in a dosage of 350 mg/dose, on a first day of administration, and once daily in a dosage of 150 mg/dose on each day of administration after the first day. In certain such embodiments, the administration results in a mean Cₘₐₓ of 2,000 ng/mL to 6,200 ng/mL and a mean AUC₀₋₂₄ of 18,200 ng/mL*h to 36,400 ng/mL*h of compound 1, or a deuterated compound thereof.

In some embodiments, the patient has severe renal impairment, and compound 16, a deuterated compound thereof, or a pharmaceutically acceptable salt thereof, is administered once in a dosage of 350 mg/dose, on a first day of administration, and once daily in a dosage of 175 mg/dose on each day of administration after the first day. In some embodiments, the patient has severe renal impairment, and compound 16, a deuterated compound thereof, or a pharmaceutically acceptable salt thereof, is administered once in a dosage of 350 mg/dose, on a first day of administration, and once daily in a dosage of 175 mg/dose on each day 2, 3, 4, and 5 of administration after the first day.

In some embodiments, compound 16, a deuterated compound thereof, or a pharmaceutically acceptable salt thereof, is administered orally. In some embodiments, compound 16, a deuterated compound thereof, or a pharmaceutically acceptable salt thereof, is administered for three consecutive days, or for five consecutive days. For example, in some embodiments, compound 16, a deuterated compound thereof, or a pharmaceutically acceptable salt thereof, is administered for five consecutive days. In some embodiments, compound 16, a deuterated compound thereof, or a pharmaceutically acceptable salt thereof, is administered for three consecutive days.

Also provided herein is a method for treating a viral infection in a patient in need thereof, wherein the method comprises administering to the patient a compound which is compound 16, a deuterated compound thereof, or a pharmaceutically acceptable salt thereof, wherein the administering comprises determining an eGFR of the patient, selecting a standard dosage if the eGFR is at least 60 mL/min/1.73 m², and selecting an adjusted dosage that is less than the standard dosage if the eGFR is less than 60 mL/min/1.73 m².

In some embodiments, the standard dosage is 100 mg/dose to 1,600 mg/dose, 100 mg/dose to 900 mg/dose, 100 mg/dose to 700 mg/dose, 100 mg/dose to 500 mg/dose, 100 mg/dose to 400 mg/dose, 100 mg/dose to 200 mg/dose, 200 mg/dose to 1,600 mg/dose, 200 mg/dose to 900 mg/dose, 200 mg/dose to 800 mg/dose, 200 mg/dose to 700 mg/dose, 200 mg/dose to 500 mg/dose, or 200 mg/dose to 400 mg/dose. In some embodiments, the standard dosage is 250 mg/dose to 800 mg/dose. In some embodiments, the standard dosage is 350 mg/dose, 500 mg/dose, or 700 mg/dose. In some embodiments, the standard dosage is 700 mg/dose. In some embodiments, the standard dosage is 500 mg/dose. In some embodiments, the standard dosage is 350 mg/dose. In some embodiments, the standard dosage is administered twice daily. In some embodiments, the standard dosage is administered once daily.

In some embodiments, the adjusted dosage is 50 mg/dose to 900 mg/dose, 50 mg/dose to 700 mg/dose, 50 mg/dose to 500 mg/dose, 50 mg/dose to 400 mg/dose, 50 mg/dose to 200 mg/dose, 100 mg/dose to 900 mg/dose, 100 mg/dose to 800 mg/dose, 100 mg/dose to 700 mg/dose, 100 mg/dose to 500 mg/dose, 100 mg/dose to 400 mg/dose, or 100 mg/dose to 200 mg/dose. In some embodiments, the adjusted dosage is 50 mg/dose to 650 mg/dose. In some embodiments, the adjusted dosage is 350 mg/dose. In some embodiments, the adjusted dosage is 150 mg/dose. In some embodiments, the adjusted dosage is 350 mg/dose on a first day of administration, and 150 mg/dose on each day of administration after the first day. In some embodiments, the adjusted dosage is administered once daily.

In some embodiments, the adjusted dosage is 175 mg/dose.

In some embodiments, the adjusted dosage is 350 mg/dose on a first day of administration, and 175 mg/dose on each day of administration after the first day. In some embodiments, the adjusted dosage is 350 mg/dose on a first day of administration, and 175 mg/dose on each day of administration after the first day. In some embodiments, the adjusted dosage is 350 mg/dose on a first day of administration, and 175 mg/dose on each day of administration after the first day. In some embodiments, the adjusted dosage is 350 mg/dose on a first day of administration, and 175 mg/dose on each day 2, 3, 4, and 5 of administration after the first day.

In some embodiments, a deuterated compound of compound 16 is administered. In certain such embodiments, the deuterated compound is

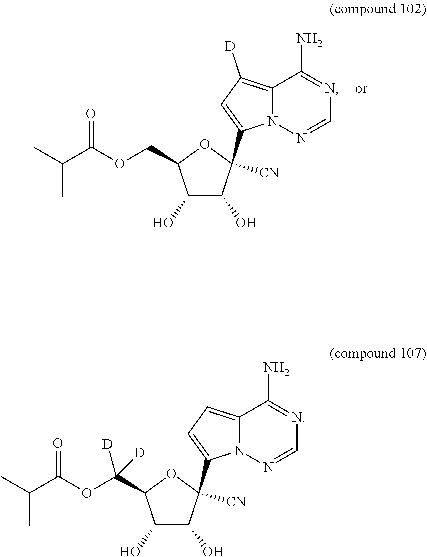 [SMILES: [2H]c1cc([C@]2(C#N)O[C@H](COC(=O)C(C)C)[C@@H](O)[C@H]2O)n2ncnc(N)c12 ; [2H]C([2H])(OC(=O)C(C)C)[C@H]1O[C@@](C#N)(c2ccc3c(N)ncnn23)[C@H](O)[C@@H]1O]

In some embodiments, a pharmaceutically acceptable salt of compound 16 is administered.

In some embodiments, compound 16 is administered.

Also provided herein is a compound which is compound 16, a deuterated compound thereof, or a pharmaceutically acceptable salt thereof, for use in a method of treating a viral infection in a patient in need thereof, wherein the method comprises administering compound 16, a deuterated compound thereof, or a pharmaceutically acceptable salt thereof, to the patient in a dosage of less than 1,600 mg/dose.

Also provided herein is the use of a compound which is compound 16, a deuterated compound thereof, or a pharmaceutically acceptable salt thereof, for the manufacture of a medicament for the treatment of a viral infection in a patient in need thereof, wherein the treatment comprises administering compound 16, a deuterated compound thereof, or a pharmaceutically acceptable salt thereof, to the patient in a dosage of less than 1,600 mg/dose.

Also provided herein is a compound which is compound 16, a deuterated compound thereof, or a pharmaceutically acceptable salt thereof, for use in a method for treating a viral infection in a patient in need thereof, wherein the method comprises determining an eGFR of the patient, selecting a standard dosage of compound 16, a deuterated compound thereof, or a pharmaceutically acceptable salt thereof, if the eGFR is at least 60 mL/min/1.73 m², and selecting an adjusted dosage of compound 16, a deuterated compound thereof, or a pharmaceutically acceptable salt thereof, that is less than the standard dosage if the eGFR is less than 60 mL/min/1.73 m².

Also provided herein is the use of a compound which is compound 16, a deuterated compound thereof, or a pharmaceutically acceptable salt thereof, for the manufacture of a medicament for the treatment of a viral infection in a patient in need thereof, wherein the treatment comprises determining an eGFR of the patient, selecting a standard dosage of compound 16, a deuterated compound thereof, or a pharmaceutically acceptable salt thereof, if the eGFR is at least 60 mL/min/1.73 m², and selecting an adjusted dosage of compound 16, a deuterated compound thereof, or a pharmaceutically acceptable salt thereof, that is less than the standard dosage if the eGFR is less than 60 mL/min/1.73 m².

Also provided herein is a method of treating a viral infection in a patient in need thereof, wherein the method comprises administering to the patient a compound of Formula A:

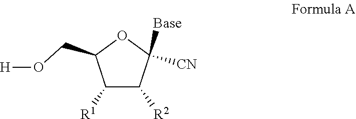

a deuterated compound of Formula A, a prodrug of the compound of Formula A, a prodrug of the deuterated compound of Formula A, or a pharmaceutically acceptable salt thereof, wherein:
R¹ is —OH, —OC(═O)R⁴, or —OC(═O)OR⁴:
R² is —OH, —OC(═O)R⁵, or —OC(═O)OR⁵; or
R¹ and R² are taken together to form —OC(═O)O—, —OP(═O)(OH)O—, or —OCHR⁶O—;
R⁴ and R⁵ are each independently H, C₁-C₈ alkyl, C₂-C₈ alkenyl, C₂-C₈ alkynyl, C₃-C₈ carbocyclyl, C₆-C₁₀ aryl, 4 to 6 membered heterocyclyl containing 1, 2, or 3 heteroatoms selected from N, O, and S, or 5 to 6 membered heteroaryl containing 1, 2, or 3 heteroatoms selected from N, O, and S; wherein each C₁-C₈ alkyl, C₂-C₈ alkenyl, C₂-C₈ alkynyl, C₃-C₈ carbocyclyl, C₆-C₁₀ aryl, 4 to 6 membered heterocyclyl, and 5 to 6 membered heteroaryl of R⁴ and R⁵ is independently, optionally substituted with one, two, or three R^{a} substituents;
R⁶ is H, C₁-C₆ alkyl, C₁-C₆ alkoxy, 5 to 6 membered heteroaryl containing 1, 2, or 3 heteroatoms selected from N, O, and S, or C₆-C₁₀ aryl; wherein each 5 to 6 membered heteroaryl and C₆-C₁₀ aryl of R⁶ is independently, optionally substituted with one, two, or three R^{b} substituents;
Base is

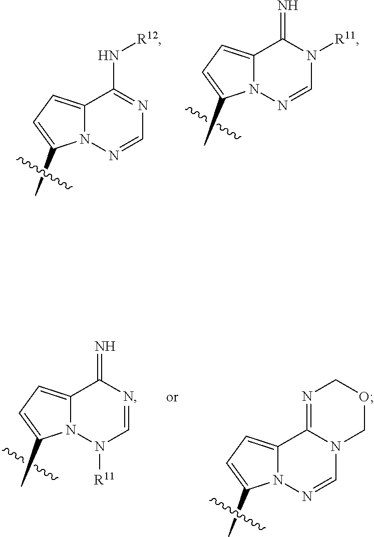

R¹¹ is C₁-C₆ alkyl optionally substituted with —OP(═O)(OH)(OR¹⁴);
R¹² is H, C₁-C₆ alkyl, —C(═O)R¹³ or —C(═O)OR¹³;
each R¹³ is independently H, C₁-C₁₀ alkyl, C₆-C₁₀ aryl, —O—C₆-C₁₀ aryl, or —O—C₁-C₁₀ alkyl; wherein each C₁-C₁₀ alkyl, C₆-C₁₀ aryl, —O—C₆-C₁₀ aryl, and —O—C₁-C₁₀ alkyl of R¹³ is independently, optionally substituted with one, two, or three R^{c} substituents;
each R^{a} is independently halo, cyano, C₁-C₆ alkyl, carbonyl, —N₃, —OR, —NR⁹R¹⁰, —OP(═O)(OH)₂, C₃-C₈ carbocyclyl, 4 to 6 membered heterocyclyl containing 1, 2, or 3 heteroatoms selected from N, O, and S, 5 to 6 membered heteroaryl containing 1, 2, or 3 heteroatoms selected from N, O, and S, or phenyl; wherein each 4 to 6 membered heterocyclyl of R^{a} is independently, optionally substituted with one, two, or three R^{d} substituents; wherein each phenyl of R is independently, optionally substituted with one, two or three R^{e} substituents;
R⁸, R⁹, and R¹⁰ are each independently H, C₁-C₆ alkyl, C₁-C₆ haloalkyl, or C₃-C₆ cycloalkyl
each R^{b} is independently halo, cyano, C₁-C₆ alkoxy, or C₁-C₆ alkyl;
each R^{c} is independently halo, cyano, —OP(═O)(OH)(OR¹⁴) or phenyl; wherein each phenyl of R^{c} is optionally substituted with —OP(═O)(OH)(OR⁴);
each R¹⁴ is independently H, C₁-C₈ alkyl, C₃-C₈ carbocyclyl, C₆-C₁₀ aryl, or 5 to 6 membered heteroaryl containing 1, 2, or 3 heteroatoms selected from N, O, and S; wherein each C₁-C₈ alkyl of R⁴ is independently, optionally substituted with one, two or three R^{f} substituents;
each R^{d} is independently carbonyl or C₁-C₆ alkyl;
each R^{e} is independently halo, cyano, or C₁-C₆ alkyl; and
each R^{f} is independently halo, cyano, or phenyl;
wherein when the prodrug of the compound of Formula A, the prodrug of the deuterated compound of Formula A, or the pharmaceutically acceptable salt thereof is administered, the prodrug of the compound of Formula A, the prodrug of the deuterated compound of Formula A, or the pharmaceutically acceptable salt thereof is converted substantially to the compound of Formula A or the deuterated compound of Formula A; and
wherein the administration results in a mean Cₘₐₓ of less than 7,000 ng/mL of the compound of Formula A, or the deuterated compound of Formula A.

In some embodiments, the administration of the compound of Formula A, the deuterated compound of Formula A, the prodrug of Formula A, the prodrug of the deuterated compound of Formula A, or a pharmaceutically acceptable salt thereof, results in a mean Cₘₐₓ of 1,000 ng/mL to 6,500 ng/mL, 1,000 ng/mL to 6,000 ng/mL, 1,000 ng/mL to 5,500 ng/mL, 1,000 ng/mL to 5,000 ng/mL, 1,000 ng/mL to 4500 ng/mL, 2,000 ng/mL to 6,500 ng/mL, 2,000 ng/mL to 6,000 ng/mL, 2,000 ng/mL to 5,500 ng/mL, 2,000 ng/mL to 5,000 ng/mL, 2,000 ng/mL to 4500 ng/mL, 2,500 ng/mL to 6,500 ng/mL, 2,500 ng/mL to 6,000 ng/mL, 2,500 ng/mL to 5,500 ng/mL, 2,500 ng/mL to 5,000 ng/mL, or 2,500 ng/mL to 4500 ng/mL of the compound of Formula A, or the deuterated compound of Formula A. In some embodiments, the administration of the compound of Formula A, the deuterated compound of Formula A, the prodrug of Formula A, the prodrug of the deuterated compound of Formula A, or a pharmaceutically acceptable salt thereof, results in a mean Cₘₐₓ of 2,000 ng/mL to 6,200 ng/mL of the compound of Formula A, or the deuterated compound of Formula A. In some embodiments, the administration of the compound of Formula A, the deuterated compound of Formula A, the prodrug of Formula A, the prodrug of the deuterated compound of Formula A, or a pharmaceutically acceptable salt thereof, results in a mean Cₘₐₓ of 2,000 ng/mL to 4,900 ng/mL of the compound of Formula A, or the deuterated compound of Formula A.

In some embodiments, the administration of the compound of Formula A, the deuterated compound of Formula A, the prodrug of Formula A, the prodrug of the deuterated compound of Formula A, or a pharmaceutically acceptable salt thereof, results in a mean AUC₀₋₂₄ of less than 44,000 ng/mL*h the compound of Formula A, or the deuterated compound of Formula A. In some embodiments, the administration of the compound of Formula A, the deuterated compound of Formula A, the prodrug of Formula A, the prodrug of the deuterated compound of Formula A, or a pharmaceutically acceptable salt thereof, results in a mean AUC₀₋₂₄ of 12,000 ng/mL*h to 44,000 ng/mL*h, 12,000 ng/mL*h to 40,000 ng/mL*h, 12,000 ng/mL*h to 36,000 ng/mL*h, 12,000 ng/mL*h to 32,000 ng/mL*h, 16,000 ng/mL*h to 44,000 ng/mL*h, 16,000 ng/mL*h to 40,000 ng/mL*h, 16,000 ng/mL*h to 36,000 ng/mL*h, 16,000 ng/mL*h to 32,000 ng/mL*h, 20,000 ng/mL*h to 44,000 ng/mL*h, 20,000 ng/mL*h to 40,000 ng/mL*h, 20,000 ng/mL*h to 36,000 ng/mL*h, or 20,000 ng/mL*h to 32,000 ng/mL*h of the compound of Formula A, or the deuterated compound of Formula A. In some embodiments, the administration of the compound of Formula A, the deuterated compound of Formula A, the prodrug of Formula A, the prodrug of the deuterated compound of Formula A, or a pharmaceutically acceptable salt thereof, results in a mean AUC₀₋₂₄ of 18,200 ng/mL*h to 36,400 ng/mL*h of the compound of Formula A, or the deuterated compound of Formula A. In some embodiments, the administration of the compound of Formula A, the deuterated compound of Formula A, the prodrug of Formula A, the prodrug of the deuterated compound of Formula A, or a pharmaceutically acceptable salt thereof, results in a mean AUC₀₋₂₄ of 18,200 ng/mL*h to 32,000 ng/mL*h of the compound of Formula A, or the deuterated compound of Formula A.

In some embodiments, upon administration to the patient, greater than 60% of the prodrug of the compound of Formula A, the prodrug of the deuterated compound of Formula A, or the pharmaceutically acceptable salt thereof is converted to the compound of Formula A or the deuterated compound of Formula A. In some embodiments, upon administration to the patient, greater than 70% of the prodrug of the compound of Formula A, the prodrug of the deuterated compound of Formula A, or the pharmaceutically acceptable salt thereof is converted to the compound of Formula A or the deuterated compound of Formula A. In some embodiments, upon administration to the patient, greater than 80% of the prodrug of the compound of Formula A, the prodrug of the deuterated compound of Formula A, or the pharmaceutically acceptable salt thereof is converted to the compound of Formula A or the deuterated compound of Formula A. In some embodiments, upon administration to the patient, greater than 90% of the prodrug of the compound of Formula A, the prodrug of the deuterated compound of Formula A, or the pharmaceutically acceptable salt thereof is converted to the compound of Formula A or the deuterated compound of Formula A. In some embodiments, upon administration to the patient, greater than 95% of the prodrug of the compound of Formula A, the prodrug of the deuterated compound of Formula A, or the pharmaceutically acceptable salt thereof is converted to the compound of Formula A or the deuterated compound of Formula A. In some embodiments, upon administration to the patient, greater than 95% of the prodrug of the compound of Formula A, the prodrug of the deuterated compound of Formula A, or the pharmaceutically acceptable salt thereof is converted to the compound of Formula A or the deuterated compound of Formula A.

In some embodiments, the carbon bonded to the 5 position on the tetrahydrofuranyl ring of the deuterated compound of Formula A is substituted with one or two deuterium atoms. In some embodiments, the deuterated compound of Formula A is

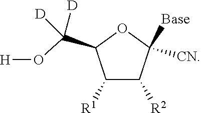

In some embodiments, the deuterated compound of Formula A is

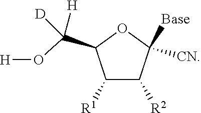

In some embodiments, a carbon of the Base of the deuterated compound of Formula A is substituted with one or more deuterium atoms. In some embodiments, Base is

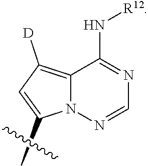

In some embodiments, Base is

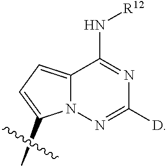

In some embodiments, Base is

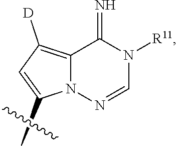

In some embodiments, Base is

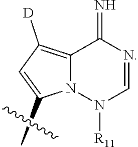

In some embodiments, a carbon of R¹² of the Base of the deuterated compound of Formula A is substituted with one or more deuterium atoms, e.g., one or two deuterium atoms. In some embodiments, a carbon on R¹¹ of the Base of the deuterated compound of Formula A is substituted with one or more deuterium atoms, e.g., one or two deuterium atoms.

In some embodiments, a carbon of R¹ of the deuterated compound of Formula A is substituted with one or more deuterium atoms, e.g., one or two deuterium atoms. In some embodiments, a carbon of R² of the deuterated compound of Formula A is substituted with one or more deuterium atoms, e.g., one or two deuterium atoms.

In some embodiments, the compound of Formula A is

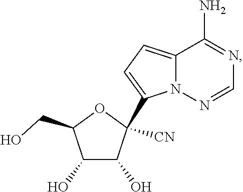 [SMILES: N#C[C@@]1(c2ccc3c(N)ncnn23)O[C@H](CO)[C@@H](O)[C@H]1O]

or a pharmaceutically acceptable salt thereof.

In some embodiments, the deuterated compound of Formula A is

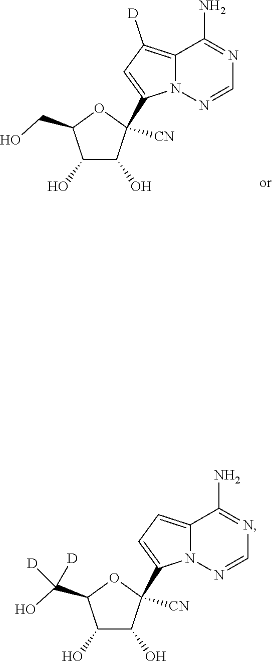 [SMILES: [2H]c1cc([C@]2(C#N)O[C@H](CO)[C@@H](O)[C@H]2O)n2ncnc(N)c12 ; [2H]C([2H])(O)[C@H]1O[C@@](C#N)(c2ccc3c(N)ncnn23)[C@H](O)[C@@H]1O]

or a pharmaceutically acceptable salt thereof.

In some embodiments, the method comprises administering to the patient a prodrug of Formula A having Formula I:

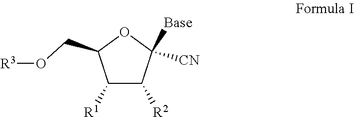

or a deuterated compound of Formula I, or a pharmaceutically acceptable salt thereof, wherein:
R³ is —C(═O)OR⁷, or —C(═O)R⁷; and
R⁷ is H, C₁-C₈ alkyl, C₂-C₈ alkenyl, C₂-C₈ alkynyl, C₃-C₈ carbocyclyl, C₆-C₁₀ aryl, 4 to 6 membered heterocyclyl containing 1, 2, or 3 heteroatoms selected from N, O, and S, or 5 to 6 membered heteroaryl containing 1, 2, or 3 heteroatoms selected from N, O, and S; wherein C₁-C₈ alkyl, C₂-C₈ alkenyl, C₂-C₈ alkynyl, C₃-C₈ carbocyclyl, C₆-C₁₀ aryl, 4 to 6 membered heterocyclyl, and 5 to 6 membered heteroaryl of R⁷ are optionally substituted with one, two, or three R^{a} substituents.

In some embodiments, a carbon of R³ of the deuterated compound of Formula I is substituted with one or more deuterium atoms, e.g., one or two deuterium atoms. In some embodiments, a carbon of R⁷ of the deuterated compound of Formula I is substituted with one or more deuterium atoms, e.g., one or two deuterium atoms.

In some embodiments, R⁷ is —OH. In some embodiments, R¹ is —OC(═O)R⁴. In some embodiments, R¹ is —OC(═O)OR⁴.

In some embodiments, R² is —OH. In some embodiments, R² is —OC(═O)R⁵. In some embodiments, R² is —OC(═O)OR⁵.

In some embodiments, R¹ and R² are both —OH. In some embodiments, R¹ is —OC(═O)R⁴ and R² is —OC(═O)R⁵. In some embodiments, R¹ is OH, and R² is —OC(═O)R⁵ or —OC(═O)OR⁵—. In some embodiments, R¹ is —OC(═O)R⁴ or —OC(═O)OR⁴, and R² is OH. In some embodiments, R¹ and R² are taken together to form —OC(═O)O— In some embodiments, R¹ and R² are taken together to form —OP(═O)(OH)O—. In some embodiments, R¹ and R² are taken together to form —OCHR⁶O—.

In some embodiments,
R¹ is OH, OC(O)CH(CH₃)₂, or OC(O)CH₃,
R² is OH, OC(O)CH(CH₃)₂, or OC(O)CH₃, or
R¹ and R² are taken together to form —OC(═O)O—.

In some embodiments, R⁴ is C₁-C₈ alkyl, C₂-C₈ alkenyl, or C₂-C₈ alkynyl. In some embodiments, R⁴ is C₁-C₈ alkyl, C₂-C₈ alkenyl, or C₂-C₈ alkynyl, each optionally substituted with one, two, or three R^{a} substituents independently selected from halo, cyano, —N₃, —OR⁸, —NR⁹R¹⁰, —OP(═O)(OH)₂, C₃-C₈ carbocyclyl and phenyl.

In some embodiments, R⁴ is C₁-C₈ alkyl. In some embodiments, R⁴ is C₁-C₈ alkyl optionally substituted with one, two, or three R substituents independently selected from halo, cyano, —N₃, —OR⁸, —NR⁹R¹⁰, —OP(═O)(OH)₂, C₃-C₈ carbocyclyl and phenyl. In some embodiments, R⁴ is unsubstituted C₁-C₈ alkyl. In some embodiments, R⁴ is unsubstituted C₁-C₆ alkyl. In some embodiments, R⁴ is unsubstituted C₁-C₃ alkyl. In some embodiments, R⁴ is —CH₃, —CH₂CH₃, —(CH₂)₂CH₃, —CH(CH₃)₂, —(CH₂)₃CH₃, or —C(CH₃)₃. In some embodiments, R⁴ is —CH₃ or —CH(CH₃)₂.

In some embodiments, R⁵ is C₁-C₈ alkyl, C₂-C₈ alkenyl, or C₂-C₈ alkynyl. In some embodiments, R⁴ is C₁-C₈ alkyl, C₂-C₈ alkenyl, or C₂-C₈ alkynyl, each optionally substituted with one, two, or three R^{a} substituents independently selected from halo, cyano, —N₃, —OR⁸, —NR⁹R¹⁰, —OP(═O)(OH)₂, C₃-C₈ carbocyclyl and phenyl.

In some embodiments, R⁵ is C₁-C₈ alkyl. In some embodiments, R⁵ is C₁-C₈ alkyl optionally substituted with one, two, or three R^{a} substituents independently selected from halo, cyano, —N₃, —OR⁸, —NR⁹R¹⁰, —OP(═O)(OH)₂, C₃-C₈ carbocyclyl and phenyl. In some embodiments, R⁵ is unsubstituted C₁-C₈ alkyl. In some embodiments, R⁵ is unsubstituted C₁-C₆ alkyl. In some embodiments, R⁵ is unsubstituted C₁-C₃ alkyl. In some embodiments, R⁵ is —CH₃, —CH₂CH₃, —(CH₂)₂CH₃, —CH(CH₃)₂, —(CH₂)₃CH₃, or —C(CH₃)₃. In some embodiments, R⁵ is —CH₃ or —CH(CH₃)₂.

In some embodiments, R⁴ and R⁵ are the same. In some embodiments, R⁴ and R⁵ are different. In some embodiments, R⁴ is C₁-C₈ alkyl and R⁵ is C₁-C₈ alkyl. In some embodiments, R⁴ is unsubstituted C₁-C₈ alkyl and R⁵ is unsubstituted C₁-C₈ alkyl. In some embodiments, R⁴ is —CH₃ or —CH(CH₃)₂ and R⁵ is —CH or —CH(CH₃)₂. In some embodiments, R⁴ is —CH₃; and R⁵ is —CH₃. In some embodiments, R⁴ is —CH(CH₃)₂ and R⁵ is —CH(CH₃)₂.

In some embodiments, R⁶ is H. In some embodiments, R⁶ is C₁-C₆ alkyl. In some embodiments, R⁶ is —CH₃, —CH₂CH₃, —(CH₂)₂CH₃, —CH(CH₃)₂, —(CH₂)₃CH₃, or —C(CH₃)₃. In some embodiments, R⁶ is C₁-C₆ alkoxy.

In some embodiments, R⁶ is 5 to 6 membered heteroaryl containing 1, 2, or 3 heteroatoms selected from N, O, and S. In some embodiments, R⁶ is 5 to 6 membered heteroaryl containing 1, 2, or 3 heteroatoms selected from N, O, and S substituted with one, two, or three R^{b} substitutents. In some embodiments, R⁶ is 5 to 6 membered heteroaryl containing 1, 2, or 3 heteroatoms selected from N, O, and S. In some embodiments, R⁶ is unsubstituted 5 to 6 membered heteroaryl containing 1, 2, or 3 heteroatoms selected from N, O, and S.

In some embodiments, R⁶ is C₆-C₁₀ aryl. In some embodiments, R⁶ is C₆-C₁₀ aryl substituted with one, two, or three R^{b} substituents. In some embodiments, R⁶ is unsubstituted C₆-C₁₀ aryl. In some embodiments, R⁶ is phenyl. In some embodiments, R⁶ is unsubstituted phenyl.

In some embodiments, R³ is —C(═O)OR⁷. In some embodiments, R³ is —C(═O)R⁷.

In some embodiments, R⁷ is C₁-C₈ alkyl, C₃-C₈ carbocyclyl, C₆-C₁₀ aryl, 4 to 6 membered heterocyclyl containing 1, 2, or 3 heteroatoms selected from N, O, and S, or 5 to 6 membered heteroaryl containing 1, 2, or 3 heteroatoms selected from N, O, and S. In some embodiments, R⁷ is C₁-C₈ alkyl, C₃-C₈ carbocyclyl, C₆-C₁₀ aryl, 4 to 6 membered heterocyclyl containing 1, 2, or 3 heteroatoms selected from N, O, and S, or 5 to 6 membered heteroaryl containing 1, 2, or 3 heteroatoms selected from N, O, and S, each optionally substituted with one, two, or three R^{a} substituents independently selected from —OR⁸, —OP(═O)(OH)₂, C₃-C₈ carbocyclyl and phenyl.

In some embodiments, R⁷ is C₁-C₈ alkyl, C₂-C₈ alkenyl, or C₂-C₈ alkynyl. In some embodiments, R⁷ is C₁-C₈ alkyl, C₂-C₈ alkenyl, or C₂-C₈ alkynyl, each optionally substituted with one, two, or three R^{a} substituents independently selected from halo, cyano, —N₃, —OR⁸, —NR⁹R¹⁰, —OP(═O)(OH)₂, C₃-C₈ carbocyclyl and phenyl. In some embodiments, R⁷ is C₁-C₈ alkyl, C₂-C₈ alkenyl, or C₂-C₈ alkynyl, each optionally substituted with one, two, or three R^{a} substituents independently selected from halo, cyano, —N₃, —OR⁸, —NR⁹R¹⁰, and phenyl.

In some embodiments, R⁷ is C₁-C₈ alkyl. In some embodiments, R⁷ is C₁-C₈ alkyl optionally substituted with one, two, or three R substituents independently selected from halo, cyano, —N₃, —OR⁸, —NR⁹R¹⁰, —OP(═O)(OH)₂, C₃-C₈ carbocyclyl and phenyl. In some embodiments, R⁷ is C₁-C₈ alkyl optionally substituted with one R substituent selected from —OR⁸, —OP(═O)(OH)₂, C₃-C₈ carbocyclyl and unsubstituted phenyl. In some embodiments, R⁷ is C₁-C₈ alkyl optionally substituted with one, two, or three R^{a} substituents independently selected from halo, cyano, —N₃, —OR⁸, —NR⁹R¹⁰, and phenyl. In some embodiments, R⁷ is C₁-C₈ alkyl optionally substituted with one, two, or three R^{a} substituents independently selected from halo, cyano, —N₃, —OR⁸, —NR⁹R¹⁰, and unsubstituted phenyl. In some embodiments, R⁷ is C₁-C₈ alkyl substituted with —OR⁸. In some embodiments, R⁷ is C₁-C₈ alkyl substituted with phenyl. In some embodiments, R⁷ is C₁-C₈ alkyl substituted with C₃-C₈ carbocyclyl. In some embodiments, R⁷ is C₁-C₈ alkyl substituted with —OP(═O)(OH)₂.

In some embodiments, R⁷ is C₁-C₆ alkyl. In some embodiments, R⁷ is C₁-C₆ alkyl optionally substituted with one, two, or three R^{a} substituents independently selected from halo, cyano, —N₃, —OR⁸, —NR⁹R¹⁰, —OP(═O)(OH)₂, C₃-C₈ carbocyclyl and phenyl. In some embodiments, R⁷ is C₁-C₆ alkyl optionally substituted with one R substituent selected from —OR⁸, —OP(═O)(OH)₂, C₃-C₈ carbocyclyl and unsubstituted phenyl.

In some embodiments, R⁷ is C₁-C₄ alkyl substituted with phenyl. In some embodiments, R⁷ is C₁-C₄ alkyl substituted with phenyl. R⁷ is C₁-C₄ alkyl substituted with —OP(═O)(OH)₂.

In some embodiments, R⁷ is C₂-C₄ alkyl. In some embodiments, R⁷ is C₂-C₄ alkyl substituted with —OR⁸. In some embodiments, R⁷ is C₂-C₄ alkyl substituted with —OCH₃. In some embodiments, R⁷ is C₂-C₄ alkyl substituted with C₄-C₇ carbocyclyl. In some embodiments, R⁷ is C₂-C₄ alkyl substituted with

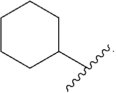 [SMILES: *C1CCCCC1]

In some embodiments, R⁷ is —CH₃, —CH₂CH₃,

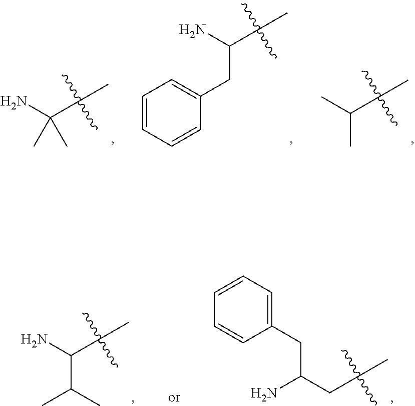

In some embodiments, R⁷—CH₃, —CH₂CH₃,

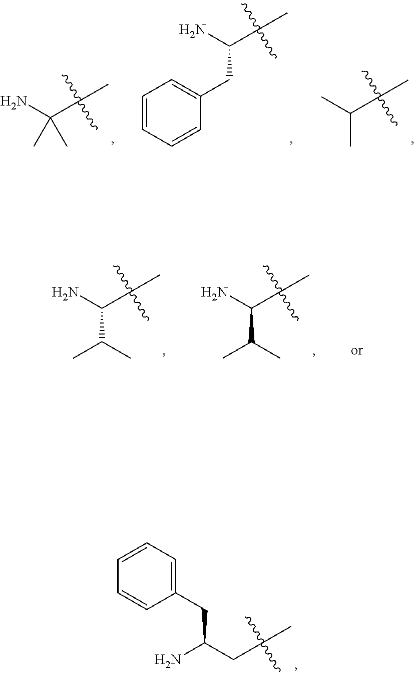

In some embodiments, R⁷ is —CH₃, —CH₂CH₃,

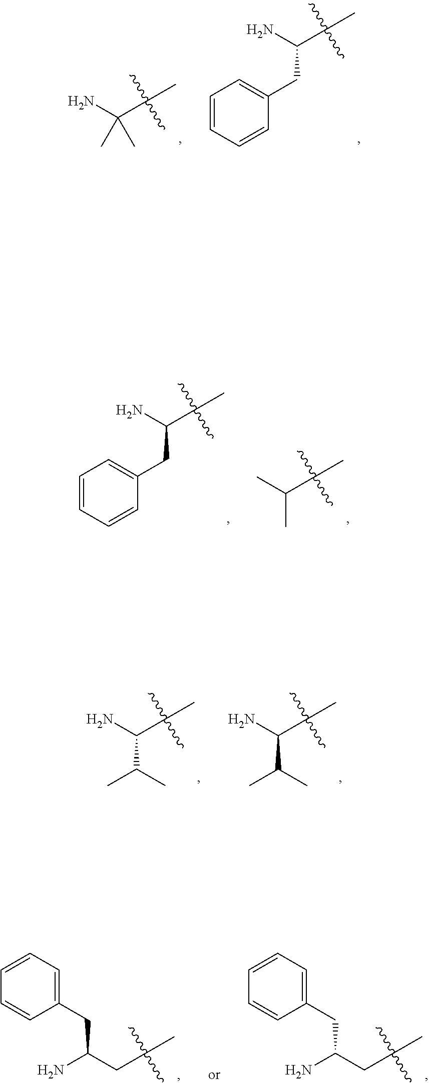

In some embodiments R⁷ is

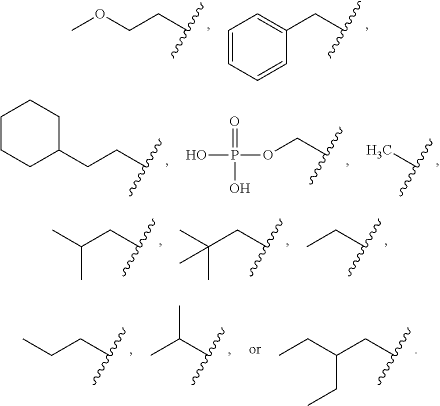

In some embodiments, R⁷ is

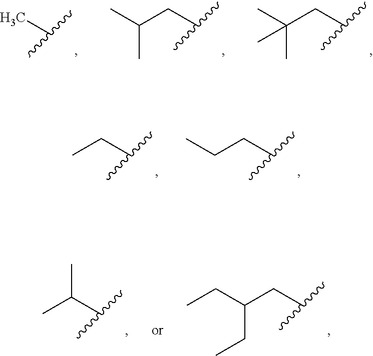

In some embodiments, R⁷ is

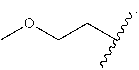 [SMILES: *CCOC]

In some embodiments, R⁷ is

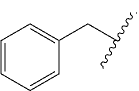 [SMILES: *Cc1ccccc1]

In some embodiments, R⁷ is

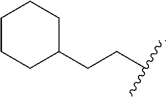 [SMILES: *CCC1CCCCC1]

In some embodiments, R⁷ is

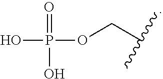 [SMILES: *COP(=O)(O)O]

In some embodiments, R⁷ is C₃-C₈ carbocyclyl. In some embodiments, R⁷ is C₃-C₈ carbocyclyl optionally substituted with one, two, or three R^{a} substituents independently selected from halogen, cyano, —N₃, —OR⁸, —NR⁹R¹⁰, —OP(═O)(OH)₂, C₃-C₈ carbocyclyl and phenyl. In some embodiments, R⁷ is C₃-C₈ carbocyclyl optionally substituted with one, two, or three R^{a} substituents independently selected from halogen, cyano, —N₃, —OR⁸, —NR⁹R¹⁰, —OP(═O)(OH)₂, C₃-C₈ carbocyclyl and unsubstituted phenyl. In some embodiments, R⁷ is C₃-C₈ carbocyclyl optionally substituted with one, two, or three substituents independently selected from —OR⁸, —NR⁹R¹⁰, C₃-C₈ carbocyclyl and unsubstituted phenyl.

In some embodiments, R⁷ is

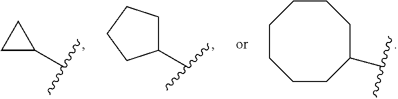

In some embodiments, R⁷ is C₆-C₁₀ aryl. In some embodiments, R⁷ is C₆-C₁₀ aryl optionally substituted with one, two, or three R^{a} substituents independently selected from halogen, cyano, —N₃, —OR⁸, —NR⁹R¹⁰, —OP(═O)(OH)₂, C₃-C₈ carbocyclyl and phenyl. In some embodiments, R⁷ is C₆-C₁₀ aryl optionally substituted with one, two, or three R^{a} substituents independently selected from halogen, cyano, —N₃, —OR⁸, —NR⁹R¹⁰, —OP(═O)(OH)₂, C₃-C₈ carbocyclyl and unsubstituted phenyl.

In some embodiments, R⁷ is phenyl or naphthyl. In some embodiments, R⁷ is phenyl or naphthyl, each optionally substituted with one, two, or three R^{a} substituents independently selected from halogen, cyano, —N₃, —OR⁸, —NR⁹R¹⁰, —OP(═O)(OH)₂, C₃-C₈ carbocyclyl and unsubstituted phenyl. In some embodiments, R⁷ is phenyl. In some embodiments, R⁷ is phenyl optionally substituted with one, two, or three R^{a} substituents independently selected from halogen, cyano, —N₃, —OR⁸, —NR⁹R¹⁰, —OP(═O)(OH)₂, C₃-C₈ carbocyclyl and unsubstituted phenyl.

In some embodiments, R⁷ is

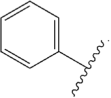 [SMILES: *c1ccccc1]

In some embodiments, R⁷ is 4 to 6 membered heterocyclyl containing 1, 2, or 3 heteroatoms selected from N, O, and S. In some embodiments, R⁷ is 4 to 6 membered heterocyclyl containing 1, 2, or 3 heteroatoms selected from N, O, and S optionally substituted with one, two, or three R^{a} substituents independently selected from halogen, cyano, —N₃, —OR⁸, —NR⁹R¹⁰, —OP(═O)(OH)₂, C₃-C₈ carbocyclyl and phenyl. In some embodiments, R⁷ is 4 to 6 membered heterocyclyl containing 1, 2, or 3 heteroatoms selected from N, O, and S optionally substituted with one, two, or three R^{a} substituents independently selected from halogen, cyano, —N₃, —OR⁸, —NR⁹R¹⁰, —OP(═O)(OH)₂, C₃-C₈ carbocyclyl and unsubstituted phenyl.

In some embodiments, R⁷ is 6 membered heterocyclyl containing 1, 2, or 3 heteroatoms selected from N, O, and S. In some embodiments, R⁷ is 6 membered heterocyclyl containing 1, 2, or 3 heteroatoms selected from N, O, and S optionally substituted with one, two, or three R^{a} substituents independently selected from halogen, cyano, —N₃, —OR⁸, —NR⁹R¹⁰, —OP(═O)(OH)₂, C₃-C₈ carbocyclyl and unsubstituted phenyl.

In some embodiments, R⁷ is 5 membered heterocyclyl containing 1, 2, or 3 heteroatoms selected from N, O, and S. In some embodiments, R⁷ is 5 membered heterocyclyl containing 1, 2, or 3 heteroatoms selected from N, O, and S optionally substituted with one, two, or three R^{a} substituents independently selected from halogen, cyano, —N₃, —OR⁸, —NR⁹R¹⁰, —OP(═O)(OH)₂, C₃-C₈ carbocyclyl and unsubstituted phenyl. In some embodiments, R⁷ is unsubstituted 5 membered heterocyclyl containing 1, 2, or 3 heteroatoms selected from N, O, and S.

In some embodiments, R⁷ is

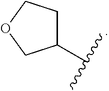 [SMILES: *C1CCOC1]

In some embodiments, R⁷ is 4 membered heterocyclyl containing 1, 2, or 3 heteroatoms selected from N, O, and S. In some embodiments, R⁷ is unsubstituted 4 membered heterocyclyl containing 1, 2, or 3 heteroatoms selected from N, O, and S.

In some embodiments, R⁷ is

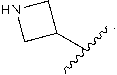 [SMILES: *C1CNC1]

In some embodiments, R⁷ is 5 to 6 membered heteroaryl containing 1, 2, or 3 heteroatoms selected from N, O, and S. In some embodiments, R⁷ is 5 to 6 membered heteroaryl containing 1, 2, or 3 heteroatoms selected from N, O, and S optionally substituted with one, two, or three R^{a} substituents independently selected from halogen, cyano, —N₃, —OR⁸, —NR⁹R¹⁰, —OP(═O)(OH)₂, C₃-C₈ carbocyclyl and phenyl. In some embodiments, R⁷ is 5 to 6 membered heteroaryl containing 1, 2, or 3 heteroatoms selected from N, O, and S optionally substituted with one, two, or three R^{a} substituents independently selected from halogen, cyano, —N₃, —OR, —NR⁹R¹⁰, —OP(═O)(OH)₂, C₃-C₈ carbocyclyl and unsubstituted phenyl.

In some embodiments, R⁷ is 5 membered heteroaryl containing 1, 2, or 3 heteroatoms selected from N, O, and S. In some embodiments, R⁷ is 5 membered heteroaryl containing 1, 2, or 3 heteroatoms selected from N, O, and S optionally substituted with one, two, or three R^{a} substituents independently selected from halogen, cyano, —N₃, —OR⁸, —NR⁹R¹⁰, —OP(═O)(OH)₂, C₃-C₈ carbocyclyl and unsubstituted phenyl. In some embodiments, R⁷ is unsubstituted 5 membered heteroaryl containing 1, 2, or 3 heteroatoms selected from N, O, and S.

In some embodiments, R⁷ is 6 membered heteroaryl containing 1, 2, or 3 heteroatoms selected from N, O, and S. In some embodiments, R⁷ is 6 membered heteroaryl containing 1, 2, or 3 heteroatoms selected from N, O, and S optionally substituted with one, two, or three R^{a} substituents independently selected from halogen, cyano, —N₃, —OR⁸, —NR⁹R¹⁰, —OP(═O)(OH)₂, C₃-C₈ carbocyclyl and unsubstituted phenyl. In some embodiments, R⁷ is 6 membered heteroaryl containing 1, 2, or 3 heteroatoms selected from N, O, and S optionally substituted with one, two, or three R^{a} substituents independently selected from halogen, cyano, and —NR⁹R¹⁰. In some embodiments, R⁷ is unsubstituted 6 membered heteroaryl containing 1, 2, or 3 heteroatoms selected from N, O, and S.

In some embodiments, R⁷ is

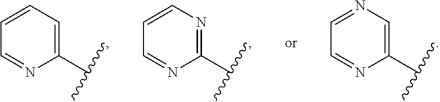 [SMILES: *c1ccccn1 ; *c1ncccn1 ; *c1cnccn1]

In some embodiments, R⁷ is

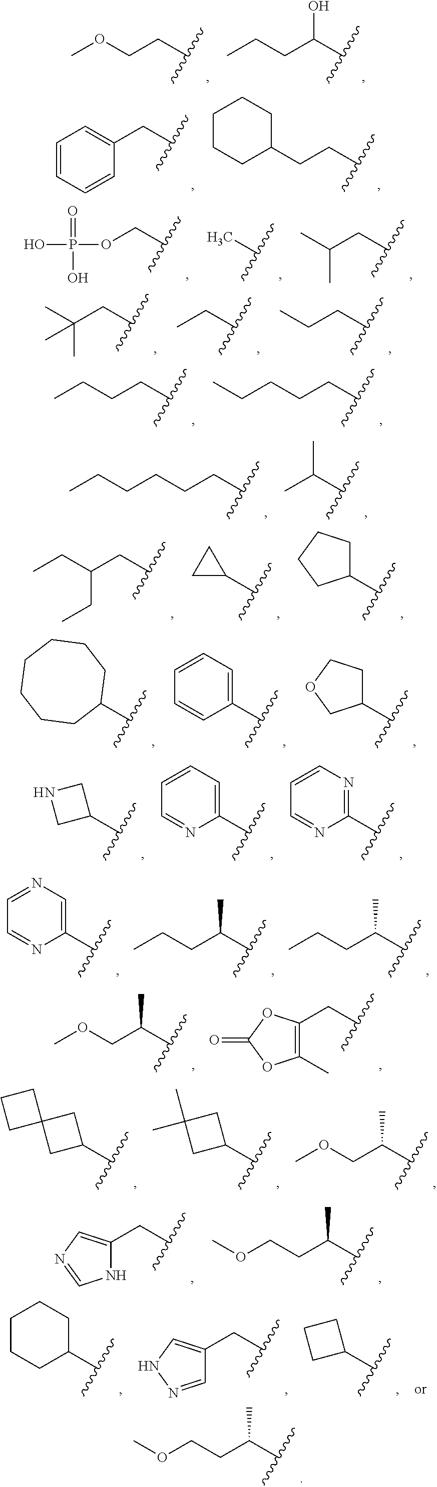

In some embodiments, R⁷ is

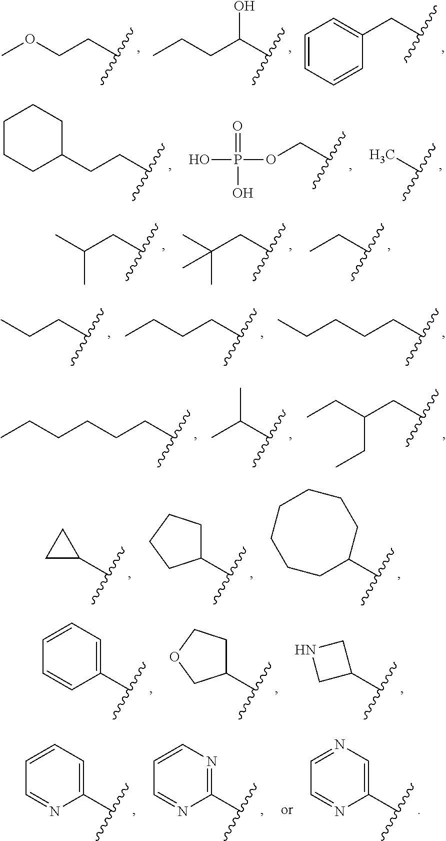

In some embodiments, R⁷ is

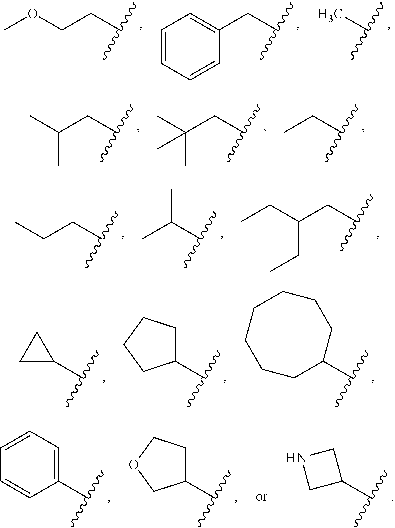

In some embodiments, R⁸ is H. In some embodiments, R⁸ is C₁-C₆ alkyl. In some embodiments, R⁸ is —CH₃. In some embodiments, R⁸ is C₁-C₆ haloalkyl. In some embodiments, R⁸ is C₃-C₆ cycloalkyl.

In some embodiments, R⁹ is H. In some embodiments, R⁹ is C₁-C₆ alkyl. In some embodiments, R⁹ is —CH₃. In some embodiments, R⁹ is C₁-C₆ haloalkyl. In some embodiments, R⁹ is C₃-C₆ cycloalkyl.

In some embodiments, R¹⁰ is H. In some embodiments, R¹⁰ is C₁-C₆ alkyl. In some embodiments, R¹⁰ is —CH₃. In some embodiments, R¹⁰ is C₁-C₆ haloalkyl. In some embodiments, R¹⁰ is C₃-C₆ cycloalkyl.

In some embodiments, Base is

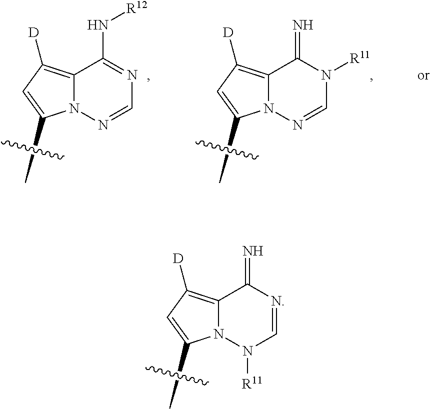

In some embodiments, Base is

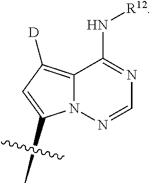

In some embodiments, Base is

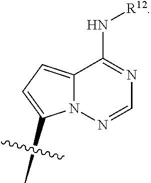

In some embodiments, Base is

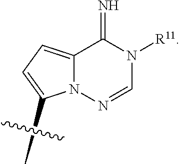

In some embodiments, Base is

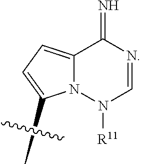

In some embodiments, Base is

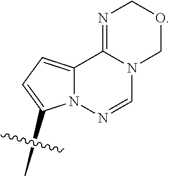

In some embodiments, R¹¹ is C₁-C₃ alkyl substituted with —OP(═O)(OH)(OR¹⁴). In some embodiments, R¹¹ is —(CH₂)OP(═O)(OH)(OR⁴).

In some embodiments, R¹⁴ is H.

In some embodiments, R¹⁴ is H or C₁-C₈ alkyl; wherein C₁-C₈ alkyl of R¹⁴ is optionally substituted with one, two or three substituents independently selected from halogen, cyano, and phenyl.

In some embodiments, R¹⁴ is C₁-C₈ alkyl optionally substituted with one, two or three substituents independently selected from halogen, cyano, and phenyl. In some embodiments, R¹⁴ is C₁-C₃ alkyl optionally substituted with one, two or three substituents independently selected from halogen, cyano, and phenyl. In some embodiments, R¹⁴ is C₁-C₃ alkyl substituted with one phenyl. In some embodiments, R¹⁴ is

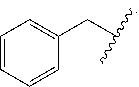 [SMILES: *Cc1ccccc1]

In some embodiments, R¹¹ is —(CH₂)OP(═O)(OH)₂. In some embodiments, R¹¹ is

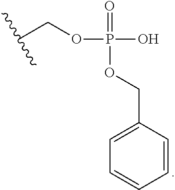 [SMILES: *COP(=O)(O)OCc1ccccc1]

In some embodiments, R¹² is H. In some embodiments, R¹² is C₁-C₆ alkyl. In some embodiments, R¹² is —C(═O)R¹³. In some embodiments, R¹² is —C(═O)(CH₂)₂CH₃. In some embodiments, R¹² is —C(═O)OR¹³. In some embodiments, R¹² is —C(═O)OCH₂CH(CH₃)₂. In some embodiments, R¹² is —C(═O)OCH₂CH(CH₃)₂ or —C(═O)(CH₂)₂CH₃. In some embodiments, R¹² is

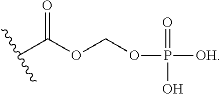 [SMILES: *C(=O)OCOP(=O)(O)O]

In some embodiments, R¹³ is H. In some embodiments, R¹³ is C₁-C₁₀ alkyl. In some embodiments, R¹³ is C₁-C₈ alkyl. In some embodiments, R¹³ is C₁-C₈ alkyl optionally substituted with one, two, or three R^{c} substituents independently selected from halogen, cyano, —OP(═O)(OH)(OR¹⁴) and phenyl. In some embodiments, R¹³ is C₁-C₈ alkyl optionally substituted with one, two, or three R^{c} substituents independently selected from halogen, cyano, and phenyl. In some embodiments, R¹³ is C₁-C₈ alkyl optionally substituted with one, two, or three R^{c} substituents independently selected from halogen, cyano, and unsubstituted phenyl. In some embodiments, R¹³ is C₁-C₈ alkyl. In some embodiments, R¹³ is —CH₃, —CH₂CH₃, —(CH₂)₂CH₃, —CH(CH₃)₂, —(CH₂)₃CH₃, or —C(CH₃)₃. In some embodiments, R¹³ is —CH₂CH(CH₃)₂ or —(CH₂)₂CH₃. In some embodiments, R¹³ is —(CH₂)OP(═O)(OH)₂. In some embodiments, R¹³ is C₆-C₁₀ aryl. In some embodiments, R¹³ is phenyl. In some embodiments, R¹³ is —O—C₆-C₁₀ aryl. In some embodiments, R¹³ is —O-phenyl. In some embodiments, R¹³ is —O—C₁-C₁₀ alkyl. In some embodiments, R¹³ is —O—CH₃, —O—CH₂CH₃, —O—(CH₂)₂CH₃, —O—(CH₂)₄CH₃, or —O—(CH₂)₆CH₃.

In some embodiments, R¹³ is

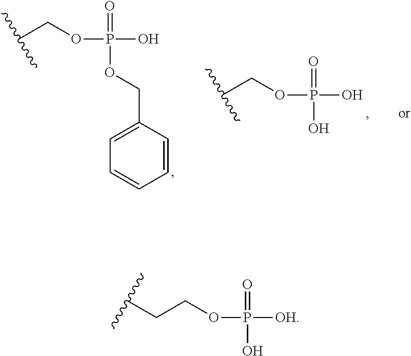 [SMILES: *COP(=O)(O)OCc1ccccc1 ; *COP(=O)(O)O ; *CCOP(=O)(O)O]

In some embodiments, Base is

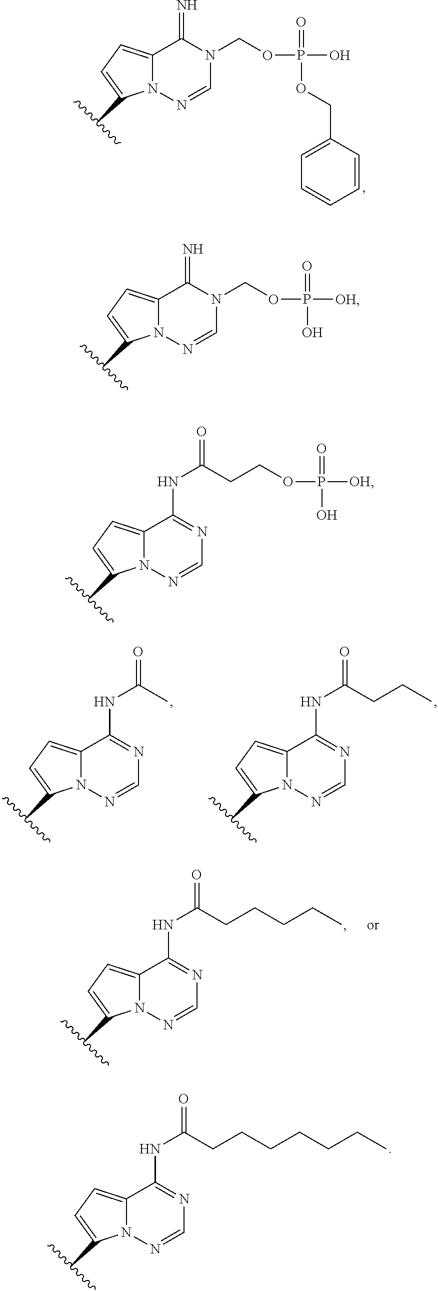

In some embodiments, the method comprises administering to the patient a compound of Formula A or a prodrug thereof, or a pharmaceutically acceptable salt thereof, wherein
R¹ is —OH, —OC(═O)R⁴, or —OC(═O)OR⁴;
R² is —OH, —OC(═O)R⁵, or —OC(═O)OR⁵; or
R¹ and R² are taken together to form —OC(═O)O—; and
R⁴, and R⁵ are each independently unsubstituted C₁-C₈ alkyl, unsubstituted C₃-C₈ carbocyclyl, unsubstituted C₆-C₁₀ aryl, unsubstituted 4 to 6 membered heterocyclyl containing 1, 2, or 3 heteroatoms selected from N, O, and S, or unsubstituted 5 to 6 membered heteroaryl containing 1, 2, or 3 heteroatoms selected from N, O, and S.

In some embodiments, Base is

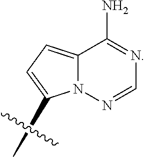

In some embodiments, R⁴ and R⁵ are each independently unsubstituted C₁-C₈ alkyl. In some embodiments, R⁴ and R⁵ are each independently unsubstituted C₁-C₆ alkyl. In some embodiments, R¹ is —OH or —OC(═O)R⁴ and R² is —OH or —OC(═O)R⁵. In some embodiments, R¹ and R² are each independently —OH, OC(═O)CH(CH₃)₂, or OC(═O)CH₃. In some embodiments, R¹ and R² are taken together to form —OC(═O)O—.

In some embodiments, the method comprises administering to the patient a deuterated compound of Formula A which has Formula A1

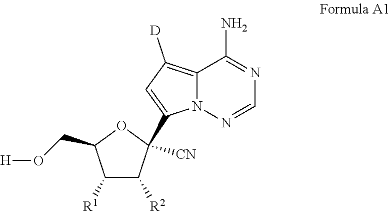

or a prodrug thereof, or a pharmaceutically acceptable salt thereof, wherein
R¹ is —OH, —OC(═O)R⁴, or —OC(═O)OR⁴;
R² is —OH, —OC(═O)R⁵, or —OC(═O)OR⁵; or
R¹ and R² are taken together to form —OC(═O)O—; and
R⁴, and R⁵ are each independently unsubstituted C₁-C₈ alkyl, unsubstituted C₃-C₈ carbocyclyl, unsubstituted C₆-C₁₀ aryl, unsubstituted 4 to 6 membered heterocyclyl containing 1, 2, or 3 heteroatoms selected from N, O, and S, or unsubstituted 5 to 6 membered heteroaryl containing 1, 2, or 3 heteroatoms selected from N, O, and S.

In some embodiments, R⁴ and R⁵ are each independently unsubstituted C₁-C₈ alkyl. In some embodiments, R⁴ and R⁵ are each independently unsubstituted C₁-C₆ alkyl. In some embodiments, R¹ is —OH or —OC(═O)R⁴ and R² is —OH or —OC(═O)R⁵. In some embodiments, R¹ and R² are each independently —OH, OC(═O)CH(CH₃)₂, or OC(═O)CH₃. In some embodiments, R¹ and R² are taken together to form —OC(═O)O—.

In some embodiments, the method comprises administering to the patient a deuterated compound of Formula A which has Formula A2

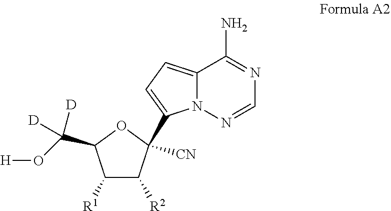

or a prodrug thereof, or a pharmaceutically acceptable salt thereof, wherein
R¹ is —OH, —OC(═O)R⁴, or —OC(═O)OR⁴;
R² is —OH, —OC(═O)R⁵, or —OC(═O)OR⁵; or
R¹ and R² are taken together to form —OC(═O)O—; and
R⁴, and R⁵ are each independently unsubstituted C₁-C₈ alkyl, unsubstituted C₃-C₈ carbocyclyl, unsubstituted C₆-C₁₀ aryl, unsubstituted 4 to 6 membered heterocyclyl containing 1, 2, or 3 heteroatoms selected from N, O, and S, or unsubstituted 5 to 6 membered heteroaryl containing 1, 2, or 3 heteroatoms selected from N, O, and S.

In some embodiments, R⁴ and R⁵ are each independently unsubstituted C₁-C₈ alkyl. In some embodiments, R⁴ and R⁵ are each independently unsubstituted C₁-C₆ alkyl. In some embodiments, R¹ is —OH or —OC(═O)R⁴ and R² is —OH or —OC(═O)R⁵. In some embodiments, R¹ and R² are each independently —OH, OC(═O)CH(CH₃)₂, or OC(═O)CH₃. In some embodiments, R¹ and R² are taken together to form —OC(═O)O—.

In some embodiments, the method comprises administering to the patient a compound of Formula I, or a pharmaceutically acceptable salt thereof, wherein
R¹ is —OH, —OC(═O)R⁴, or —OC(═O)OR⁴;
R² is —OH, —OC(═O)R⁵, or —OC(═O)OR⁵; or
R¹ and R² are taken together to form —OC(═O)O—;
R³ is —C(═O)OR⁷ or —C(═O)R⁷; and
R⁴, R⁵, and R⁷ are each independently unsubstituted C₁-C₈ alkyl, unsubstituted C₃-C₈ carbocyclyl, unsubstituted C₆-C₁₀ aryl, unsubstituted 4 to 6 membered heterocyclyl containing 1, 2, or 3 heteroatoms selected from N, O, and S, or unsubstituted 5 to 6 membered heteroaryl containing 1, 2, or 3 heteroatoms selected from N, O, and S.

In some embodiments, Base is

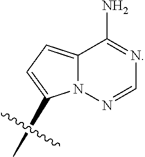

In some embodiments, R⁴, R⁵, and R⁷ are each independently unsubstituted C₁-C₈ alkyl. In some embodiments, R⁴, R⁵, and R⁷ are each independently unsubstituted C₁-C₆ alkyl. In some embodiments, R¹ is —OH or —OC(═O)R⁴, R² is —OH or —OC(═O)R⁵, and R³ is —C(═O)R⁷. In some embodiments, R¹ and R² are each independently —OH, OC(═O)CH(CH₃)₂, or OC(═O)CH₃, and R⁷ is —CH(CH₃)₂ or —CH₃. In some embodiments, R¹ and R² are taken together to form —OC(═O)O—, and R⁷ is —CH(CH₃)₂ or —CH₃.

In some embodiments, the method comprises administering to the patient a deuterated compound of Formula I which has Formula Ia

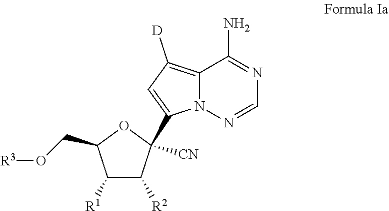

or a pharmaceutically acceptable salt thereof, wherein
R¹ is —OH, —OC(═O)R⁴, or —OC(═O)OR⁴;
R² is —OH, —OC(═O)R⁵, or —OC(═O)OR⁵; or
R¹ and R² are taken together to form —OC(═O)O—;
R³ is —C(═O)OR⁷ or —C(═O)R⁷; and
R⁴, R⁵, and R⁷ are each independently unsubstituted C₁-C₈ alkyl, unsubstituted C₃-C₈ carbocyclyl, unsubstituted C₆-C₁₀ aryl, unsubstituted 4 to 6 membered heterocyclyl containing 1, 2, or 3 heteroatoms selected from N, O, and S, or unsubstituted 5 to 6 membered heteroaryl containing 1, 2, or 3 heteroatoms selected from N, O, and S.

In some embodiments, R⁴, R⁵, and R⁷ are each independently unsubstituted C₁-C₈ alkyl. In some embodiments, R⁴, R⁵, and R⁷ are each independently unsubstituted C₁-C₆ alkyl. In some embodiments, R¹ is —OH or —OC(═O)R⁴, R² is —OH or —OC(═O)R⁵, and R³ is —C(═O)R⁷. In some embodiments, R¹ and R² are each independently —OH, OC(═O)CH(CH₃)₂, or OC(═O)CH₃, and R⁷ is —CH(CH₃)₂ or —CH₃. In some embodiments, R¹ and R² are taken together to form —OC(═O)O—, and R⁷ is —CH(CH₃)₂ or —CH₃.

In some embodiments, the method comprises administering to the patient a deuterated compound of Formula I which has Formula Ib

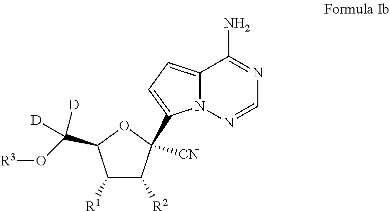

or a pharmaceutically acceptable salt thereof, wherein
R¹ is —OH, —OC(═O)R⁴, or —OC(═O)OR⁴;
R² is —OH, —OC(═O)R⁵, or —OC(═O)OR⁵; or
R¹ and R² are taken together to form —OC(═O)O—;
R³ is —C(═O)OR⁷ or —C(═O)R⁷; and
R⁴, R⁵, and R⁷ are each independently unsubstituted C₁-C₈ alkyl, unsubstituted C₃-C₈ carbocyclyl, unsubstituted C₆-C₁₀ aryl, unsubstituted 4 to 6 membered heterocyclyl containing 1, 2, or 3 heteroatoms selected from N, O, and S, or unsubstituted 5 to 6 membered heteroaryl containing 1, 2, or 3 heteroatoms selected from N, O, and S.

In some embodiments, R⁴, R⁵, and R⁷ are each independently unsubstituted C₁-C₈ alkyl. In some embodiments, R⁴, R⁵, and R⁷ are each independently unsubstituted C₁-C₆ alkyl. In some embodiments, R¹ is —OH or —OC(═O)R⁴, R² is —OH or —OC(═O)R⁵, and R³ is —C(═O)R⁷. In some embodiments, R¹ and R² are each independently —OH, OC(═O)CH(CH₃)₂, or OC(═O)CH₃, and R⁷ is —CH(CH₃)₂ or —CH₃. In some embodiments, R¹ and R² are taken together to form —OC(═O)O—, and R⁷ is —CH(CH₃)₂ or —CH₃.

Also provided herein is a compound of Formula A:

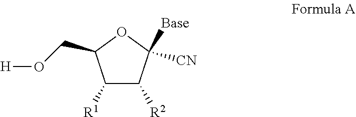

a deuterated compound of Formula A, a prodrug of the compound of Formula A, a prodrug of the deuterated compound of Formula A, or a pharmaceutically acceptable salt thereof, for use in a method of treating a viral infection in a patient in need thereof, wherein:
R¹ is —OH, —OC(═O)R⁴, or —OC(═O)OR⁴;
R² is —OH, —OC(═O)R⁵, or —OC(═O)OR⁵; or
R¹ and R² are taken together to form —OC(═O)O—, —OP(═O)(OH)O—, or —OCHR⁶O—;
R⁴ and R⁵ are each independently H, C₁-C₈ alkyl, C₂-C₈ alkenyl, C₂-C₈ alkynyl, C₃-C₈ carbocyclyl, C₆-C₁₀ aryl, 4 to 6 membered heterocyclyl containing 1, 2, or 3 heteroatoms selected from N, O, and S, or 5 to 6 membered heteroaryl containing 1, 2, or 3 heteroatoms selected from N, O, and S; wherein each C₁-C₈ alkyl, C₂-C₈ alkenyl, C₂-C₈ alkynyl, C₃-C₈ carbocyclyl, C₆-C₁₀ aryl, 4 to 6 membered heterocyclyl, and 5 to 6 membered heteroaryl of R⁴ and R⁵ is independently, optionally substituted with one, two, or three R^{a} substituents;
R⁶ is H, C₁-C₆ alkyl, C₁-C₆ alkoxy, 5 to 6 membered heteroaryl containing 1, 2, or 3 heteroatoms selected from N, O, and S, or C₆-C₁₀ aryl; wherein each 5 to 6 membered heteroaryl and C₆-C₁₀ aryl of R⁶ is independently, optionally substituted with one, two, or three R^{b} substituents;
Base is

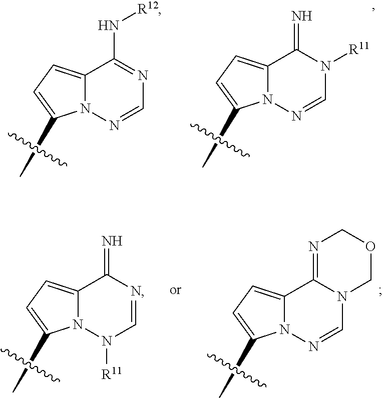

R¹¹ is C₁-C₆ alkyl optionally substituted with —OP(═O)(OH)(OR¹⁴);
R¹² is H, C₁-C₆ alkyl, —C(═O)R¹³ or —C(═O)OR¹³;
each R¹³ is independently H, C₁-C₁₀ alkyl, C₆-C₁₀ aryl, —O—C₆-C₁₀ aryl, or —O—C₁-C₁₀ alkyl; wherein each C₁-C₁₀ alkyl, C₆-C₁₀ aryl, —O—C₆-C₁₀ aryl, and —O—C₁-C₁₀ alkyl of R¹³ is independently, optionally substituted with one, two, or three R^{c} substituents;
each R^{a} is independently halo, cyano, C₁-C₆ alkyl, carbonyl, —N₃, —OR, —NR⁹R¹⁰, —OP(═O)(OH)₂, C₃-C₈ carbocyclyl, 4 to 6 membered heterocyclyl containing 1, 2, or 3 heteroatoms selected from N, O, and S, 5 to 6 membered heteroaryl containing 1, 2, or 3 heteroatoms selected from N, O, and S, or phenyl; wherein each 4 to 6 membered heterocyclyl of R^{a} is independently, optionally substituted with one, two, or three R^{d} substituents; wherein each phenyl of R^{a} is independently, optionally substituted with one, two or three R^{e} substituents;
R⁸, R⁹, and R¹⁰ are each independently H, C₁-C₆ alkyl, C₁-C₆ haloalkyl, or C₃-C₆ cycloalkyl
each R^{b} is independently halo, cyano, C₁-C₆ alkoxy, or C₁-C₆ alkyl;
each R^{c} is independently halo, cyano, —OP(═O)(OH)(OR¹⁴) or phenyl; wherein each phenyl of R^{c} is optionally substituted with —OP(═O)(OH)(OR¹⁴);
each R¹⁴ is independently H, C₁-C₈ alkyl, C₃-C₈ carbocyclyl, C₆-C₁₀ aryl, or 5 to 6 membered heteroaryl containing 1, 2, or 3 heteroatoms selected from N, O, and S; wherein each C₁-C₈ alkyl of R⁴ is independently, optionally substituted with one, two or three R^{f} substituents;
each R^{d} is independently carbonyl or C₁-C₆ alkyl;
each R^{e} is independently halo, cyano, or C₁-C₆ alkyl; and
each R^{f} is independently halo, cyano, or phenyl;
wherein when the prodrug of the compound of Formula A, the prodrug of the deuterated compound of Formula A, or the pharmaceutically acceptable salt thereof is administered, the prodrug of the compound of Formula A, the prodrug of the deuterated compound of Formula A, or the pharmaceutically acceptable salt thereof is converted substantially to the compound of Formula A or the deuterated compound of Formula A; and
wherein administering the compound of Formula A, the deuterated compound of Formula A, the prodrug of Formula A, the prodrug of the deuterated compound of Formula A, or a pharmaceutically acceptable salt thereof, to the patient results in a mean Cₘₐₓ of less than 7,000 ng/mL of the compound of Formula A, or the deuterated compound of Formula A.

Also provided herein is the use of a compound of Formula A:

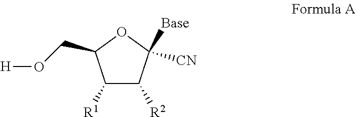

a deuterated compound of Formula A, a prodrug of the compound of Formula A, a prodrug of the deuterated compound of Formula A, or a pharmaceutically acceptable salt thereof, for the manufacture of a medicament for the treatment a viral infection in a patient in need thereof, wherein:
R¹ is —OH, —OC(═O)R⁴, or —OC(═O)OR⁴;
R² is —OH, —OC(═O)R⁵, or —OC(═O)OR⁵; or
R¹ and R² are taken together to form —OC(═O)O—, —OP(═O)(OH)O—, or —OCHR⁶O—;
R⁴ and R⁵ are each independently H, C₁-C₈ alkyl, C₂-C₈ alkenyl, C₂-C₈ alkynyl, C₃-C₈ carbocyclyl, C₆-C₁₀ aryl, 4 to 6 membered heterocyclyl containing 1, 2, or 3 heteroatoms selected from N, O, and S, or 5 to 6 membered heteroaryl containing 1, 2, or 3 heteroatoms selected from N, O, and S; wherein each C₁-C₈ alkyl, C₂-C₈ alkenyl, C₂-C₈ alkynyl, C₃-C₈ carbocyclyl, C₆-C₁₀ aryl, 4 to 6 membered heterocyclyl, and 5 to 6 membered heteroaryl of R⁴ and R is independently, optionally substituted with one, two, or three R^{a} substituents;
R⁶ is H, C₁-C₆ alkyl, C₁-C₆ alkoxy, 5 to 6 membered heteroaryl containing 1, 2, or 3 heteroatoms selected from N, O, and S, or C₆-C₁₀ aryl; wherein each 5 to 6 membered heteroaryl and C₆-C₁₀ aryl of R⁶ is independently, optionally substituted with one, two, or three R^{b} substituents;
Base is

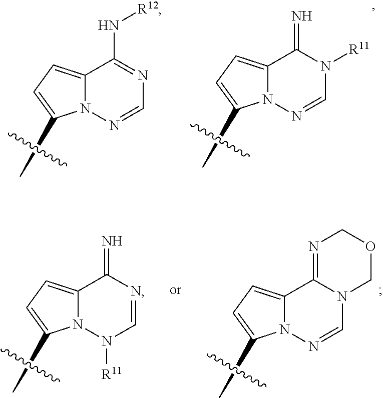

R¹¹ is C₁-C₆ alkyl optionally substituted with —OP(═O)(OH)(OR¹⁴);
R¹² is H, C₁-C₆ alkyl, —C(═O)R¹³ or —C(═O)OR¹³;
each R¹³ is independently H, C₁-C₁₀ alkyl, C₆-C₁₀ aryl, —O—C₆-C₁₀ aryl, or —O—C₁-C₁₀ alkyl; wherein each C₁-C₁₀ alkyl, C₆-C₁₀ aryl, —O—C₆-C₁₀ aryl, and —O—C₁-C₁₀ alkyl of R^{e} is independently, optionally substituted with one, two, or three R^{e} substituents;
each R^{a} is independently halo, cyano, C₁-C₆ alkyl, carbonyl, —N₃, —OR⁸, —NR⁹R¹⁰, —OP(═O)(OH)₂, C₃-C₈ carbocyclyl, 4 to 6 membered heterocyclyl containing 1, 2, or 3 heteroatoms selected from N, O, and S, 5 to 6 membered heteroaryl containing 1, 2, or 3 heteroatoms selected from N, O, and S, or phenyl; wherein each 4 to 6 membered heterocyclyl of R^{a} is independently, optionally substituted with one, two, or three R^{d} substituents; wherein each phenyl of R^{a} is independently, optionally substituted with one, two or three R^{e} substituents;
R⁸, R⁹, and R¹⁰ are each independently H, C₁-C₆ alkyl, C₁-C₆ haloalkyl, or C₃-C₆ cycloalkyl
each R^{b} is independently halo, cyano, C₁-C₆ alkoxy, or C₁-C₆ alkyl;
each R^{c} is independently halo, cyano, —OP(═O)(OH)(OR¹⁴) or phenyl; wherein each phenyl of R^{c} is optionally substituted with —OP(═O)(OH)(OR¹⁴);
each R¹⁴ is independently H, C₁-C₈ alkyl, C₃-C₈ carbocyclyl, C₆-C₁₀ aryl, or 5 to 6 membered heteroaryl containing 1, 2, or 3 heteroatoms selected from N, O, and S; wherein each C₁-C₈ alkyl of R¹⁴ is independently, optionally substituted with one, two or three R^{f} substituents;
each R^{d} is independently carbonyl or C₁-C₈ alkyl;
each R^{e} is independently halo, cyano, or C₁-C₆ alkyl; and
each R^{f} is independently halo, cyano, or phenyl;
wherein when the prodrug of the compound of Formula A, the prodrug of the deuterated compound of Formula A, or the pharmaceutically acceptable salt thereof is administered, the prodrug of the compound of Formula A, the prodrug of the deuterated compound of Formula A, or the pharmaceutically acceptable salt thereof is converted substantially to the compound of Formula A or the deuterated compound of Formula A; and
wherein administering the compound of Formula A, the deuterated compound of Formula A, the prodrug of Formula A, the prodrug of the deuterated compound of Formula A, or a pharmaceutically acceptable salt thereof, to the patient results in a mean Cₘₐₓ of less than 7,000 ng/mL of the compound of Formula A, or the deuterated compound of Formula A.

In some embodiments, the compound of Formula A or the prodrug of the compound of Formula A is a compound of Table 1, or a pharmaceutically acceptable salt thereof.

TABLE 1

| | |
|---|---|
| Compound 1 | 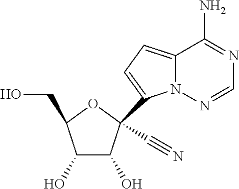 [SMILES: N#C[C@@]1(c2ccc3c(N)ncnn23)O[C@H](CO)[C@@H](O)[C@H]1O] |
| Compound 2 | 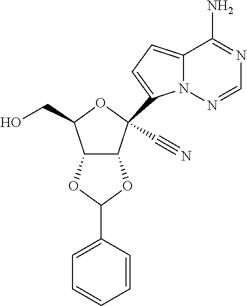 |
| Compound 3 | 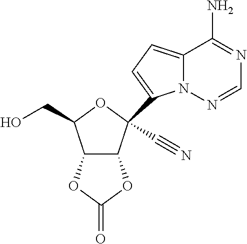 [SMILES: N#C[C@@]1(c2ccc3c(N)ncnn23)O[C@H](CO)[C@H]2OC(=O)O[C@H]21] |

In some embodiments, the compound of Formula I is a compound of Table 2, or a pharmaceutically acceptable salt thereof.

TABLE 2

| | |
|---|---|
| Compound 4 | 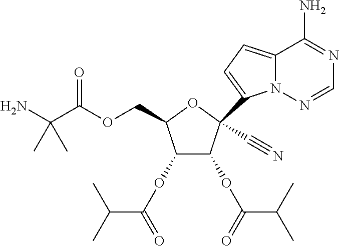 [SMILES: CC(C)C(=O)O[C@H]1[C@@H](OC(=O)C(C)C)[C@](C#N)(c2ccc3c(N)ncnn23)O[C@@H]1COC(=O)C(C)(C)N] |
| Compound 5 | 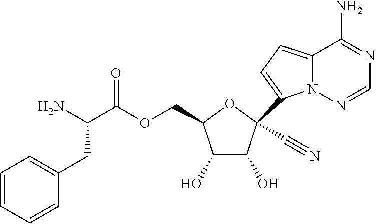 [SMILES: N#C[C@@]1(c2ccc3c(N)ncnn23)O[C@H](COC(=O)[C@@H](N)Cc2ccccc2)[C@@H](O)[C@H]1O] |
| Compound 6 | 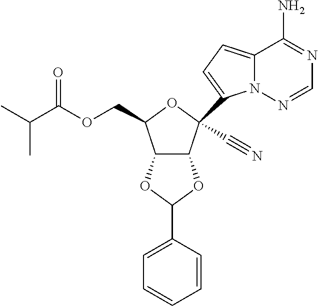 |
| Compound 7 | 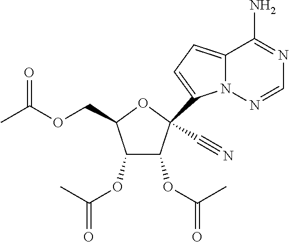 [SMILES: CC(=O)OC[C@H]1O[C@@](C#N)(c2ccc3c(N)ncnn23)[C@H](OC(C)=O)[C@@H]1OC(C)=O] |
| Compound 8 | 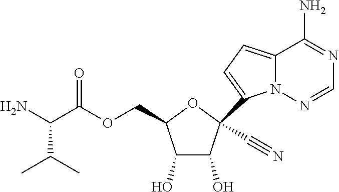 [SMILES: CC(C)[C@H](N)C(=O)OC[C@H]1O[C@@](C#N)(c2ccc3c(N)ncnn23)[C@H](O)[C@@H]1O] |
| Compound 9 | 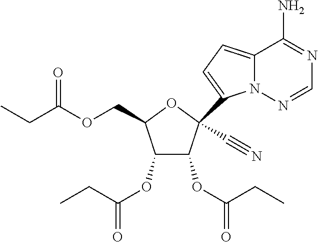 [SMILES: CCC(=O)OC[C@H]1O[C@@](C#N)(c2ccc3c(N)ncnn23)[C@H](OC(=O)CC)[C@@H]1OC(=O)CC] |
| Compound 10 | 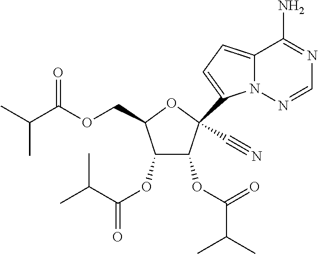 [SMILES: CC(C)C(=O)OC[C@H]1O[C@@](C#N)(c2ccc3c(N)ncnn23)[C@H](OC(=O)C(C)C)[C@@H]1OC(=O)C(C)C] |
| Compound 11 | 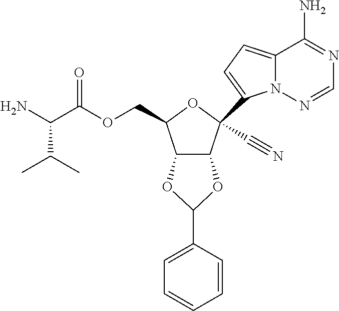 |
| Compound 12 | 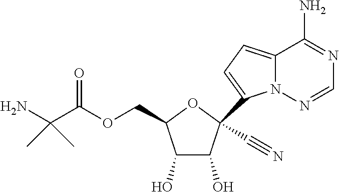 [SMILES: CC(C)(N)C(=O)OC[C@H]1O[C@@](C#N)(c2ccc3c(N)ncnn23)[C@H](O)[C@@H]1O] |
| Compound 13 | 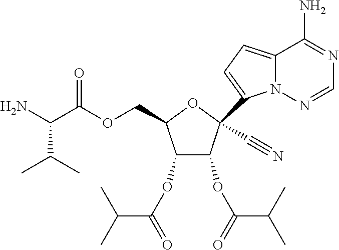 [SMILES: CC(C)C(=O)O[C@H]1[C@@H](OC(=O)C(C)C)[C@](C#N)(c2ccc3c(N)ncnn23)O[C@@H]1COC(=O)[C@@H](N)C(C)C] |
| Compound 14 | 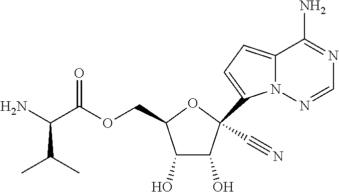 [SMILES: CC(C)[C@@H](N)C(=O)OC[C@H]1O[C@@](C#N)(c2ccc3c(N)ncnn23)[C@H](O)[C@@H]1O] |
| Compound 15 | 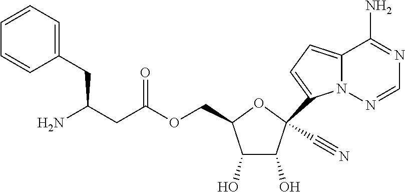 [SMILES: N#C[C@@]1(c2ccc3c(N)ncnn23)O[C@H](COC(=O)C[C@@H](N)Cc2ccccc2)[C@@H](O)[C@H]1O] |
| Compound 16 | 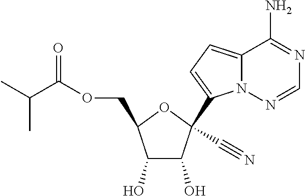 [SMILES: CC(C)C(=O)OC[C@H]1O[C@@](C#N)(c2ccc3c(N)ncnn23)[C@H](O)[C@@H]1O] |
| Compound 17 | 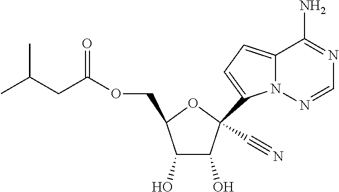 [SMILES: CC(C)CC(=O)OC[C@H]1O[C@@](C#N)(c2ccc3c(N)ncnn23)[C@H](O)[C@@H]1O] |
| Compound 18 | 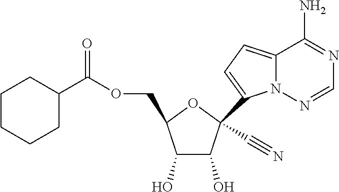 [SMILES: N#C[C@@]1(c2ccc3c(N)ncnn23)O[C@H](COC(=O)C2CCCCC2)[C@@H](O)[C@H]1O] |
| Compound 19 | 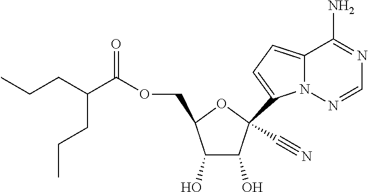 [SMILES: CCCC(CCC)C(=O)OC[C@H]1O[C@@](C#N)(c2ccc3c(N)ncnn23)[C@H](O)[C@@H]1O] |
| Compound 20 | 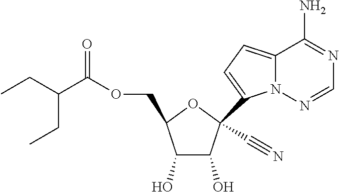 [SMILES: CCC(CC)C(=O)OC[C@H]1O[C@@](C#N)(c2ccc3c(N)ncnn23)[C@H](O)[C@@H]1O] |
| Compound 21 | 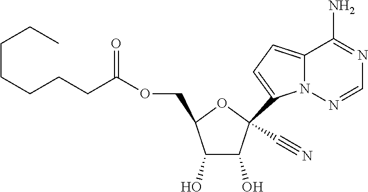 [SMILES: CCCCCCCC(=O)OC[C@H]1O[C@@](C#N)(c2ccc3c(N)ncnn23)[C@H](O)[C@@H]1O] |
| Compound 22 | 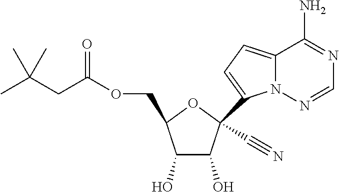 [SMILES: CC(C)(C)CC(=O)OC[C@H]1O[C@@](C#N)(c2ccc3c(N)ncnn23)[C@H](O)[C@@H]1O] |
| Compound 23 | 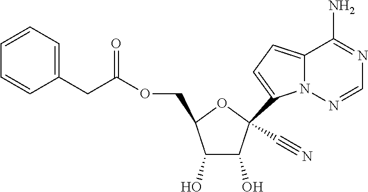 [SMILES: N#C[C@@]1(c2ccc3c(N)ncnn23)O[C@H](COC(=O)Cc2ccccc2)[C@@H](O)[C@H]1O] |
| Compound 24 | 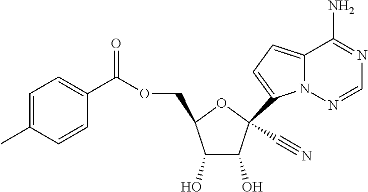 [SMILES: Cc1ccc(C(=O)OC[C@H]2O[C@@](C#N)(c3ccc4c(N)ncnn34)[C@H](O)[C@@H]2O)cc1] |
| Compound 25 | 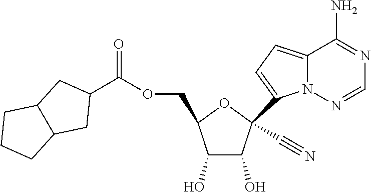 [SMILES: N#C[C@@]1(c2ccc3c(N)ncnn23)O[C@H](COC(=O)C2CC3CCCC3C2)[C@@H](O)[C@H]1O] |
| Compound 26 | 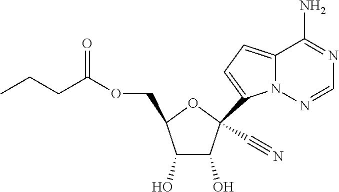 [SMILES: CCCC(=O)OC[C@H]1O[C@@](C#N)(c2ccc3c(N)ncnn23)[C@H](O)[C@@H]1O] |
| Compound 27 | 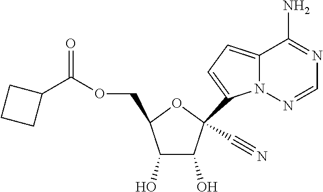 [SMILES: N#C[C@@]1(c2ccc3c(N)ncnn23)O[C@H](COC(=O)C2CCC2)[C@@H](O)[C@H]1O] |
| Compound 28 | 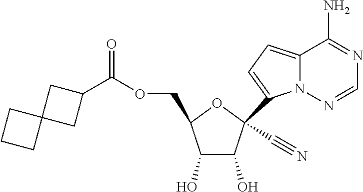 [SMILES: N#C[C@@]1(c2ccc3c(N)ncnn23)O[C@H](COC(=O)C2CC3(CCC3)C2)[C@@H](O)[C@H]1O] |
| Compound 29 | 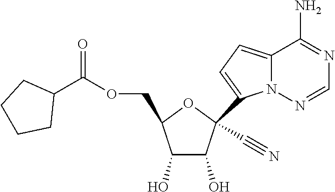 [SMILES: N#C[C@@]1(c2ccc3c(N)ncnn23)O[C@H](COC(=O)C2CCCC2)[C@@H](O)[C@H]1O] |
| Compound 30 | 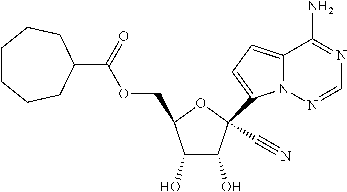 [SMILES: N#C[C@@]1(c2ccc3c(N)ncnn23)O[C@H](COC(=O)C2CCCCCC2)[C@@H](O)[C@H]1O] |
| Compound 31 | 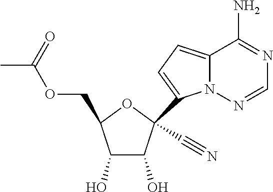 [SMILES: CC(=O)OC[C@H]1O[C@@](C#N)(c2ccc3c(N)ncnn23)[C@H](O)[C@@H]1O] |
| Compound 32 | 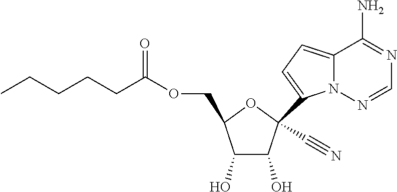 [SMILES: CCCCCC(=O)OC[C@H]1O[C@@](C#N)(c2ccc3c(N)ncnn23)[C@H](O)[C@@H]1O] |
| Compound 33 | 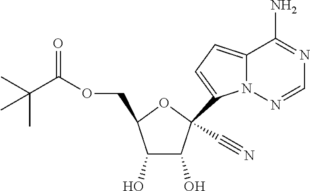 [SMILES: CC(C)(C)C(=O)OC[C@H]1O[C@@](C#N)(c2ccc3c(N)ncnn23)[C@H](O)[C@@H]1O] |
| Compound 34 | 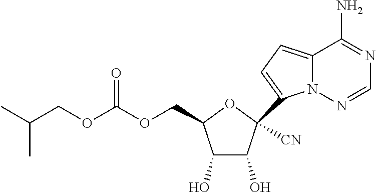 [SMILES: CC(C)COC(=O)OC[C@H]1O[C@@](C#N)(c2ccc3c(N)ncnn23)[C@H](O)[C@@H]1O] |
| Compound 35 | 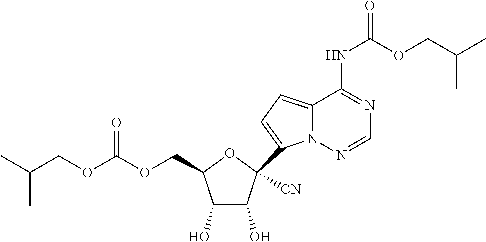 [SMILES: CC(C)COC(=O)Nc1ncnn2c([C@]3(C#N)O[C@H](COC(=O)OCC(C)C)[C@@H](O)[C@H]3O)ccc12] |
| Compound 36 | 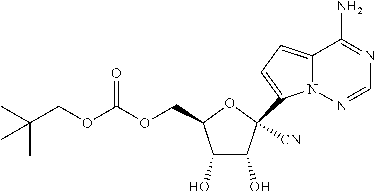 [SMILES: CC(C)(C)COC(=O)OC[C@H]1O[C@@](C#N)(c2ccc3c(N)ncnn23)[C@H](O)[C@@H]1O] |
| Compound 37 | 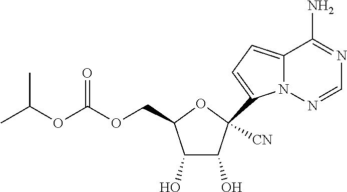 [SMILES: CC(C)OC(=O)OC[C@H]1O[C@@](C#N)(c2ccc3c(N)ncnn23)[C@H](O)[C@@H]1O] |
| Compound 38 | 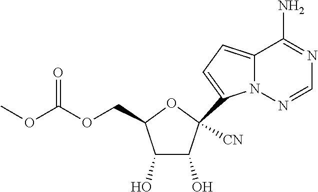 [SMILES: COC(=O)OC[C@H]1O[C@@](C#N)(c2ccc3c(N)ncnn23)[C@H](O)[C@@H]1O] |
| Compound 39 | 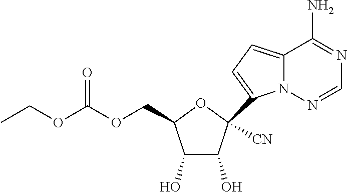 [SMILES: CCOC(=O)OC[C@H]1O[C@@](C#N)(c2ccc3c(N)ncnn23)[C@H](O)[C@@H]1O] |
| Compound 40 | 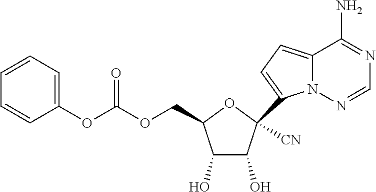 [SMILES: N#C[C@@]1(c2ccc3c(N)ncnn23)O[C@H](COC(=O)Oc2ccccc2)[C@@H](O)[C@H]1O] |
| Compound 41 | 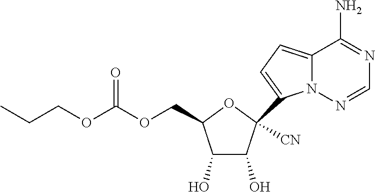 [SMILES: CCCOC(=O)OC[C@H]1O[C@@](C#N)(c2ccc3c(N)ncnn23)[C@H](O)[C@@H]1O] |
| Compound 42 | 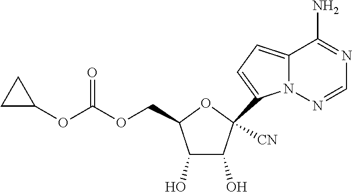 [SMILES: N#C[C@@]1(c2ccc3c(N)ncnn23)O[C@H](COC(=O)OC2CC2)[C@@H](O)[C@H]1O] |
| Compound 43 | 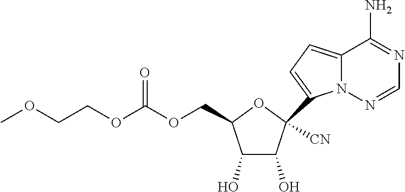 [SMILES: COCCOC(=O)OC[C@H]1O[C@@](C#N)(c2ccc3c(N)ncnn23)[C@H](O)[C@@H]1O] |
| Compound 44 | 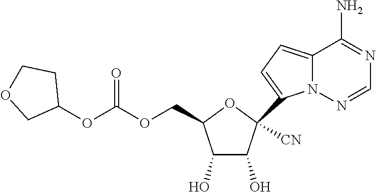 [SMILES: N#C[C@@]1(c2ccc3c(N)ncnn23)O[C@H](COC(=O)OC2CCOC2)[C@@H](O)[C@H]1O] |
| Compound 45 | 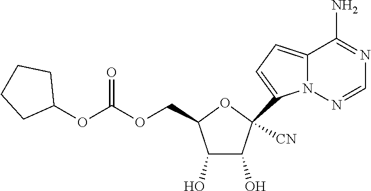 [SMILES: N#C[C@@]1(c2ccc3c(N)ncnn23)O[C@H](COC(=O)OC2CCCC2)[C@@H](O)[C@H]1O] |
| Compound 46 | 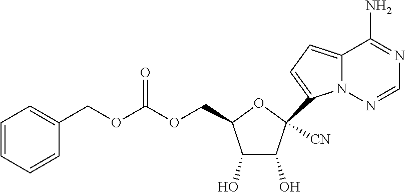 [SMILES: N#C[C@@]1(c2ccc3c(N)ncnn23)O[C@H](COC(=O)OCc2ccccc2)[C@@H](O)[C@H]1O] |
| Compound 47 | 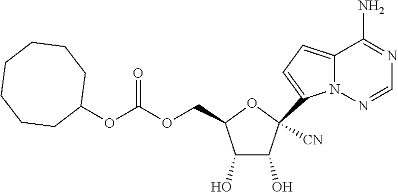 [SMILES: N#C[C@@]1(c2ccc3c(N)ncnn23)O[C@H](COC(=O)OC2CCCCCCC2)[C@@H](O)[C@H]1O] |
| Compound 48 | 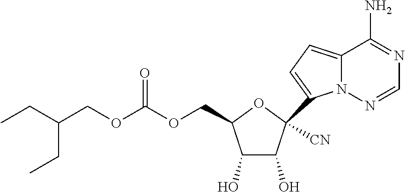 [SMILES: CCC(CC)COC(=O)OC[C@H]1O[C@@](C#N)(c2ccc3c(N)ncnn23)[C@H](O)[C@@H]1O] |
| Compound 49 | 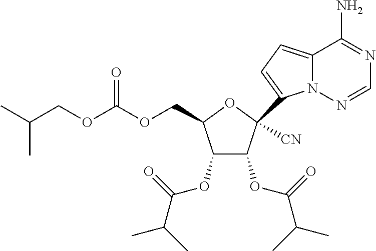 [SMILES: CC(C)COC(=O)OC[C@H]1O[C@@](C#N)(c2ccc3c(N)ncnn23)[C@H](OC(=O)C(C)C)[C@@H]1OC(=O)C(C)C] |
| Compound 50 | 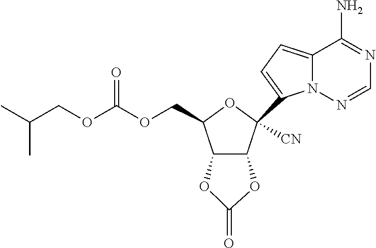 [SMILES: CC(C)COC(=O)OC[C@H]1O[C@@](C#N)(c2ccc3c(N)ncnn23)[C@@H]2OC(=O)O[C@@H]21] |
| Compound 51 | 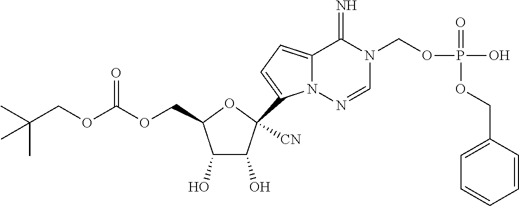 [SMILES: CC(C)(C)COC(=O)OC[C@H]1O[C@@](C#N)(c2ccc3c(=N)n(COP(=O)(O)OCc4ccccc4)cnn23)[C@H](O)[C@@H]1O] |
| Compound 52 | 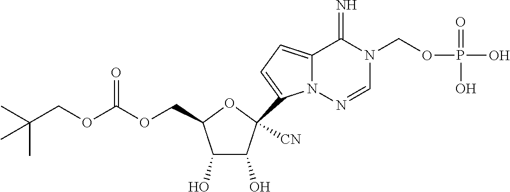 [SMILES: CC(C)(C)COC(=O)OC[C@H]1O[C@@](C#N)(c2ccc3c(=N)n(COP(=O)(O)O)cnn23)[C@H](O)[C@@H]1O] |
| Compound 53 | 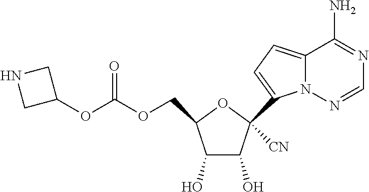 [SMILES: N#C[C@@]1(c2ccc3c(N)ncnn23)O[C@H](COC(=O)OC2CNC2)[C@@H](O)[C@H]1O] |
| Compound 54 | 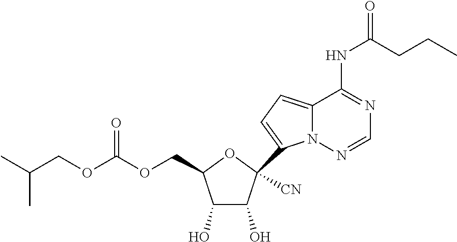 [SMILES: CCCC(=O)Nc1ncnn2c([C@]3(C#N)O[C@H](COC(=O)OCC(C)C)[C@@H](O)[C@H]3O)ccc12] |
| Compound 55 | 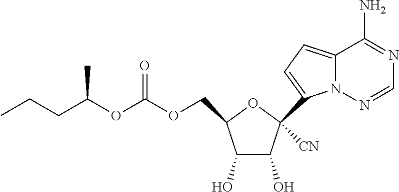 [SMILES: CCC[C@@H](C)OC(=O)OC[C@H]1O[C@@](C#N)(c2ccc3c(N)ncnn23)[C@H](O)[C@@H]1O] |
| Compound 56 | 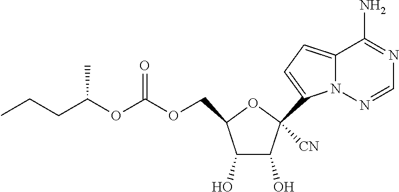 [SMILES: CCC[C@H](C)OC(=O)OC[C@H]1O[C@@](C#N)(c2ccc3c(N)ncnn23)[C@H](O)[C@@H]1O] |
| Compound 57 | 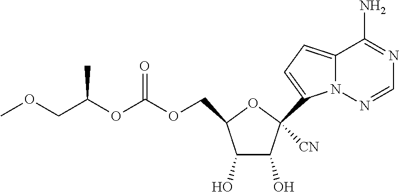 [SMILES: COC[C@@H](C)OC(=O)OC[C@H]1O[C@@](C#N)(c2ccc3c(N)ncnn23)[C@H](O)[C@@H]1O] |
| Compound 58 | 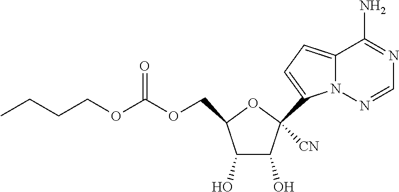 [SMILES: CCCCOC(=O)OC[C@H]1O[C@@](C#N)(c2ccc3c(N)ncnn23)[C@H](O)[C@@H]1O] |
| Compound 59 | 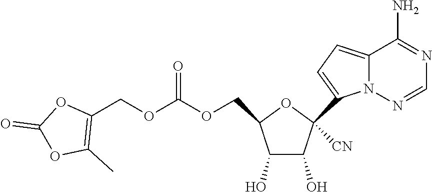 [SMILES: Cc1oc(=O)oc1COC(=O)OC[C@H]1O[C@@](C#N)(c2ccc3c(N)ncnn23)[C@H](O)[C@@H]1O] |
| Compound 60 | 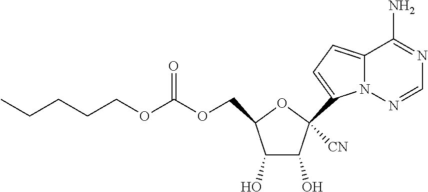 [SMILES: CCCCCOC(=O)OC[C@H]1O[C@@](C#N)(c2ccc3c(N)ncnn23)[C@H](O)[C@@H]1O] |
| Compound 61 | 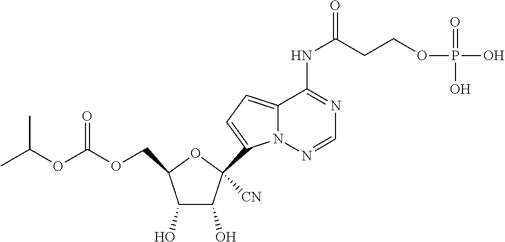 [SMILES: CC(C)OC(=O)OC[C@H]1O[C@@](C#N)(c2ccc3c(NC(=O)CCOP(=O)(O)O)ncnn23)[C@H](O)[C@@H]1O] |
| Compound 62 | 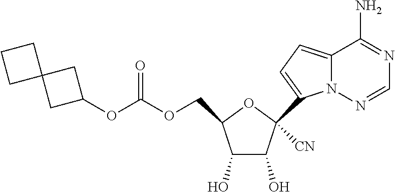 [SMILES: N#C[C@@]1(c2ccc3c(N)ncnn23)O[C@H](COC(=O)OC2CC3(CCC3)C2)[C@@H](O)[C@H]1O] |
| Compound 63 | 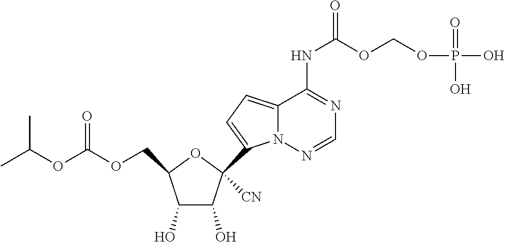 [SMILES: CC(C)OC(=O)OC[C@H]1O[C@@](C#N)(c2ccc3c(NC(=O)OCOP(=O)(O)O)ncnn23)[C@H](O)[C@@H]1O] |
| Compound 64 | 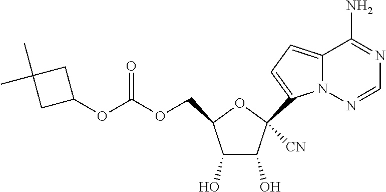 [SMILES: CC1(C)CC(OC(=O)OC[C@H]2O[C@@](C#N)(c3ccc4c(N)ncnn34)[C@H](O)[C@@H]2O)C1] |
| Compound 65 | 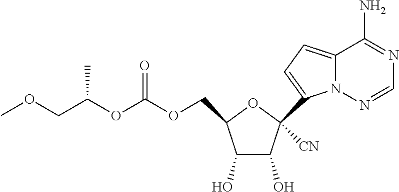 [SMILES: COC[C@H](C)OC(=O)OC[C@H]1O[C@@](C#N)(c2ccc3c(N)ncnn23)[C@H](O)[C@@H]1O] |
| Compound 66 | 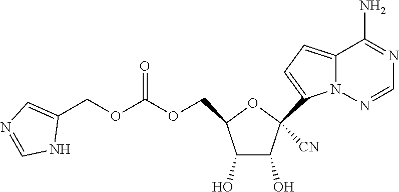 [SMILES: N#C[C@@]1(c2ccc3c(N)ncnn23)O[C@H](COC(=O)OCc2cnc[nH]2)[C@@H](O)[C@H]1O] |
| Compound 67 | 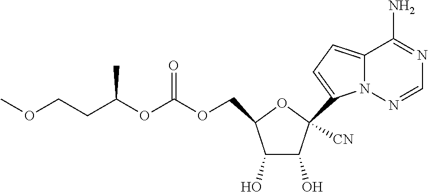 [SMILES: COCC[C@@H](C)OC(=O)OC[C@H]1O[C@@](C#N)(c2ccc3c(N)ncnn23)[C@H](O)[C@@H]1O] |
| Compound 68 | 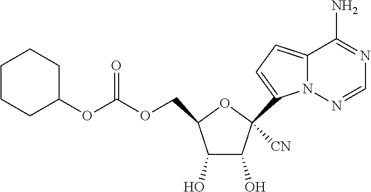 [SMILES: N#C[C@@]1(c2ccc3c(N)ncnn23)O[C@H](COC(=O)OC2CCCCC2)[C@@H](O)[C@H]1O] |
| Compound 69 | 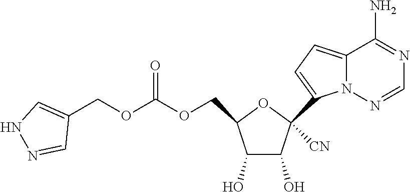 [SMILES: N#C[C@@]1(c2ccc3c(N)ncnn23)O[C@H](COC(=O)OCc2cn[nH]c2)[C@@H](O)[C@H]1O] |
| Compound 70 | 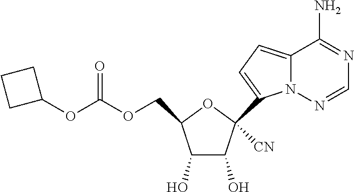 [SMILES: N#C[C@@]1(c2ccc3c(N)ncnn23)O[C@H](COC(=O)OC2CCC2)[C@@H](O)[C@H]1O] |
| Compound 71 | 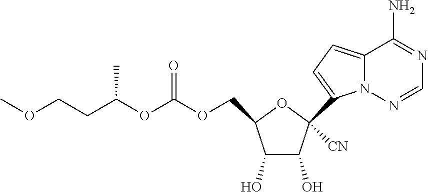 [SMILES: COCC[C@H](C)OC(=O)OC[C@H]1O[C@@](C#N)(c2ccc3c(N)ncnn23)[C@H](O)[C@@H]1O] |
| Compound 72 | 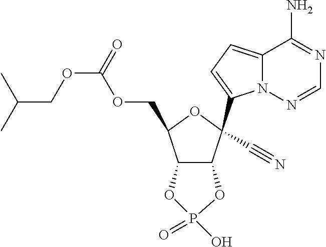 |
| Compound 73 | 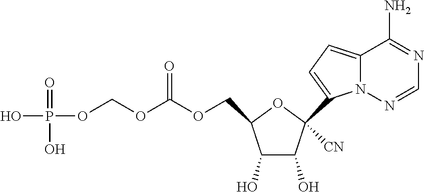 [SMILES: N#C[C@@]1(c2ccc3c(N)ncnn23)O[C@H](COC(=O)OCOP(=O)(O)O)[C@@H](O)[C@H]1O] |
| Compound 74 | 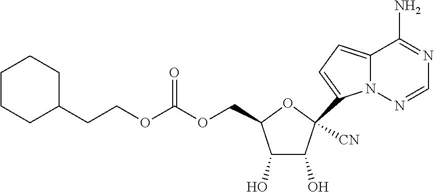 [SMILES: N#C[C@@]1(c2ccc3c(N)ncnn23)O[C@H](COC(=O)OCCC2CCCCC2)[C@@H](O)[C@H]1O] |
| Compound 75 | 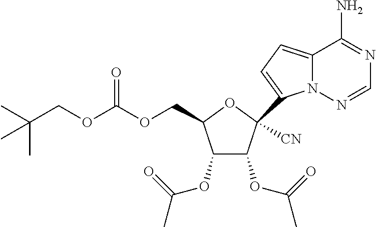 [SMILES: CC(=O)O[C@H]1[C@@H](OC(C)=O)[C@](C#N)(c2ccc3c(N)ncnn23)O[C@@H]1COC(=O)OCC(C)(C)C] |
| Compound 76 | 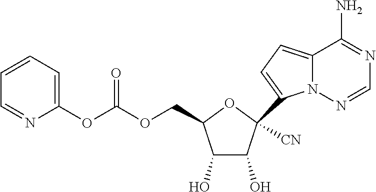 [SMILES: N#C[C@@]1(c2ccc3c(N)ncnn23)O[C@H](COC(=O)Oc2ccccn2)[C@@H](O)[C@H]1O] |
| Compound 77 | 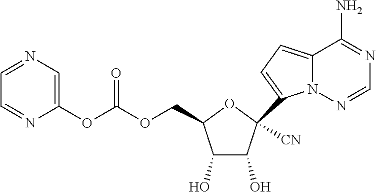 [SMILES: N#C[C@@]1(c2ccc3c(N)ncnn23)O[C@H](COC(=O)Oc2cnccn2)[C@@H](O)[C@H]1O] |
| Compound 78 | 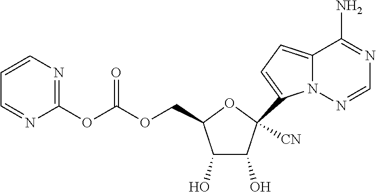 [SMILES: N#C[C@@]1(c2ccc3c(N)ncnn23)O[C@H](COC(=O)Oc2ncccn2)[C@@H](O)[C@H]1O] |
| Compound 79 | 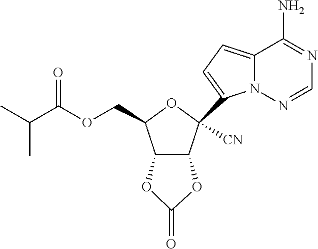 [SMILES: CC(C)C(=O)OC[C@H]1O[C@@](C#N)(c2ccc3c(N)ncnn23)[C@@H]2OC(=O)O[C@@H]21] |
| Compound 80 | 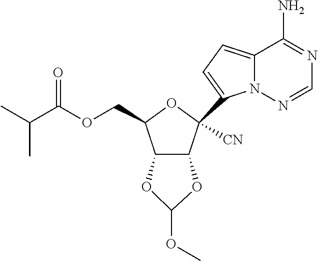 |
| Compound 81 | 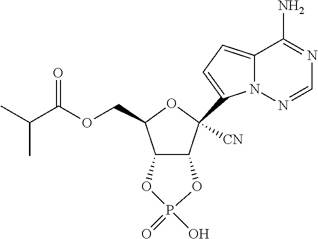 |
| Compound 82 | 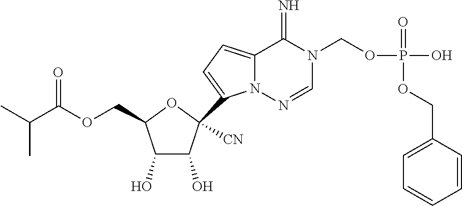 [SMILES: CC(C)C(=O)OC[C@H]1O[C@@](C#N)(c2ccc3c(=N)n(COP(=O)(O)OCc4ccccc4)cnn23)[C@H](O)[C@@H]1O] |
| Compound 83 | 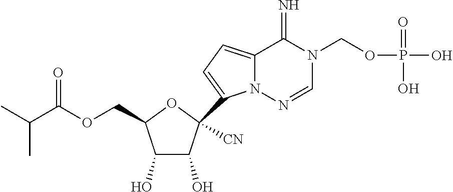 [SMILES: CC(C)C(=O)OC[C@H]1O[C@@](C#N)(c2ccc3c(=N)n(COP(=O)(O)O)cnn23)[C@H](O)[C@@H]1O] |
| Compound 84 | 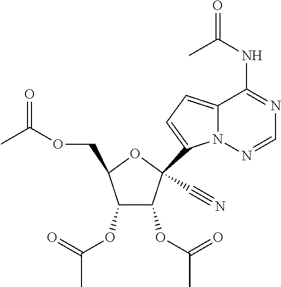 [SMILES: CC(=O)Nc1ncnn2c([C@]3(C#N)O[C@H](COC(C)=O)[C@@H](OC(C)=O)[C@H]3OC(C)=O)ccc12] |
| Compound 85 | 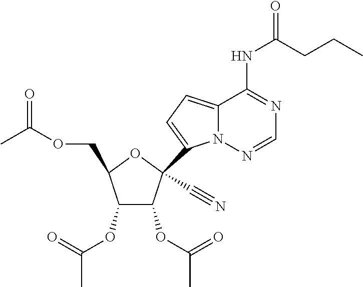 [SMILES: CCCC(=O)Nc1ncnn2c([C@]3(C#N)O[C@H](COC(C)=O)[C@@H](OC(C)=O)[C@H]3OC(C)=O)ccc12] |
| Compound 86 | 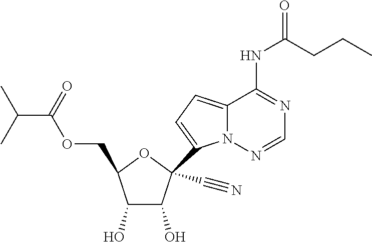 [SMILES: CCCC(=O)Nc1ncnn2c([C@]3(C#N)O[C@H](COC(=O)C(C)C)[C@@H](O)[C@H]3O)ccc12] |
| Compound 87 | 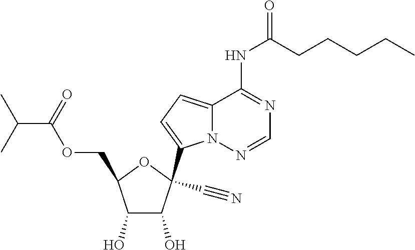 [SMILES: CCCCCC(=O)Nc1ncnn2c([C@]3(C#N)O[C@H](COC(=O)C(C)C)[C@@H](O)[C@H]3O)ccc12] |
| Compound 88 | 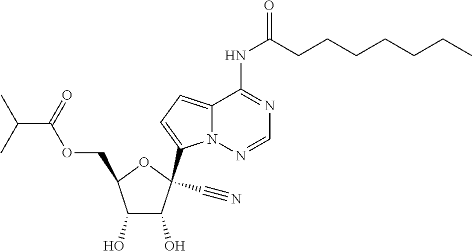 [SMILES: CCCCCCCC(=O)Nc1ncnn2c([C@]3(C#N)O[C@H](COC(=O)C(C)C)[C@@H](O)[C@H]3O)ccc12] |
| Compound 89 | 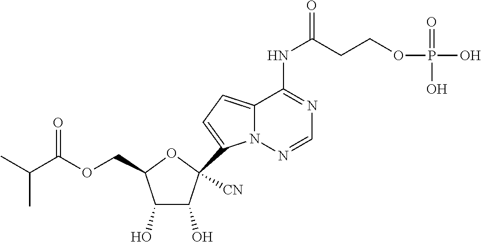 [SMILES: CC(C)C(=O)OC[C@H]1O[C@@](C#N)(c2ccc3c(NC(=O)CCOP(=O)(O)O)ncnn23)[C@H](O)[C@@H]1O] |
| Compound 90 | 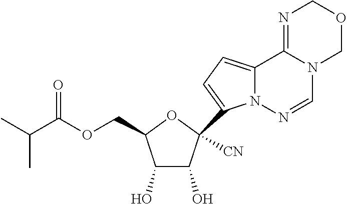 [SMILES: CC(C)C(=O)OC[C@H]1O[C@@](C#N)(c2ccc3n2N=CN2COCN=C32)[C@H](O)[C@@H]1O] |
| Compound 91 | 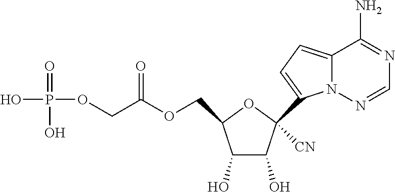 [SMILES: N#C[C@@]1(c2ccc3c(N)ncnn23)O[C@H](COC(=O)COP(=O)(O)O)[C@@H](O)[C@H]1O] |

In some embodiments, the deuterated compound of Formula A, the prodrug of the compound of Formula A, or the prodrug of the deuterated compound of Formula A is a compound of Table 3, or a pharmaceutically acceptable salt thereof.

TABLE 3

| | |
|---|---|
| Compound 92 | 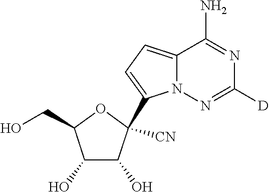 [SMILES: [2H]c1nc(N)c2ccc([C@]3(C#N)O[C@H](CO)[C@@H](O)[C@H]3O)n2n1] |
| Compound 93 | 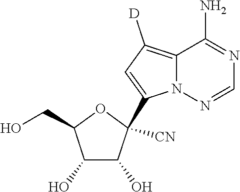 [SMILES: [2H]c1cc([C@]2(C#N)O[C@H](CO)[C@@H](O)[C@H]2O)n2ncnc(N)c12] |
| Compound 94 | 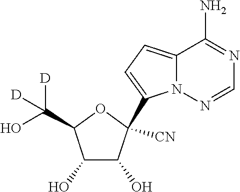 [SMILES: [2H]C([2H])(O)[C@H]1O[C@@](C#N)(c2ccc3c(N)ncnn23)[C@H](O)[C@@H]1O] |
| Compound 95 | 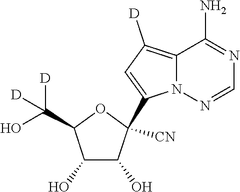 [SMILES: [2H]c1cc([C@]2(C#N)O[C@H](C([2H])([2H])O)[C@@H](O)[C@H]2O)n2ncnc(N)c12] |
| Compound 96 | 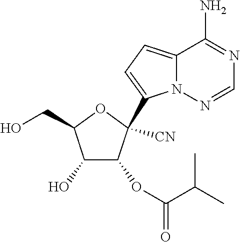 [SMILES: CC(C)C(=O)O[C@@H]1[C@H](O)[C@@H](CO)O[C@@]1(C#N)c1ccc2c(N)ncnn12] |
| Compound 97 | 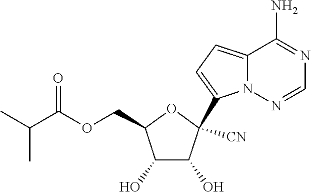 [SMILES: CC(C)C(=O)OC[C@H]1O[C@@](C#N)(c2ccc3c(N)ncnn23)[C@H](O)[C@@H]1O] |
| Compound 98 | 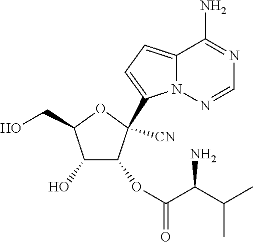 [SMILES: CC(C)[C@H](N)C(=O)O[C@@H]1[C@H](O)[C@@H](CO)O[C@@]1(C#N)c1ccc2c(N)ncnn12] |
| Compound 99 | 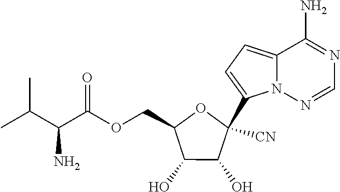 [SMILES: CC(C)[C@H](N)C(=O)OC[C@H]1O[C@@](C#N)(c2ccc3c(N)ncnn23)[C@H](O)[C@@H]1O] |
| Compound 100 | 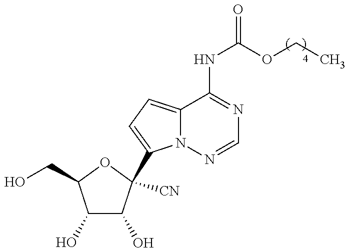 |
| Compound 101 | 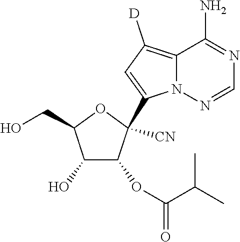 [SMILES: [2H]c1cc([C@]2(C#N)O[C@H](CO)[C@@H](O)[C@H]2OC(=O)C(C)C)n2ncnc(N)c12] |
| Compound 102 | 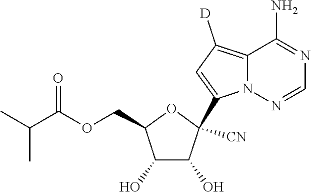 [SMILES: [2H]c1cc([C@]2(C#N)O[C@H](COC(=O)C(C)C)[C@@H](O)[C@H]2O)n2ncnc(N)c12] |
| Compound 103 | 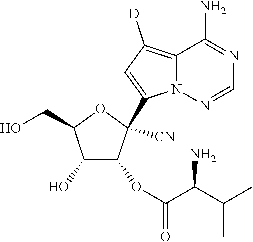 [SMILES: [2H]c1cc([C@]2(C#N)O[C@H](CO)[C@@H](O)[C@H]2OC(=O)[C@@H](N)C(C)C)n2ncnc(N)c12] |
| Compound 104 | 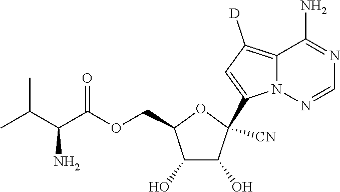 [SMILES: [2H]c1cc([C@]2(C#N)O[C@H](COC(=O)[C@@H](N)C(C)C)[C@@H](O)[C@H]2O)n2ncnc(N)c12] |
| Compound 105 | 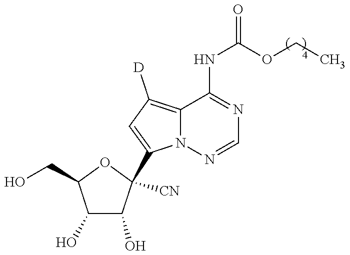 |
| Compound 106 | 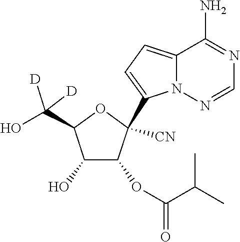 [SMILES: [2H]C([2H])(O)[C@H]1O[C@@](C#N)(c2ccc3c(N)ncnn23)[C@H](OC(=O)C(C)C)[C@@H]1O] |
| Compound 107 | 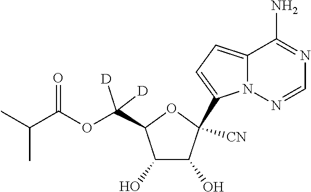 [SMILES: [2H]C([2H])(OC(=O)C(C)C)[C@H]1O[C@@](C#N)(c2ccc3c(N)ncnn23)[C@H](O)[C@@H]1O] |
| Compound 108 | 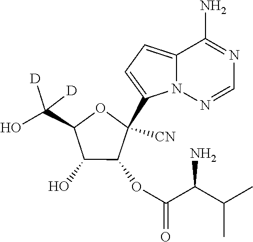 [SMILES: [2H]C([2H])(O)[C@H]1O[C@@](C#N)(c2ccc3c(N)ncnn23)[C@H](OC(=O)[C@@H](N)C(C)C)[C@@H]1O] |
| Compound 109 | 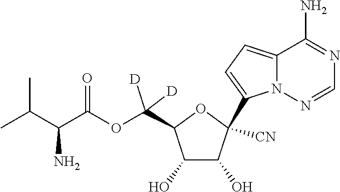 [SMILES: [2H]C([2H])(OC(=O)[C@@H](N)C(C)C)[C@H]1O[C@@](C#N)(c2ccc3c(N)ncnn23)[C@H](O)[C@@H]1O] |
| Compound 110 | 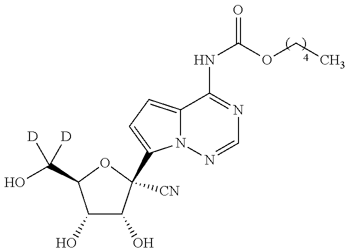 |
| Compound 111 | 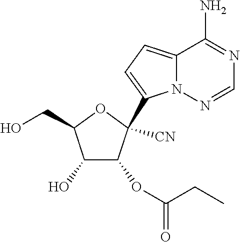 [SMILES: CCC(=O)O[C@@H]1[C@H](O)[C@@H](CO)O[C@@]1(C#N)c1ccc2c(N)ncnn12] |
| Compound 112 | 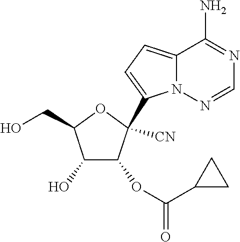 [SMILES: N#C[C@@]1(c2ccc3c(N)ncnn23)O[C@H](CO)[C@@H](O)[C@H]1OC(=O)C1CC1] |
| Compound 113 | 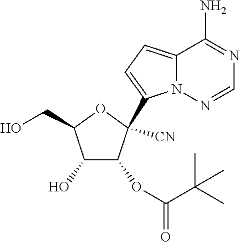 [SMILES: CC(C)(C)C(=O)O[C@@H]1[C@H](O)[C@@H](CO)O[C@@]1(C#N)c1ccc2c(N)ncnn12] |
| Compound 114 | 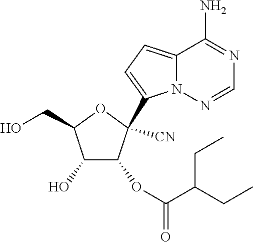 [SMILES: CCC(CC)C(=O)O[C@@H]1[C@H](O)[C@@H](CO)O[C@@]1(C#N)c1ccc2c(N)ncnn12] |
| Compound 115 | 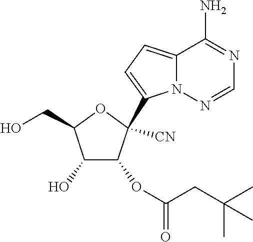 [SMILES: CC(C)(C)CC(=O)O[C@@H]1[C@H](O)[C@@H](CO)O[C@@]1(C#N)c1ccc2c(N)ncnn12] |
| Compound 116 | 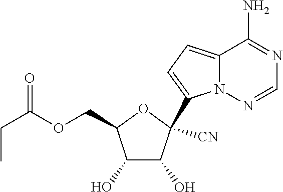 [SMILES: CCC(=O)OC[C@H]1O[C@@](C#N)(c2ccc3c(N)ncnn23)[C@H](O)[C@@H]1O] |
| Compound 117 | 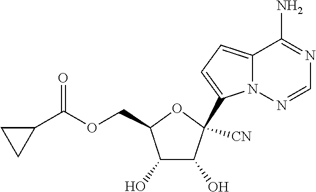 [SMILES: N#C[C@@]1(c2ccc3c(N)ncnn23)O[C@H](COC(=O)C2CC2)[C@@H](O)[C@H]1O] |
| Compound 118 | 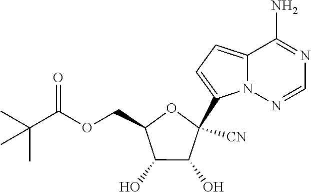 [SMILES: CC(C)(C)C(=O)OC[C@H]1O[C@@](C#N)(c2ccc3c(N)ncnn23)[C@H](O)[C@@H]1O] |
| Compound 119 | 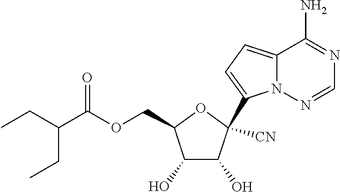 [SMILES: CCC(CC)C(=O)OC[C@H]1O[C@@](C#N)(c2ccc3c(N)ncnn23)[C@H](O)[C@@H]1O] |
| Compound 120 | 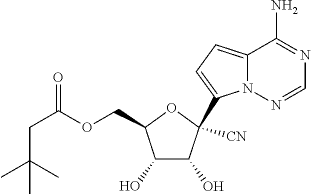 [SMILES: CC(C)(C)CC(=O)OC[C@H]1O[C@@](C#N)(c2ccc3c(N)ncnn23)[C@H](O)[C@@H]1O] |
| Compound 121 | 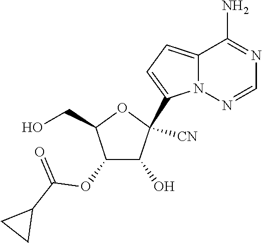 [SMILES: N#C[C@@]1(c2ccc3c(N)ncnn23)O[C@H](CO)[C@@H](OC(=O)C2CC2)[C@H]1O] |
| Compound 122 | 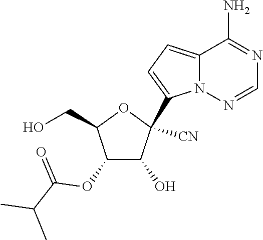 [SMILES: CC(C)C(=O)O[C@H]1[C@@H](O)[C@](C#N)(c2ccc3c(N)ncnn23)O[C@@H]1CO] |
| Compound 123 | 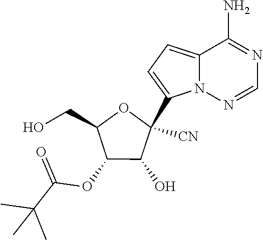 [SMILES: CC(C)(C)C(=O)O[C@H]1[C@@H](O)[C@](C#N)(c2ccc3c(N)ncnn23)O[C@@H]1CO] |
| Compound 124 | 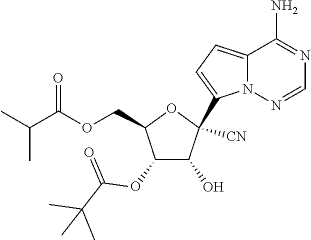 [SMILES: CC(C)C(=O)OC[C@H]1O[C@@](C#N)(c2ccc3c(N)ncnn23)[C@H](O)[C@@H]1OC(=O)C(C)(C)C] |
| Compound 125 | 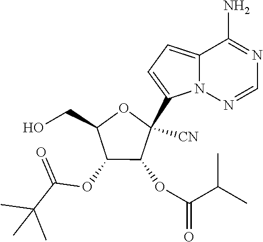 [SMILES: CC(C)C(=O)O[C@@H]1[C@H](OC(=O)C(C)(C)C)[C@@H](CO)O[C@@]1(C#N)c1ccc2c(N)ncnn12] |
| Compound 126 | 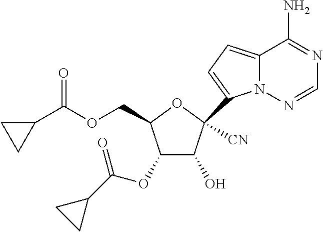 [SMILES: N#C[C@@]1(c2ccc3c(N)ncnn23)O[C@H](COC(=O)C2CC2)[C@@H](OC(=O)C2CC2)[C@H]1O] |
| Compound 127 | 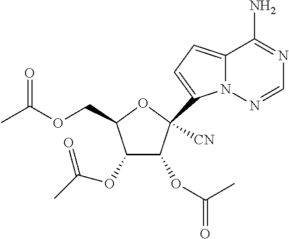 [SMILES: CC(=O)OC[C@H]1O[C@@](C#N)(c2ccc3c(N)ncnn23)[C@H](OC(C)=O)[C@@H]1OC(C)=O] |
| Compound 128 | 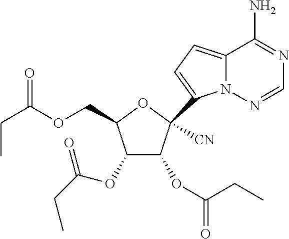 [SMILES: CCC(=O)OC[C@H]1O[C@@](C#N)(c2ccc3c(N)ncnn23)[C@H](OC(=O)CC)[C@@H]1OC(=O)CC] |
| Compound 129 | 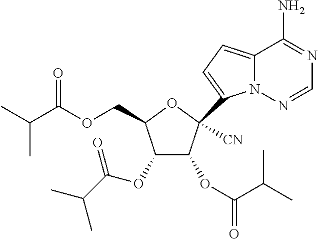 [SMILES: CC(C)C(=O)OC[C@H]1O[C@@](C#N)(c2ccc3c(N)ncnn23)[C@H](OC(=O)C(C)C)[C@@H]1OC(=O)C(C)C] |
| Compound 130 | 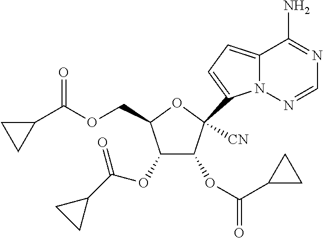 [SMILES: N#C[C@@]1(c2ccc3c(N)ncnn23)O[C@H](COC(=O)C2CC2)[C@@H](OC(=O)C2CC2)[C@H]1OC(=O)C1CC1] |
| Compound 131 | 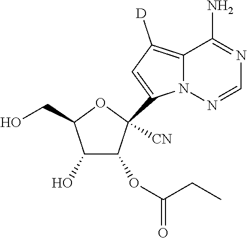 [SMILES: [2H]c1cc([C@]2(C#N)O[C@H](CO)[C@@H](O)[C@H]2OC(=O)CC)n2ncnc(N)c12] |
| Compound 132 | 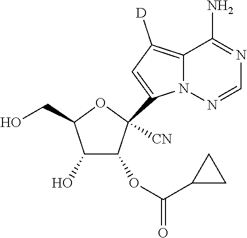 [SMILES: [2H]c1cc([C@]2(C#N)O[C@H](CO)[C@@H](O)[C@H]2OC(=O)C2CC2)n2ncnc(N)c12] |
| Compound 133 | 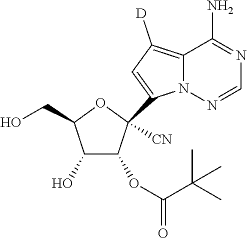 [SMILES: [2H]c1cc([C@]2(C#N)O[C@H](CO)[C@@H](O)[C@H]2OC(=O)C(C)(C)C)n2ncnc(N)c12] |
| Compound 134 | 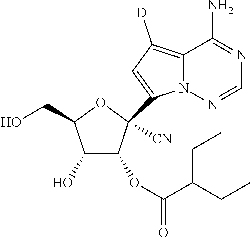 [SMILES: [2H]c1cc([C@]2(C#N)O[C@H](CO)[C@@H](O)[C@H]2OC(=O)C(CC)CC)n2ncnc(N)c12] |
| Compound 135 | 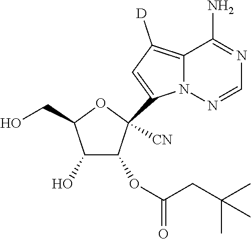 [SMILES: [2H]c1cc([C@]2(C#N)O[C@H](CO)[C@@H](O)[C@H]2OC(=O)CC(C)(C)C)n2ncnc(N)c12] |
| Compound 136 | 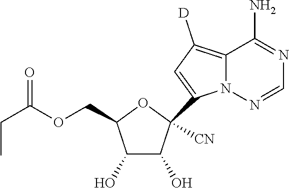 [SMILES: [2H]c1cc([C@]2(C#N)O[C@H](COC(=O)CC)[C@@H](O)[C@H]2O)n2ncnc(N)c12] |
| Compound 137 | 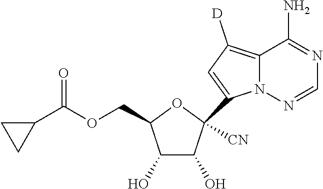 [SMILES: [2H]c1cc([C@]2(C#N)O[C@H](COC(=O)C3CC3)[C@@H](O)[C@H]2O)n2ncnc(N)c12] |
| Compound 138 | 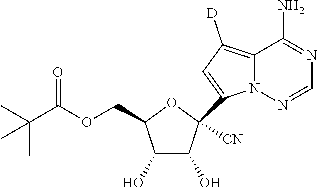 [SMILES: [2H]c1cc([C@]2(C#N)O[C@H](COC(=O)C(C)(C)C)[C@@H](O)[C@H]2O)n2ncnc(N)c12] |
| Compound 139 | 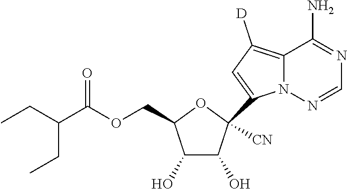 [SMILES: [2H]c1cc([C@]2(C#N)O[C@H](COC(=O)C(CC)CC)[C@@H](O)[C@H]2O)n2ncnc(N)c12] |
| Compound 140 | 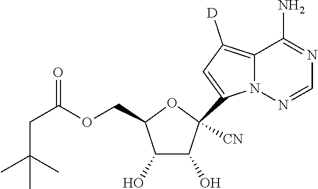 [SMILES: [2H]c1cc([C@]2(C#N)O[C@H](COC(=O)CC(C)(C)C)[C@@H](O)[C@H]2O)n2ncnc(N)c12] |
| Compound 141 | 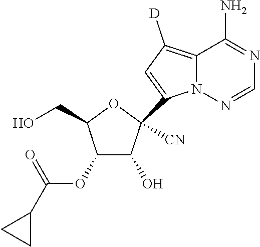 [SMILES: [2H]c1cc([C@]2(C#N)O[C@H](CO)[C@@H](OC(=O)C3CC3)[C@H]2O)n2ncnc(N)c12] |
| Compound 142 | 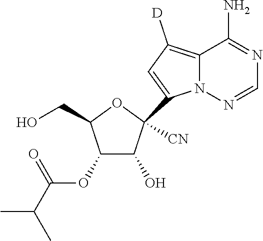 [SMILES: [2H]c1cc([C@]2(C#N)O[C@H](CO)[C@@H](OC(=O)C(C)C)[C@H]2O)n2ncnc(N)c12] |
| Compound 143 | 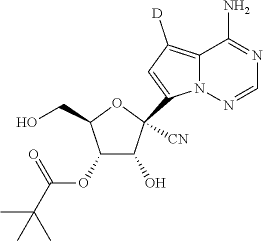 [SMILES: [2H]c1cc([C@]2(C#N)O[C@H](CO)[C@@H](OC(=O)C(C)(C)C)[C@H]2O)n2ncnc(N)c12] |
| Compound 144 | 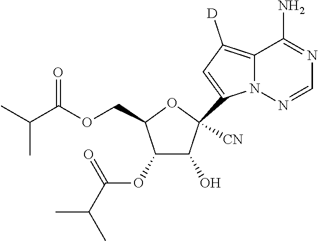 [SMILES: [2H]c1cc([C@]2(C#N)O[C@H](COC(=O)C(C)C)[C@@H](OC(=O)C(C)C)[C@H]2O)n2ncnc(N)c12] |
| Compound 145 | 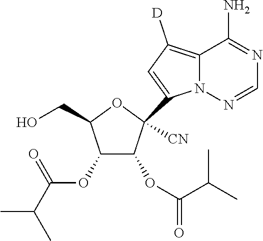 [SMILES: [2H]c1cc([C@]2(C#N)O[C@H](CO)[C@@H](OC(=O)C(C)C)[C@H]2OC(=O)C(C)C)n2ncnc(N)c12] |
| Compound 146 | 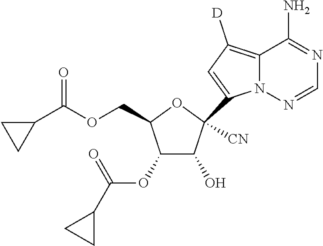 [SMILES: [2H]c1cc([C@]2(C#N)O[C@H](COC(=O)C3CC3)[C@@H](OC(=O)C3CC3)[C@H]2O)n2ncnc(N)c12] |
| Compound 147 | 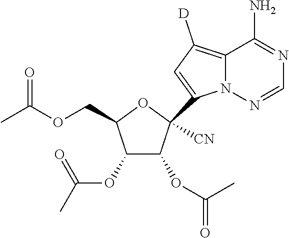 [SMILES: [2H]c1cc([C@]2(C#N)O[C@H](COC(C)=O)[C@@H](OC(C)=O)[C@H]2OC(C)=O)n2ncnc(N)c12] |
| Compound 148 | 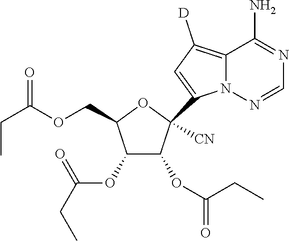 [SMILES: [2H]c1cc([C@]2(C#N)O[C@H](COC(=O)CC)[C@@H](OC(=O)CC)[C@H]2OC(=O)CC)n2ncnc(N)c12] |
| Compound 149 | 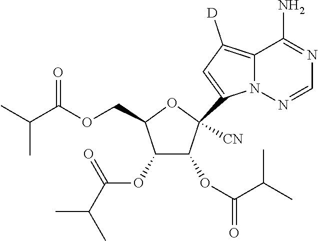 [SMILES: [2H]c1cc([C@]2(C#N)O[C@H](COC(=O)C(C)C)[C@@H](OC(=O)C(C)C)[C@H]2OC(=O)C(C)C)n2ncnc(N)c12] |
| Compound 150 | 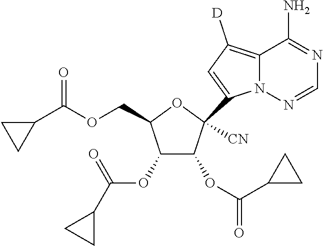 [SMILES: [2H]c1cc([C@]2(C#N)O[C@H](COC(=O)C3CC3)[C@@H](OC(=O)C3CC3)[C@H]2OC(=O)C2CC2)n2ncnc(N)c12] |
| Compound 151 | 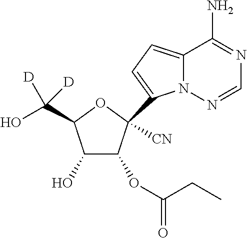 [SMILES: [2H]C([2H])(O)[C@H]1O[C@@](C#N)(c2ccc3c(N)ncnn23)[C@H](OC(=O)CC)[C@@H]1O] |
| Compound 152 | 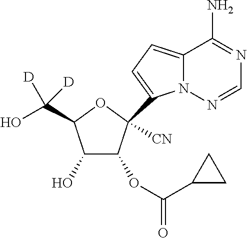 [SMILES: [2H]C([2H])(O)[C@H]1O[C@@](C#N)(c2ccc3c(N)ncnn23)[C@H](OC(=O)C2CC2)[C@@H]1O] |
| Compound 153 | 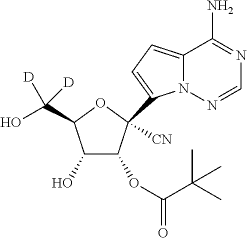 [SMILES: [2H]C([2H])(O)[C@H]1O[C@@](C#N)(c2ccc3c(N)ncnn23)[C@H](OC(=O)C(C)(C)C)[C@@H]1O] |
| Compound 154 | 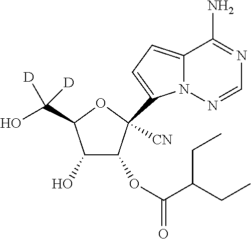 [SMILES: [2H]C([2H])(O)[C@H]1O[C@@](C#N)(c2ccc3c(N)ncnn23)[C@H](OC(=O)C(CC)CC)[C@@H]1O] |
| Compound 155 | 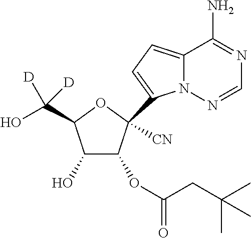 [SMILES: [2H]C([2H])(O)[C@H]1O[C@@](C#N)(c2ccc3c(N)ncnn23)[C@H](OC(=O)CC(C)(C)C)[C@@H]1O] |
| Compound 156 | 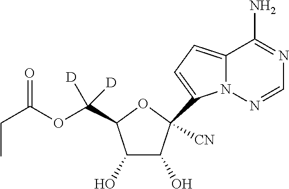 [SMILES: [2H]C([2H])(OC(=O)CC)[C@H]1O[C@@](C#N)(c2ccc3c(N)ncnn23)[C@H](O)[C@@H]1O] |
| Compound 157 | 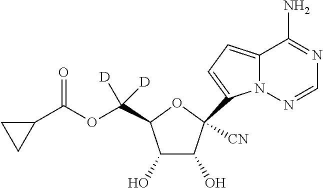 [SMILES: [2H]C([2H])(OC(=O)C1CC1)[C@H]1O[C@@](C#N)(c2ccc3c(N)ncnn23)[C@H](O)[C@@H]1O] |
| Compound 158 | 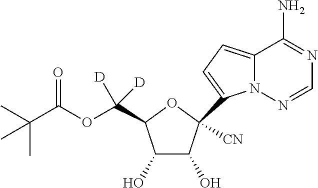 [SMILES: [2H]C([2H])(OC(=O)C(C)(C)C)[C@H]1O[C@@](C#N)(c2ccc3c(N)ncnn23)[C@H](O)[C@@H]1O] |
| Compound 159 | 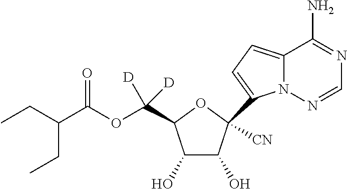 [SMILES: [2H]C([2H])(OC(=O)C(CC)CC)[C@H]1O[C@@](C#N)(c2ccc3c(N)ncnn23)[C@H](O)[C@@H]1O] |
| Compound 160 | 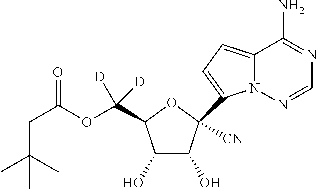 [SMILES: [2H]C([2H])(OC(=O)CC(C)(C)C)[C@H]1O[C@@](C#N)(c2ccc3c(N)ncnn23)[C@H](O)[C@@H]1O] |
| Compound 161 | 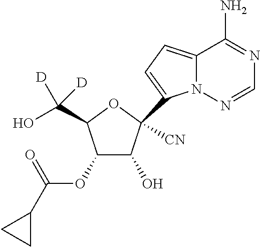 [SMILES: [2H]C([2H])(O)[C@H]1O[C@@](C#N)(c2ccc3c(N)ncnn23)[C@H](O)[C@@H]1OC(=O)C1CC1] |
| Compound 162 | 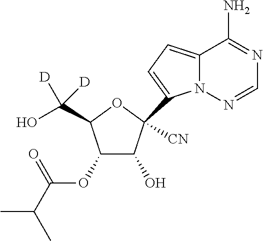 [SMILES: [2H]C([2H])(O)[C@H]1O[C@@](C#N)(c2ccc3c(N)ncnn23)[C@H](O)[C@@H]1OC(=O)C(C)C] |
| Compound 163 | 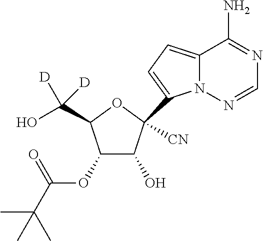 [SMILES: [2H]C([2H])(O)[C@H]1O[C@@](C#N)(c2ccc3c(N)ncnn23)[C@H](O)[C@@H]1OC(=O)C(C)(C)C] |
| Compound 164 | 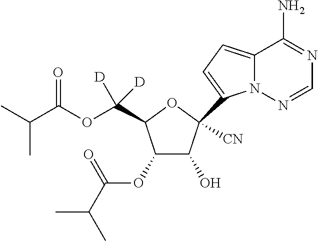 [SMILES: [2H]C([2H])(OC(=O)C(C)C)[C@H]1O[C@@](C#N)(c2ccc3c(N)ncnn23)[C@H](O)[C@@H]1OC(=O)C(C)C] |
| Compound 165 | 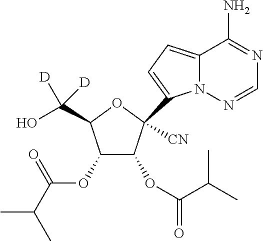 [SMILES: [2H]C([2H])(O)[C@H]1O[C@@](C#N)(c2ccc3c(N)ncnn23)[C@H](OC(=O)C(C)C)[C@@H]1OC(=O)C(C)C] |
| Compound 166 | 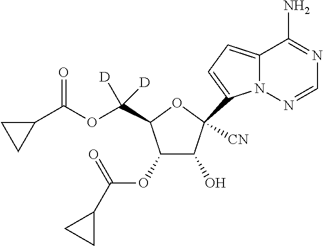 [SMILES: [2H]C([2H])(OC(=O)C1CC1)[C@H]1O[C@@](C#N)(c2ccc3c(N)ncnn23)[C@H](O)[C@@H]1OC(=O)C1CC1] |
| Compound 167 | 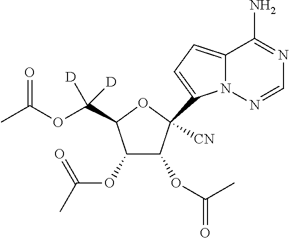 [SMILES: [2H]C([2H])(OC(C)=O)[C@H]1O[C@@](C#N)(c2ccc3c(N)ncnn23)[C@H](OC(C)=O)[C@@H]1OC(C)=O] |
| Compound 168 | 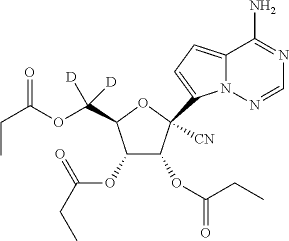 [SMILES: [2H]C([2H])(OC(=O)CC)[C@H]1O[C@@](C#N)(c2ccc3c(N)ncnn23)[C@H](OC(=O)CC)[C@@H]1OC(=O)CC] |
| Compound 169 | 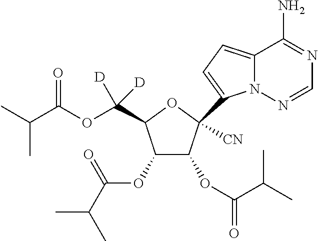 [SMILES: [2H]C([2H])(OC(=O)C(C)C)[C@H]1O[C@@](C#N)(c2ccc3c(N)ncnn23)[C@H](OC(=O)C(C)C)[C@@H]1OC(=O)C(C)C] |
| Compound 170 | 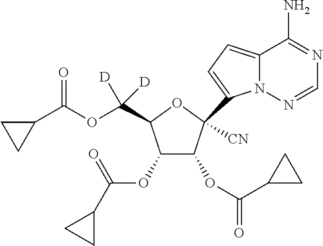 [SMILES: [2H]C([2H])(OC(=O)C1CC1)[C@H]1O[C@@](C#N)(c2ccc3c(N)ncnn23)[C@H](OC(=O)C2CC2)[C@@H]1OC(=O)C1CC1] |
| Compound 171 | 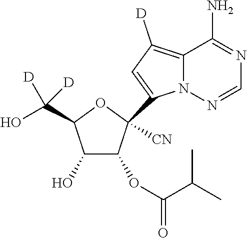 [SMILES: [2H]c1cc([C@]2(C#N)O[C@H](C([2H])([2H])O)[C@@H](O)[C@H]2OC(=O)C(C)C)n2ncnc(N)c12] |
| Compound 172 | 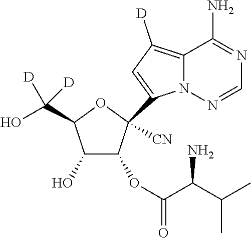 [SMILES: [2H]c1cc([C@]2(C#N)O[C@H](C([2H])([2H])O)[C@@H](O)[C@H]2OC(=O)[C@@H](N)C(C)C)n2ncnc(N)c12] |
| Compound 173 | 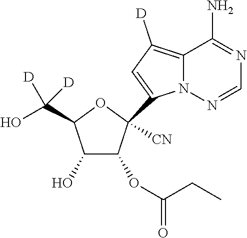 [SMILES: [2H]c1cc([C@]2(C#N)O[C@H](C([2H])([2H])O)[C@@H](O)[C@H]2OC(=O)CC)n2ncnc(N)c12] |
| Compound 174 | 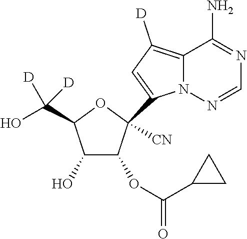 [SMILES: [2H]c1cc([C@]2(C#N)O[C@H](C([2H])([2H])O)[C@@H](O)[C@H]2OC(=O)C2CC2)n2ncnc(N)c12] |
| Compound 175 | 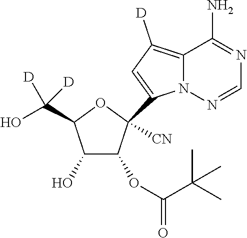 [SMILES: [2H]c1cc([C@]2(C#N)O[C@H](C([2H])([2H])O)[C@@H](O)[C@H]2OC(=O)C(C)(C)C)n2ncnc(N)c12] |
| Compound 176 | 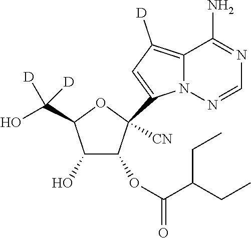 [SMILES: [2H]c1cc([C@]2(C#N)O[C@H](C([2H])([2H])O)[C@@H](O)[C@H]2OC(=O)C(CC)CC)n2ncnc(N)c12] |
| Compound 177 | 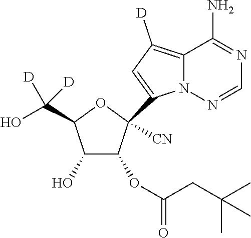 [SMILES: [2H]c1cc([C@]2(C#N)O[C@H](C([2H])([2H])O)[C@@H](O)[C@H]2OC(=O)CC(C)(C)C)n2ncnc(N)c12] |
| Compound 178 | 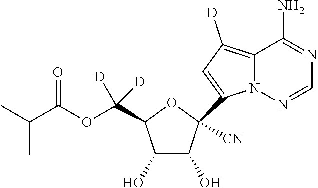 [SMILES: [2H]c1cc([C@]2(C#N)O[C@H](C([2H])([2H])OC(=O)C(C)C)[C@@H](O)[C@H]2O)n2ncnc(N)c12] |
| Compound 179 | 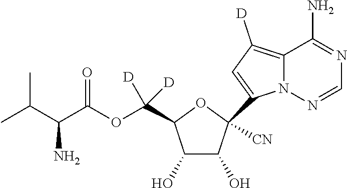 [SMILES: [2H]c1cc([C@]2(C#N)O[C@H](C([2H])([2H])OC(=O)[C@@H](N)C(C)C)[C@@H](O)[C@H]2O)n2ncnc(N)c12] |
| Compound 180 | 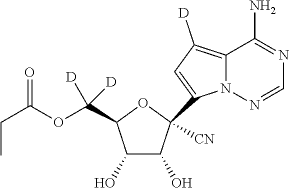 [SMILES: [2H]c1cc([C@]2(C#N)O[C@H](C([2H])([2H])OC(=O)CC)[C@@H](O)[C@H]2O)n2ncnc(N)c12] |
| Compound 181 | 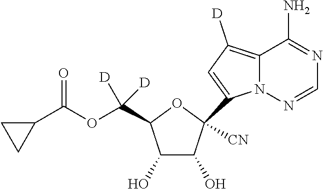 [SMILES: [2H]c1cc([C@]2(C#N)O[C@H](C([2H])([2H])OC(=O)C3CC3)[C@@H](O)[C@H]2O)n2ncnc(N)c12] |
| Compound 182 | 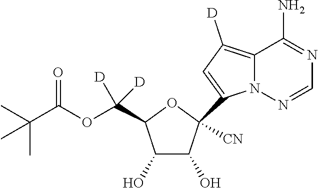 [SMILES: [2H]c1cc([C@]2(C#N)O[C@H](C([2H])([2H])OC(=O)C(C)(C)C)[C@@H](O)[C@H]2O)n2ncnc(N)c12] |
| Compound 183 | 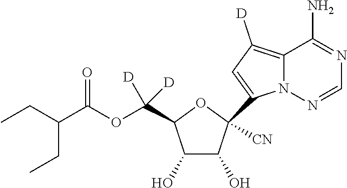 [SMILES: [2H]c1cc([C@]2(C#N)O[C@H](C([2H])([2H])OC(=O)C(CC)CC)[C@@H](O)[C@H]2O)n2ncnc(N)c12] |
| Compound 184 | 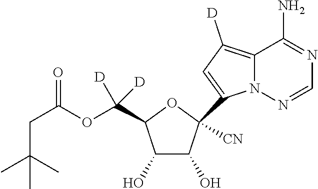 [SMILES: [2H]c1cc([C@]2(C#N)O[C@H](C([2H])([2H])OC(=O)CC(C)(C)C)[C@@H](O)[C@H]2O)n2ncnc(N)c12] |
| Compound 185 | 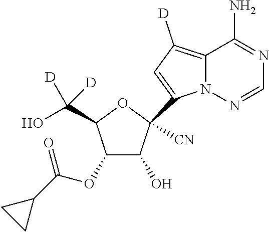 [SMILES: [2H]c1cc([C@]2(C#N)O[C@H](C([2H])([2H])O)[C@@H](OC(=O)C3CC3)[C@H]2O)n2ncnc(N)c12] |
| Compound 186 | 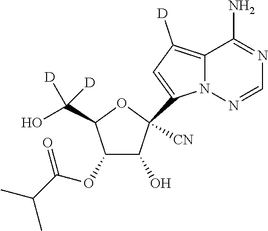 [SMILES: [2H]c1cc([C@]2(C#N)O[C@H](C([2H])([2H])O)[C@@H](OC(=O)C(C)C)[C@H]2O)n2ncnc(N)c12] |
| Compound 187 | 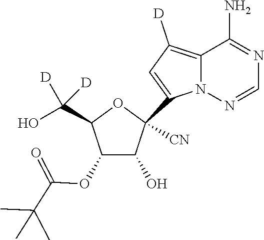 [SMILES: [2H]c1cc([C@]2(C#N)O[C@H](C([2H])([2H])O)[C@@H](OC(=O)C(C)(C)C)[C@H]2O)n2ncnc(N)c12] |
| Compound 188 | 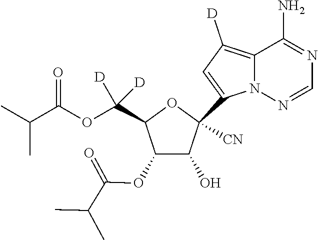 [SMILES: [2H]c1cc([C@]2(C#N)O[C@H](C([2H])([2H])OC(=O)C(C)C)[C@@H](OC(=O)C(C)C)[C@H]2O)n2ncnc(N)c12] |
| Compound 189 | 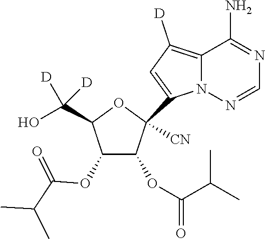 [SMILES: [2H]c1cc([C@]2(C#N)O[C@H](C([2H])([2H])O)[C@@H](OC(=O)C(C)C)[C@H]2OC(=O)C(C)C)n2ncnc(N)c12] |
| Compound 190 | 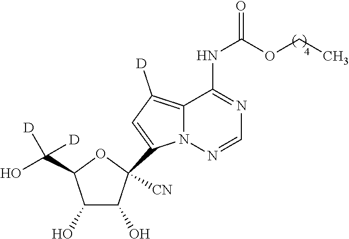 [SMILES: [2H]c1cc([C@]2(C#N)O[C@H](C([2H])([2H])O)[C@@H](O)[C@H]2O)n2ncnc(NC(=O)OCC)c12] |
| Compound 191 | 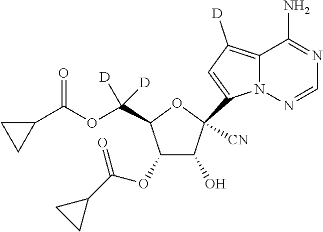 [SMILES: [2H]c1cc([C@]2(C#N)O[C@H](C([2H])([2H])OC(=O)C3CC3)[C@@H](OC(=O)C3CC3)[C@H]2O)n2ncnc(N)c12] |
| Compound 192 | 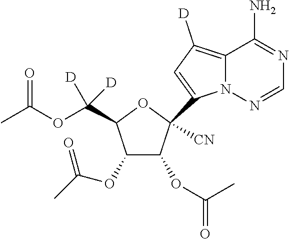 [SMILES: [2H]c1cc([C@]2(C#N)O[C@H](C([2H])([2H])OC(C)=O)[C@@H](OC(C)=O)[C@H]2OC(C)=O)n2ncnc(N)c12] |
| Compound 193 | 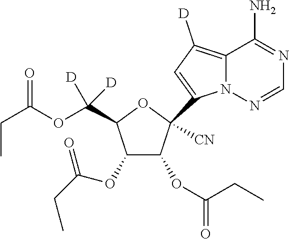 [SMILES: [2H]c1cc([C@]2(C#N)O[C@H](C([2H])([2H])OC(=O)CC)[C@@H](OC(=O)CC)[C@H]2OC(=O)CC)n2ncnc(N)c12] |
| Compound 194 | 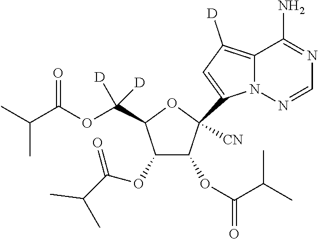 [SMILES: [2H]c1cc([C@]2(C#N)O[C@H](C([2H])([2H])OC(=O)C(C)C)[C@@H](OC(=O)C(C)C)[C@H]2OC(=O)C(C)C)n2ncnc(N)c12] |
| Compound 195 | 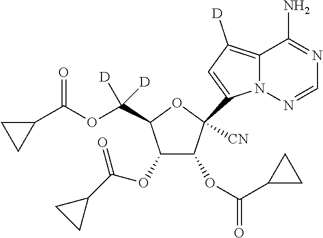 [SMILES: [2H]c1cc([C@]2(C#N)O[C@H](C([2H])([2H])OC(=O)C3CC3)[C@@H](OC(=O)C3CC3)[C@H]2OC(=O)C2CC2)n2ncnc(N)c12] |
| Compound 196 | 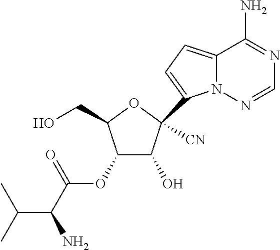 [SMILES: CC(C)[C@H](N)C(=O)O[C@H]1[C@@H](O)[C@](C#N)(c2ccc3c(N)ncnn23)O[C@@H]1CO] |
| Compound 197 | 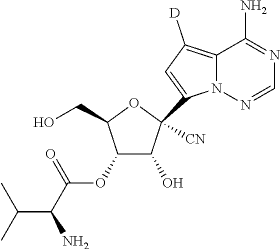 [SMILES: [2H]c1cc([C@]2(C#N)O[C@H](CO)[C@@H](OC(=O)[C@@H](N)C(C)C)[C@H]2O)n2ncnc(N)c12] |
| Compound 198 | 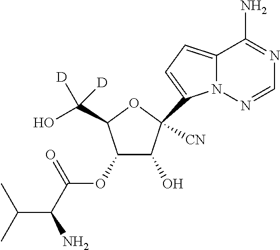 [SMILES: [2H]C([2H])(O)[C@H]1O[C@@](C#N)(c2ccc3c(N)ncnn23)[C@H](O)[C@@H]1OC(=O)[C@@H](N)C(C)C] |
| Compound 199 | 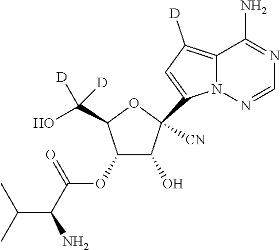 [SMILES: [2H]c1cc([C@]2(C#N)O[C@H](C([2H])([2H])O)[C@@H](OC(=O)[C@@H](N)C(C)C)[C@H]2O)n2ncnc(N)c12] |
| Compound 200 | 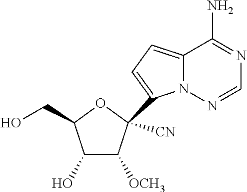 [SMILES: CO[C@@H]1[C@H](O)[C@@H](CO)O[C@@]1(C#N)c1ccc2c(N)ncnn12] |

In some embodiments, the deuterated compound of Formula A, the prodrug of the compound of Formula A, or the prodrug of the deuterated compound of Formula A is a compound of Table 3A, or a pharmaceutically acceptable salt thereof.

TABLE 3A

| | |
|---|---|
| Compound 201 | 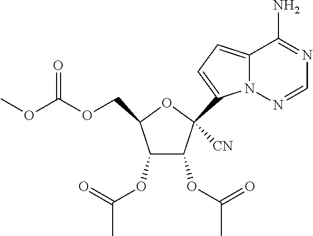 [SMILES: COC(=O)OC[C@H]1O[C@@](C#N)(c2ccc3c(N)ncnn23)[C@H](OC(C)=O)[C@@H]1OC(C)=O] |
| Compound 202 | 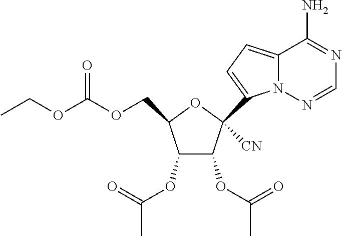 [SMILES: CCOC(=O)OC[C@H]1O[C@@](C#N)(c2ccc3c(N)ncnn23)[C@H](OC(C)=O)[C@@H]1OC(C)=O] |
| Compound 203 | 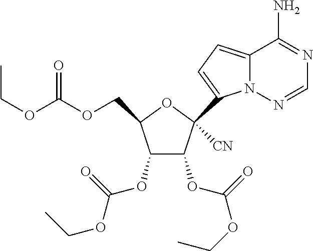 [SMILES: CCOC(=O)OC[C@H]1O[C@@](C#N)(c2ccc3c(N)ncnn23)[C@H](OC(=O)OCC)[C@@H]1OC(=O)OCC] |
| Compound 204 | 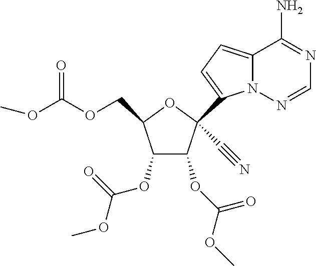 [SMILES: COC(=O)OC[C@H]1O[C@@](C#N)(c2ccc3c(N)ncnn23)[C@H](OC(=O)OC)[C@@H]1OC(=O)OC] |

Compounds 2-33 and their syntheses are disclosed in WO/2022/047065 and incorporated herein by reference in its entireties. Compounds 34-54, 59, 60, 62, 64, 68, 70, 73, and 201-204 and their syntheses are disclosed in PCT/US2023/014299 and incorporated herein by reference in its entireties. Compounds 55-58, 61, 63, 65-67, 69, 71, 72, and 74-78 are disclosed in PCT/US2023/014299 and incorporated herein by reference in its entireties. Compounds 79, 80, and 82-88 and their syntheses are disclosed in PCT/US2023/014293 and incorporated herein by reference in its entireties. Compounds 81 and 89-91 are disclosed in PCT/US2023/014293 and incorporated herein by reference in its entireties. Compounds 92-200 are disclosed in PCT/US2023/024473 and incorporated herein by reference in its entireties.

In some embodiments, the compound of Formula I is

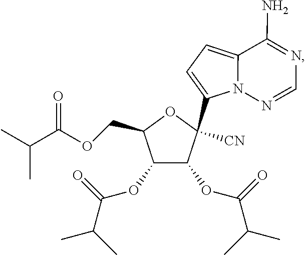 [SMILES: CC(C)C(=O)OC[C@H]1O[C@@](C#N)(c2ccc3c(N)ncnn23)[C@H](OC(=O)C(C)C)[C@@H]1OC(=O)C(C)C]

or a pharmaceutically acceptable salt thereof.

In some embodiments, the deuterated compound of Formula I is

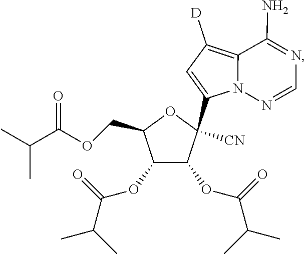 [SMILES: [2H]c1cc([C@]2(C#N)O[C@H](COC(=O)C(C)C)[C@@H](OC(=O)C(C)C)[C@H]2OC(=O)C(C)C)n2ncnc(N)c12]

or a pharmaceutically acceptable salt thereof.

In some embodiments, the compound of Formula I is

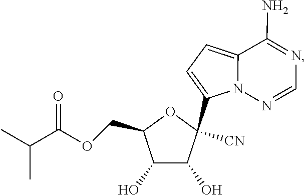 [SMILES: CC(C)C(=O)OC[C@H]1O[C@@](C#N)(c2ccc3c(N)ncnn23)[C@H](O)[C@@H]1O]

or a pharmaceutically acceptable salt thereof.

Compounds for Use in Treating Viral Infections

The application provides a compound which is:

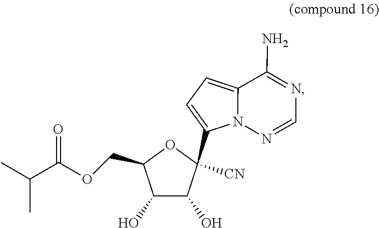 [SMILES: CC(C)C(=O)OC[C@H]1O[C@@](C#N)(c2ccc3c(N)ncnn23)[C@H](O)[C@@H]1O]

a deuterated compound thereof, or a pharmaceutically acceptable salt thereof, for use in a method of treating a viral infection in a patient in need thereof, wherein the method comprises:
administering compound 16, a deuterated compound thereof, or a pharmaceutically acceptable salt thereof at a dose of 100 mg/dose to 1600 mg/dose if the patient's eGFR is at least 60 mL/min/1.73 m²; and
administering compound 16, a deuterated compound thereof, or a pharmaceutically acceptable salt thereof at a dose of 50 mg/dose to 900 mg/dose if the patient's eGFR is less than 60 mL/min/1.73 m².

In some embodiments, the method further comprises the step of determining an eGFR of the patient.

In some embodiments, if the patient has an eGFR which is at least 60 mL/min/1.73 m², the compound is administered twice daily and if the patient has an eGFR which is less than 60 mL/min/1.73 m², the compound is administered once daily.

In some embodiments, the compound is administered at a dose of:
100 mg/dose to 900 mg/dose if the patient's eGFR is at least 60 mL/min/1.73 m²; and
50 mg/dose to 700 mg/dose if the patient's eGFR is less than 60 mL/min/1.73 m².

In some embodiments, the compound is administered at a dose of:
100 mg/dose to 700 mg/dose if the patient's eGFR is at least 60 mL/min/1.73 m²; and
50 mg/dose to 500 mg/dose if the patient's eGFR is less than 60 mL/min/1.73 m².

In some embodiments, the compound is administered at a dose of:
100 mg/dose to 500 mg/dose if the patient's eGFR is at least 60 mL/min/1.73 m²; and
50 mg/dose to 400 mg/dose if the patient's eGFR is less than 60 mL/min/1.73 m².

In some embodiments, the compound is administered at a dose of:
100 mg/dose to 900 mg/dose twice daily if the patient's eGFR is at least 60 mL/min/1.73 m²; and
50 mg/dose to 700 mg/dose once daily if the patient's eGFR is less than 60 mL/min/1.73 m².

In some embodiments, the compound is administered at a dose of:
100 mg/dose to 700 mg/dose twice daily if the patient's eGFR is at least 60 mL/min/1.73 m²; and
50 mg/dose to 500 mg/dose once daily if the patient's eGFR is less than 60 mL/min/1.73 m².

In some embodiments, the compound is administered at a dose of:
100 mg/dose to 500 mg/dose twice daily if the patient's eGFR is at least 60 mL/min/1.73 m²; and
50 mg/dose to 400 mg/dose once daily if the patient's eGFR is less than 60 mL/min/1.73 m².

In some embodiments, the compound is administered at a dose of:
200 mg/dose to 500 mg/dose twice daily if the patient's eGFR is at least 60 mL/min/1.73 m²; and
100 mg/dose to 400 mg/dose once daily if the patient's eGFR is 30-59 mL/min/1.73 m²; and
50 mg/dose to 400 mg/dose once daily if the patient's eGFR is 15-29 mL/min/1.73 m².

In some embodiments, the compound is administered at a dose of:
200 mg/dose to 400 mg/dose twice daily if the patient's eGFR is at least 60 mL/min/1.73 m²;
200 mg/dose to 400 mg/dose once daily if the patient's eGFR is 30-59 mL/min/1.73 m²; and
100 mg/dose to 400 mg/dose once daily if the patient's eGFR is 15-29 mL/min/1.73 m².

In some embodiments, the compound is administered at a dose of:
200 mg/dose to 400 mg/dose once daily if the patient's eGFR is at least 60 mL/min/1.73 m²;
200 mg/dose to 400 mg/dose once daily if the patient's eGFR is 30-59 mL/min/1.73 m²; and
200 mg/dose to 400 mg/dose once daily on a first day of administration, and 100 mg/dose to 200 mg/dose once daily on each day of administration after the first day if the patient's eGFR is 15-29 mL/min/1.73 m².

In some embodiments, the compound is administered at a dose of:
350 mg/dose twice daily if the patient's eGFR is at least 60 mL/min/1.73 m²;
350 mg/dose once daily if the patient's eGFR is 30-59 mL/min/1.73 m²; and
350 mg/dose once daily on a first day of administration, and 150 mg/dose once daily on each day of administration after the first day if the patient's eGFR is 15-29 mL/min/1.73 m².

In some embodiments, the compound is a deuterated compound of compound 16, which is

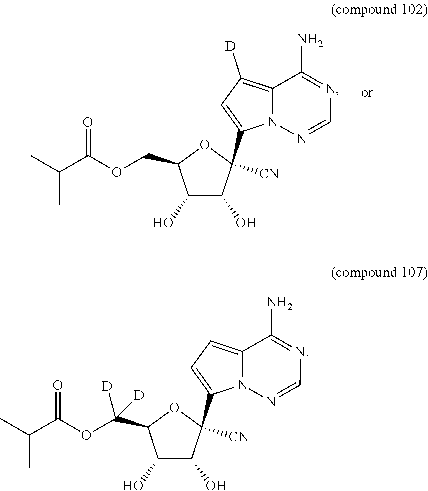 [SMILES: [2H]c1cc([C@]2(C#N)O[C@H](COC(=O)C(C)C)[C@@H](O)[C@H]2O)n2ncnc(N)c12 ; [2H]C([2H])(OC(=O)C(C)C)[C@H]1O[C@@](C#N)(c2ccc3c(N)ncnn23)[C@H](O)[C@@H]1O]

### Pharmaceutical Formulations

The compounds described herein may be formulated with conventional carriers and excipients. For example, tablets will contain excipients, glidants, fillers, binders and the like. Aqueous formulations are prepared in sterile form, and when intended for delivery by other than oral administration generally will be isotonic. All formulations may optionally comprise excipients such as those set forth in the “Handbook of Pharmaceutical Excipients” (1986). Pharmaceutically acceptable excipients include ascorbic acid and other antioxidants, chelating agents such as EDTA, carbohydrates such as dextran, hydroxyalkylcellulose, hydroxyalkylmethylcellulose, stearic acid and the like. In some embodiments, the formulations comprise one or more pharmaceutically acceptable excipients. The pH of the formulations ranges from about 3 to about 11, but is ordinarily about 7 to 10. In some embodiments, the pH of the formulations ranges from about 2 to about 5, but is ordinarily about 3 to 4.

While it is possible for the compounds of the disclosure (“the active ingredients”) to be administered alone it may be preferable to present them as pharmaceutical formulations. The formulations, both for veterinary and for human use, of the invention comprise at least one active ingredient, as above defined, together with one or more acceptable carriers therefor and optionally other therapeutic ingredients, particularly those additional therapeutic ingredients as discussed herein. The carrier(s) must be “acceptable” in the sense of being compatible with the other ingredients of the formulation and physiologically innocuous to the recipient thereof.

The formulations include those suitable for the foregoing administration routes. The formulations may conveniently be presented in unit dosage form and may be prepared by any appropriate method known in the art of pharmacy. Techniques and formulations generally are found in Remington's Pharmaceutical Sciences (Mack Publishing Co., Easton, PA). Such methods include the step of bringing into association the active ingredient with the carrier which constitutes one or more accessory ingredients. In general, the formulations are prepared by uniformly and intimately bringing into association the active ingredient with liquid carriers or finely divided solid carriers or both, and then, if necessary, shaping the product.

In some embodiments, the pharmaceutical formulation is for subcutaneous, intramuscular, intravenous, oral, or inhalation administration.

In some embodiments, a compound described herein, e.g., a compound of Formula A, a deuterated compound of Formula A, a prodrug of the compound of Formula A, a prodrug of the deuterated compound of Formula A, a compound of Formula I, a deuterated compound of Formula I, or a pharmaceutically acceptable salt thereof, described herein have optimized/improved pharmacokinetic properties and are amenable to oral administration. For example, the compounds of Formula I have improved bioavailability and can therefore be administered by oral administration.

In some embodiments, the formulations of the present invention are suitable for oral administration may be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste.

In some embodiments, the formulation is a tablet and the tablet is made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

For infections of the eye or other external tissues, e.g., mouth and skin, the formulations are applied as a topical ointment or cream containing the active ingredient(s) in an amount of, for example, 0.075 to 20% w/w (including active ingredient(s) in a range between 0.1% and 20% in increments of 0.1% w/w such as 0.6% w/w, 0.7% w/w, etc.), preferably 0.2 to 15% w/w and most preferably 0.5 to 10% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include, for example, at least 30% w/w of a polyhydric alcohol, i.e., an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulphoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

Emulgents and emulsion stabilizers suitable for use in the formulation of the invention include Tween® 60, Span® 80, cetostearyl alcohol, benzyl alcohol, myristyl alcohol, glyceryl mono-stearate and sodium lauryl sulfate. Further emulgents and emulsion stabilizers suitable for use in the formulation of the invention include Tween® 80.

The choice of suitable oils or fats for the formulation is based on achieving the desired cosmetic properties. The cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils are used.

Pharmaceutical formulations according to the present invention comprise a compound according to the invention together with one or more pharmaceutically acceptable carriers or excipients and optionally other therapeutic agents. Pharmaceutical formulations containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

Aqueous suspensions of the invention contain the active materials in admixture with excipients suitable for the manufacture of aqueous suspensions. Such excipients include a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally-occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin. Further non-limiting examples of suspending agents include Cyclodextrin. In some examples, the suspending agent is Sulfobutyl ether beta-cyclodextrin (SEB-beta-CD), for example Captisol®.

Oil suspensions may be formulated by suspending the active ingredient in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Dispersible powders and granules of the invention suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the invention may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally-occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

The pharmaceutical compositions of the invention may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose, any bland fixed oil may be employed including synthetic mono- or diglycerides. In addition, fatty acids such as oleic acid may likewise be used in the preparation of injectables. Among the acceptable vehicles and solvents that may be employed are water, Ringer's solution isotonic sodium chloride solution, and hypertonic sodium chloride solution.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 mg of the active ingredient per milliliter of solution in order that infusion of a suitable volume at a rate of about 30 mL/hr can occur.

Formulations suitable for topical administration to the eye also include eye drops wherein the active ingredient is dissolved or suspended in a suitable carrier, especially an aqueous solvent for the active ingredient. The active ingredient is preferably present in such formulations in a concentration of 0.5 to 20%, advantageously 0.5 to 10%, and particularly about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

In some embodiments, the compounds described herein are administered by inhalation. In some embodiments, formulations suitable for intrapulmonary or nasal administration have a particle size for example in the range of 0.1 to 500 microns, such as 0.5, 1, 30, 35 etc., which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents. In some embodiments, the compounds used herein are formulated and dosed as dry powder. In some embodiments, the compounds used herein are formulated and dosed as a nebulized formulation. In some embodiments, the compounds used herein are formulated for delivery by a face mask. In some embodiments, the compounds used herein are formulated for delivery by a face tent.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations are presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

It should be understood that in addition to the ingredients particularly mentioned above the formulations of this invention may include other agents conventional in the art having regard to the type of formulation in question, for example those suitable for oral administration may include flavoring agents.

The invention further provides veterinary compositions comprising at least one active ingredient as above defined together with a veterinary carrier therefor.

Veterinary carriers are materials useful for the purpose of administering the composition and may be solid, liquid or gaseous materials which are otherwise inert or acceptable in the veterinary art and are compatible with the active ingredient. These veterinary compositions may be administered orally, parenterally or by any other desired route.

Compounds described herein are used to provide controlled release pharmaceutical formulations containing as active ingredient one or more compounds described herein (“controlled release formulations”) in which the release of the active ingredient are controlled and regulated to allow less frequency dosing or to improve the pharmacokinetic or toxicity profile of a given active ingredient.

Also provided herein are kits that include a compound described herein. In some embodiments the kits described herein may comprise a label and/or instructions for use of the compound in the treatment of a disease or condition in a non-pregnant patient in need thereof. In some embodiments, the disease or condition is viral infection.

In some embodiments, the kit may also comprise one or more additional therapeutic agents and/or instructions for use of additional therapeutic agents in combination with a compound described herein in the treatment of the disease or condition in a subject (e.g., human) in need thereof.

In some embodiments, the kits provided herein comprises individual dose units of a compound described herein. Examples of individual dosage units may include pills, tablets, capsules, prefilled syringes or syringe cartridges, IV bags, inhalers, nebulizers etc., each comprising a therapeutically effective amount of a compound described herein. In some embodiments, the kit may contain a single dosage unit and in others, multiple dosage units are present, such as the number of dosage units required for a specified regimen or period.

One or more compounds described herein are administered by any route appropriate to the condition to be treated. Suitable routes include oral, rectal, inhalation, pulmonary, topical (including buccal and sublingual), vaginal and parenteral (including subcutaneous, intramuscular, intravenous, intradermal, intrathecal and epidural), and the like. In some embodiments, the compounds described herein are administered by inhalation or intravenously. In some embodiments, the compounds described herein are administered orally. It will be appreciated that the preferred route may vary with for example the condition of the recipient.

In the methods described herein for the treatment of a viral infection, the compounds described herein can be administered at any time to a human who may come into contact with the virus or is already suffering from the viral infection. In some embodiments, the compounds described herein can be administered prophylactically to humans coming into contact with humans suffering from the viral infection or at risk of coming into contact with humans suffering from the viral infection, e.g., healthcare providers. In some embodiments, administration of the compounds described herein can be to humans testing positive for the viral infection but not yet showing symptoms of the viral infection. In some embodiments, administration of the compounds described herein can be to humans upon commencement of symptoms of the viral infection.

In some embodiments, the methods described herein comprise event driven administration of a compound described herein, e.g., the compound of Formula A, the deuterated compound of Formula A, the prodrug of the compound of Formula A, the prodrug of the deuterated compound of Formula A, the compound of Formula I, the deuterated compound of Formula I, or the pharmaceutically acceptable salt thereof, to the subject.

As used herein, the terms “event driven” or “event driven administration” refer to administration of a compound described herein (e.g., a compound of Formula A, a deuterated compound of Formula A, a prodrug of the compound of Formula A, a prodrug of the deuterated compound of Formula A, a compound of Formula I, a deuterated compound of Formula I, or a pharmaceutically acceptable salt thereof) (1) prior to an event (e.g., 2 hours, 1 day, 2 days, 5 day, or 7 or more days prior to the event) that would expose the individual to the virus (or that would otherwise increase the individual's risk of acquiring the viral infection); and/or (2) during an event (or more than one recurring event) that would expose the individual to the virus (or that would otherwise increase the individual's risk of acquiring the viral infection); and/or (3) after an event (or after the final event in a series of recurring events) that would expose the individual to the virus (or that would otherwise increase the individual's risk of acquiring the viral infection). In some embodiments, the event driven administration is performed pre-exposure of the subject to the virus. In some embodiments, the event driven administration is performed post-exposure of the subject to the virus. In some embodiments, the event driven administration is performed pre-exposure of the subject to the virus and post-exposure of the subject to the virus.

In certain embodiments, the methods described herein involve administration prior to and/or after an event that would expose the individual to the virus or that would otherwise increase the individual's risk of acquiring the viral infection, e.g., as pre-exposure prophylaxis (PrEP) and/or as post-exposure prophylaxis (PEP). In some embodiments, the methods described herein comprise pre-exposure prophylaxis (PrEP). In some embodiments, methods described herein comprise post-exposure prophylaxis (PEP).

In some embodiments, a compound described herein is administered before exposure of the subject to the virus.

In some embodiments, a compound described herein is administered before and after exposure of the subject to the virus.

In some embodiments, a compound described herein is administered after exposure of the subject to the virus.

An example of event driven dosing regimen includes administration of a compound described herein within 24 to 2 hours prior to the virus, followed by administration of a compound described herein every 24 hours during the period of exposure, followed by a further administration of a compound described herein after the last exposure, and one last administration of a compound described herein 24 hours later.

A further example of an event driven dosing regimen includes administration of a compound described herein within 24 hours before the viral exposure, then daily administration during the period of exposure, followed by a last administration approximately 24 hours later after the last exposure (which may be an increased dose, such as a double dose).

The specific dose level of a compound described herein for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

The daily dosage may also be described as a total amount of a compound described herein administered per dose or per day. For example, daily dosage of a compound of Formula A, a deuterated compound of Formula A, a prodrug of the compound of Formula A, a prodrug of the deuterated compound of Formula A, a compound of Formula I, a deuterated compound of Formula I, or a pharmaceutically acceptable salt thereof, may be between about 1 mg and 4,000 mg, between about 2,000 to 4,000 mg/day, between about 1 to 2,000 mg/day, between about 1 to 1,000 mg/day, between about 10 to 500 mg/day, between about 20 to 500 mg/day, between about 50 to 300 mg/day, between about 75 to 200 mg/day, or between about 15 to 150 mg/day.

The dosage or dosing frequency of a compound described herein may be adjusted over the course of the treatment, based on the judgment of the administering physician.

The compounds of the present disclosure may be administered to an individual (e.g., a human) in a therapeutically effective amount. In some embodiments, the compound is administered once daily. In some embodiments, the compound is administered twice daily.

The compounds described herein can be administered by any useful route and means, such as by oral or parenteral (e.g., intravenous) administration. Therapeutically effective amounts of the compound may include from about 0.00001 mg/kg body weight per day to about 10 mg/kg body weight per day, such as from about 0.0001 mg/kg body weight per day to about 10 mg/kg body weight per day, or such as from about 0.001 mg/kg body weight per day to about 1 mg/kg body weight per day, or such as from about 0.01 mg/kg body weight per day to about 1 mg/kg body weight per day, or such as from about 0.05 mg/kg body weight per day to about 0.5 mg/kg body weight per day.

A compound described herein may be combined with one or more additional therapeutic agents in any dosage amount of the compound described herein (e.g., from 1 mg to 1000 mg of compound). Therapeutically effective amounts may include from about 0.1 mg per dose to about 1000 mg per dose, such as from about 50 mg per dose to about 500 mg per dose, or such as from about 100 mg per dose to about 400 mg per dose, or such as from about 150 mg per dose to about 350 mg per dose, or such as from about 200 mg per dose to about 300 mg per dose, or such as from about 0.01 mg per dose to about 1000 mg per dose, or such as from about 0.01 mg per dose to about 100 mg per dose, or such as from about 0.1 mg per dose to about 100 mg per dose, or such as from about 1 mg per dose to about 100 mg per dose, or such as from about 1 mg per dose to about 10 mg per dose, or such as from about 1 mg per dose to about 1000 mg per dose. Other therapeutically effective amounts of a compound of Formula A, a deuterated compound of Formula A, a prodrug of the compound of Formula A, a prodrug of the deuterated compound of Formula A, a compound of Formula I, a deuterated compound of Formula I, or a pharmaceutically acceptable salt thereof, are about 1 mg per dose, or about 2, 3, 4, 5, 6, 7, 8, 9, 10, 15, 20, 25, 30, 35, 40, 45, 50, 55, 60, 65, 70, 75, 80, 85, 90, 95, or about 100 mg per dose. Other therapeutically effective amounts of a compound described herein are about 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or about 1000 mg per dose.

In some embodiments, the methods described herein comprise administering to the subject an initial daily dose of about 1 to 500 mg of a compound described herein and increasing the dose by increments until clinical efficacy is achieved. Increments of about 5, 10, 25, 50, or 100 mg can be used to increase the dose. The dosage can be increased daily, every other day, twice per week, once per week, once every two weeks, once every three weeks, or once a month.

In some embodiments, the methods described herein comprise administering to the subject a loading dose of a compound described herein, followed by administering a maintenance dose of the compound on each subsequent day (e.g., once daily on each subsequent day). The maintenance dose of the one or more compounds may be administered for as long as required, for example for up to 5 days, up to 7 days, up to 10 days, up to 15 days, up to 20 days, up to 25 days, up to a month or longer. In some embodiments, the once daily maintenance dose is administered for about 6-12 days, for example for about example 8-10 days. In some embodiments, the once daily maintenance dose is administered for about 4 days. In some embodiments, the once daily maintenance dose is administered for about 5 days. In some embodiments, the once daily maintenance dose is administered for about 9 days. In some embodiments, the once daily maintenance dose is administered for about 10 days. The loading dose may be equal to, less than, or greater than the maintenance dose. In some embodiments, the loading dose is greater than the maintenance dose.

When administered orally, the total daily dosage for a human subject may be between about 1-4,000 mg/day, between about 1-3,000 mg/day, between 1-2,000 mg/day, about 1-1,000 mg/day, between about 10-500 mg/day, between about 50-300 mg/day, between about 75-200 mg/day, or between about 100-150 mg/day. In some embodiments, the total daily dosage for a human subject may be about 100, 200, 300, 400, 500, 600, 700, 800, 900, 1000, 1100, 1200, 1300, 1400, 1500, 1,600, 1700, 1800, 1900, 2000, 2100, 2200, 2300, 2400, 2500, 2600, 2700, 2800, 2900, or 3000 mg/day administered in a single dose. In some embodiments, the total daily dosage for a human subject may be about 200, 300, 400, 500, 600, 700, or 800 mg/day administered in a single dose. In some embodiments, the total daily dosage for a human subject may be about 300, 400, 500, or 600 mg/day administered in a single dose. In some embodiments, the total daily dosage for a human subject may be about 100, 200, 300, 400, 500, 600, 700, 800, 900, 1000, 1100, 1200, 1300, 1400, 1500, 1,600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, or 4000 mg/day. In some embodiments, the total daily dosage for a human subject may be about 100-200, 100-300, 100-400, 100-500, 100-600, 100-700, 100-800, 100-900, 100-1000, 500-1100, 500-1200, 500-1300, 500-1400, 500-1500, 500-1,600, 500-1700, 500-1800, 500-1900, 500-2000, 1500-2100, 1500-2200, 1500-2300, 1500-2400, 1500-2500, 2000-2600, 2000-2700, 2000-2800, 2000-2900, 2000-3000, 2500-3100, 2500-3200, 2500-3300, 2500-3400, 2500-3500, 3000-3600, 3000-3700, 3000-3800, 3000-3900, or 3000-4000 mg/day.

In some embodiments, the total daily dosage for a human subject may be about 100 mg/day administered in a single dose. In some embodiments, the total daily dosage for a human subject may be about 150 mg/day administered in a single dose. In some embodiments, the total daily dosage for a human subject may be about 200 mg/day administered in a single dose. In some embodiments, the total daily dosage for a human subject may be about 250 mg/day administered in a single dose. In some embodiments, the total daily dosage for a human subject may be about 300 mg/day administered in a single dose. In some embodiments, the total daily dosage for a human subject may be about 350 mg/day administered in a single dose. In some embodiments, the total daily dosage for a human subject may be about 400 mg/day administered in a single dose. In some embodiments, the total daily dosage for a human subject may be about 450 mg/day administered in a single dose. In some embodiments, the total daily dosage for a human subject may be about 500 mg/day administered in a single dose. In some embodiments, the total daily dosage for a human subject may be about 550 mg/day administered in a single dose. In some embodiments, the total daily dosage for a human subject may be about 600 mg/day administered in a single dose. In some embodiments, the total daily dosage for a human subject may be about 650 mg/day administered in a single dose. In some embodiments, the total daily dosage for a human subject may be about 700 mg/day administered in a single dose. In some embodiments, the total daily dosage for a human subject may be about 750 mg/day administered in a single dose. In some embodiments, the total daily dosage for a human subject may be about 800 mg/day administered in a single dose. In some embodiments, the total daily dosage for a human subject may be about 850 mg/day administered in a single dose. In some embodiments, the total daily dosage for a human subject may be about 900 mg/day administered in a single dose. In some embodiments, the total daily dosage for a human subject may be about 950 mg/day administered in a single dose. In some embodiments, the total daily dosage for a human subject may be about 1000 mg/day administered in a single dose. In some embodiments, the total daily dosage for a human subject may be about 1500 mg/day administered in a single dose. In some embodiments, the total daily dosage for a human subject may be about 2000 mg/day administered in a single dose. In some embodiments, the total daily dosage for a human subject may be about 2500 mg/day administered in a single dose. In some embodiments, the total daily dosage for a human subject may be about 3000 mg/day administered in a single dose. In some embodiments, the total daily dosage for a human subject may be about 4000 mg/day administered in a single dose.

In some embodiments, the total daily dosage for a human subject may be about 175 mg/day administered in a single dose.

A single dose can be administered hourly, daily, weekly, or monthly. For example, a single dose can be administered once every 1 hour, 2, 3, 4, 6, 8, 12, 16 or once every 24 hours. A single dose can also be administered once every 1 day, 2, 3, 4, 5, 6, or once every 7 days. A single dose can also be administered once every 1 week, 2, 3, or once every 4 weeks. In certain embodiments, a single dose can be administered once every week. A single dose can also be administered once every month. In some embodiments, a compound described herein is administered once daily in a method described herein. In some embodiments, a compound described herein is administered twice daily in a method described herein. In some embodiments, a compound described herein is administered three times daily in a method described herein.

In some embodiments, a compound described herein is administered once daily in the total daily dose of 100-4000 mg/day. In some embodiments, a compound described herein is administered twice daily in the total daily dose of 100-4000 mg/day. In some embodiments, a compound described herein is administered three times daily in the total daily dose of 100-4000 mg/day.

The frequency of dosage of a compound described herein will be determined by the needs of the individual patient and can be, for example, once per day or twice, or more times, per day. Administration of the compound continues for as long as necessary to treat the viral infection. For example, a compound can be administered to a human being infected with the virus for a period of from 20 days to 180 days or, for example, for a period of from 20 days to 90 days or, for example, for a period of from 30 days to 60 days.

Administration can be intermittent, with a period of several or more days during which a patient receives a daily dose of a compound described herein followed by a period of several or more days during which a patient does not receive a daily dose of the compound. For example, a patient can receive a dose of the compound every other day, or three times per week. Again by way of example, a patient can receive a dose of the compound each day for a period of from 1 to 14 days, followed by a period of 7 to 21 days during which the patient does not receive a dose of the compound, followed by a subsequent period (e.g., from 1 to 14 days) during which the patient again receives a daily dose of the compound. Alternating periods of administration of the compound, followed by non-administration of the compound, can be repeated as clinically required to treat the patient.

The compounds of the present disclosure or the pharmaceutical compositions thereof may be administered once, twice, three, or four times daily, using any suitable mode described above. Also, administration or treatment with the compounds may be continued for a number of days; for example, commonly treatment would continue for at least 7 days, 14 days, or 28 days, for one cycle of treatment. Treatment cycles are well known in cancer chemotherapy, and are frequently alternated with resting periods of about 1 to 28 days, commonly about 7 days or about 14 days, between cycles. The treatment cycles, in other embodiments, may also be continuous.

In some embodiments, the compound is administered for 1 to 30 consecutive days, for example, 1 to 28 consecutive days, 1 to 21 consecutive days, 1 to 14 consecutive days, 1 to 7 consecutive days, 1 to 5 consecutive days, 3 to 30 consecutive days, 3 to 28 consecutive days, 3 to 21 consecutive days, 3 to 14 consecutive days, 3 to 7 consecutive days, 5 to 30 consecutive days, 5 to 28 consecutive days, 5 to 21 consecutive days, 5 to 14 consecutive days, or 5 to 7 consecutive days. In some embodiments, the compound is administered once daily or twice daily. In some embodiments, the compound is administered once daily. In some embodiments, the compound is administered twice daily.

In some embodiments, the compound is administered once daily for 2 consecutive days, 3 consecutive days, 4 consecutive days, 5 consecutive days, 6 consecutive days, 7 consecutive days, 8 consecutive days, 9 consecutive days, 10 consecutive days, 11 consecutive days, 12 consecutive days, 13 consecutive days, or 14 consecutive days. In some embodiments, the compound is administered once daily for 3 consecutive days. In some embodiments, the compound is administered once daily for 5 consecutive days. In some embodiments, the compound is administered twice daily for 2 consecutive days, 3 consecutive days, 4 consecutive days, 5 consecutive days, 6 consecutive days, 7 consecutive days, 8 consecutive days, 9 consecutive days, 10 consecutive days, 11 consecutive days, 12 consecutive days, 13 consecutive days, or 14 consecutive days. In some embodiments, the compound is administered twice daily for 3 consecutive days. In some embodiments, the compound is administered twice daily for 5 consecutive days.

In some embodiments, a compound described herein is administered to the human via oral, intramuscular, intravenous, subcutaneous, or inhalation administration. In some embodiments, the compound is administered orally.

Also provided here are the uses of the compounds described herein for use in treating or preventing a viral infection in a subject in need thereof. For example, provided herein are uses of the compounds described herein for use in treating a viral infection in a subject in need thereof.

In some embodiments, the viral infection is a paramyxoviridae virus infection. As such, in some embodiments, the present disclosure provides methods for treating a paramyxoviridae infection in a subject (e.g., a human) in need thereof, the method comprising administering to the subject a compound described herein. Paramyroviridae viruses include, but are not limited to Nipah virus, Hendra virus, measles, mumps, and parainfluenza virus.

In some embodiments, the viral infection is a human parainfluenza virus, Nipah virus, Hendra virus, measles, or mumps infection.

In some embodiments, the viral infection is a pneumoviridae virus infection. As such, in some embodiments, the present disclosure provides a method of treating a pneumoviridae virus infection in a human in need thereof, the method comprising administering to the human a compound described herein. Pneumoviridae viruses include, but are not limited to, respiratory syncytial virus and human metapneumovirus. In some embodiments, the pneumoviridae virus infection is a respiratory syncytial virus infection. In some embodiments, the pneumoviridae virus infection is human metapneumovirus infection.

In some embodiments, the present disclosure provides a compound described herein, for use in the treatment of a pneumoviridae virus infection in a human in need thereof. In some embodiments, the pneumoviridae virus infection is a respiratory syncytial virus infection. In some embodiments, the pneumoviridae virus infection is human metapneumovirus infection.

In some embodiments, the present disclosure provides methods for treating a RSV infection in a human in need thereof, the method comprising administering to the human a compound described herein. In some embodiments, the human is suffering from a chronic respiratory syncytial viral infection. In some embodiments, the human is acutely infected with RSV.

In some embodiments, a method of inhibiting RSV replication is provided, wherein the method comprises administering to a human in need thereof, a compound described herein, wherein the administration is by inhalation.

In some embodiments, the present disclosure provides a method for reducing the viral load associated with RSV infection, wherein the method comprises administering to a human infected with RSV a compound described herein.

In some embodiments, the viral infection is a picornaviridae virus infection. As such, in some embodiments, the present disclosure provides a method of treating a picornaviridae virus infection in a human in need thereof, the method comprising administering to the human a compound described herein. Picornaviridae viruses are enteroviruses causing a heterogeneous group of infections including herpangina, aseptic meningitis, a common-cold-like syndrome (human rhinovirus infection), a non-paralytic poliomyelitis-like syndrome, epidemic pleurodynia (an acute, febrile, infectious disease generally occurring in epidemics), hand-foot-mouth syndrome, pediatric and adult pancreatitis and serious myocarditis. In some embodiments, the picornaviridae virus infection is human rhinovirus infection (HRV). In some embodiments, the picornaviridae virus infection is HRV-A, HRV-B, or HRV-C infection.

In some embodiments, the viral infection is selected from Coxsackie A virus infection, Coxsackie A virus infection, enterovirus D68 infection, enterovirus B69 infection, enterovirus D70 infection, enterovirus A71 infection, and poliovirus infection.

In some embodiments, the present disclosure provides a compound, for use in the treatment of a picornaviridae virus infection in a human in need thereof. In some embodiments, the picornaviridae virus infection is human rhinovirus infection.

In some embodiments, the viral infection is a flaviviridae virus infection. As such, in some embodiments, the present disclosure provides a method of treating a flaviviridae virus infection in a human in need thereof, the method comprising administering to the human a compound described herein. Representative flaviviridae viruses include, but are not limited to, dengue, Yellow fever, West Nile, Zika, Japanese encephalitis virus, and Hepatitis C (HCV). In some embodiments, the flaviviridae virus infection is a dengue virus infection. In some embodiments, the flaviviridae virus infection is a yellow fever virus infection. In some embodiments, the flaviviridae virus infection is a West Nile virus infection. In some embodiments, the flaviviridae virus infection is a zika virus infection. In some embodiments, the flaviviridae virus infection is a Japanese encephalitis virus infection. In some embodiments, the flaviviridae virus infection is a hepatitis C virus infection.

In some embodiments, the flaviviridae virus infection is a dengue virus infection, yellow fever virus infection, West Nile virus infection, tick borne encephalitis, Kunjin Japanese encephalitis, St. Louis encephalitis, Murray valley encephalitis, Omsk hemorrhagic fever, bovine viral diarrhea, zika virus infection, or a HCV infection.

In some embodiments, the present disclosure provides use of a compound described herein for treatment of a flaviviridae virus infection in a human in need thereof. In some embodiments, the flaviviridae virus infection is a dengue virus infection. In some embodiments, the flaviviridae virus infection is a yellow fever virus infection. In some embodiments, the flaviviridae virus infection is a West Nile virus infection. In some embodiments, the flaviviridae virus infection is a zika virus infection. In some embodiments, the flaviviridae virus infection is a hepatitis C virus infection.

In some embodiments, the viral infection is a filoviridae virus infection. As such, in some embodiments, provided herein is a method of treating a filoviridae virus infection in a human in need thereof, the method comprising administering to the human a compound described herein. Representative filoviridae viruses include, but are not limited to, ebola (variants Zaire, Bundibugio, Sudan, Tai forest, or Reston) and marburg. In some embodiments, the filoviridae virus infection is an ebola virus infection. In some embodiments, the filoviridae virus infection is a marburg virus infection.

In some embodiments, the present disclosure provides a compound for use in the treatment of a filoviridae virus infection in a human in need thereof. In some embodiments, the filoviridae virus infection is an ebola virus infection. In some embodiments, the filoviridae virus infection is a marburg virus infection.

In some embodiments, the viral infection is a coronavirus infection. As such, in some embodiments, provided herein is a method of treating a coronavirus infection in a human in need thereof, wherein the method comprises administering to the human a compound described herein. In some embodiments, the coronavirus infection is a Severe Acute Respiratory Syndrome (SARS-CoV) infection, Middle Eastern Respiratory Syndrome (MERS) infection, SARS-CoV-2 infection, other human coronavirus (229E, NL63, OC43, HKU1, or WIV 1) infections, zoonotic coronavirus (PEDV or HKU CoV isolates such as HKU3, HKU5, or HKU9) infections. In some embodiments, the viral infection is a Severe Acute Respiratory Syndrome (SARS) infection. In some embodiments, the viral infection is a Middle Eastern Respiratory Syndrome (MERS) infection. In some embodiments, the viral infection is SARS-CoV-2 infection. In some embodiments, the viral infection is a zoonotic coronavirus infection, In some embodiments, the viral infection is caused by a virus having at least 70% sequence homology to a viral polymerase selected from SARS-CoV polymerase, MERS-CoV polymerase and SARS-CoV-2. In some embodiments, the viral infection is caused by a virus having at least 80% sequence homology to a viral polymerase selected from SARS-CoV polymerase, MERS-CoV polymerase and SARS-CoV-2. In some embodiments, the viral infection is caused by a virus having at least 90% sequence homology to a viral polymerase selected from SARS-CoV polymerase, MERS-CoV polymerase and SARS-CoV-2. In some embodiments, the viral infection is caused by a virus having at least 95% sequence homology to a viral polymerase selected from SARS-CoV polymerase, MERS-CoV polymerase and SARS-CoV-2.

In some embodiments, the viral infection is caused by a variant of SARS-CoV-2, for example by the B.1.1.7 variant (the UK variant), B.1.351 variant (the South African variant), P.1 variant (the Brazil variant), B.1.1.7 with E484K variant, B.1.1.207 variant, B.1.1.317 variant, B.1.1.318 variant, B.1.429 variant, B.1.525 variant, or P.3 variant. In some embodiments, the viral infection is caused by the B.1.1.7 variant of SARS-CoV-2. In some embodiments, the viral infection is caused by the B.1.351 variant of SARS-CoV-2. In some embodiments, the viral infection is caused by the P.1 variant of SARS-CoV-2.

In some embodiments, the present disclosure provides a compound for use in the treatment of a coronavirus virus infection in a human in need thereof. In some embodiments, the coronavirus infection is a Severe Acute Respiratory Syndrome (SARS) infection, Middle Eastern Respiratory Syndrome (MERS) infection, SARS-CoV-2 infection, other human coronavirus (229E, NL63, OC43, HKU1, or WIV1) infections, and zoonotic coronavirus (PEDV or HKU CoV isolates such as HKU3, HKU5, or HKU9) infections. In some embodiments, the viral infection is a Severe Acute Respiratory Syndrome (SARS) infection. In some embodiments, the viral infection is a Middle Eastern Respiratory Syndrome (MERS) infection. In some embodiments, the viral infection is SARS-CoV-2 infection (COVID19).

In some embodiments, the viral infection is a coronavirus infection. In some embodiments, the coronavirus is an alpha coronavirus, a beta coronavirus, a gamma coronavirus, a delta coronavirus, an epsilon coronavirus, an eta coronavirus, an iota coronavirus, a kappa coronavirus, an omicron coronavirus, a zeta coronavirus, or a mu coronavirus.

In some embodiments, the coronavirus infection is an alpha coronavirus infection. In some embodiments, the alpha coronavirus is feline coronavirus (FCoV), FCoV-II, transmissible gastroenteritis virus (TGEV), porcine respiratory coronavirus (PRCV), canine coronavirus (CCoV), CCoV-II, CRCoV, human coronavirus229E (HCoV-229E), human coronavirus NL63 (HCoV-NL63), porcine epidemic diarrhea virus (PEDV), swine acute diarrhea syndrome-coronavirus (SADS-CoV), bat coronaviruses (Bat CoV), or FRCoV.

In some embodiments, the coronavirus infection is a beta coronavirus infection. In some embodiments, the beta coronavirus is human coronavirus OC43 (HCoV-OC43), human coronavirus HKU-1 (HCoV-HKU1), human enteric coronavirus-4408 (HECoV-4408), bovine coronavirus (BCoV), BCoV-like CoVs, BCoV-like CoVs, canine respiratory coronavirus (CRCoV), equine coronavirus (ECoV), porcine hemagglutinating encephalomyelitis virus (PHEV), murine hepatitis virus (MHV), Middle East respiratory syndrome-related coronavirus (MERS-CoV), severe acute respiratory syndrome coronavirus (SARS-CoV), severe acute respiratory syndrome coronavirus 2 (SARS-CoV-2), bat coronaviruses (Bat CoV), rabbit coronavirus (RbCoV), or bubaline coronavirus (BuCoV).

In some embodiments, the beta coronavirus is a sarbecovirus. In some embodiments, the sarbecovirus is Longquan 140, Pangolin-CoV, RmYN02, RaTG13, CoVZC45, CoVZXC21, GX-P4L, RshSTT182, RshSTT200, RacCS203, Rc-o319, RpYN06, or PrC31.

In some embodiments, the coronavirus infection is a gamma coronavirus infection. In some embodiments, the gamma coronavirus is infectious bronchitis virus (IBV), turkey coronavirus (TCoV), bottlenose whale coronavirus (BdCoV), beluga whale coronavirus (BWCoV), or PhCoV.

In some embodiments, the coronavirus infection is a delta coronavirus infection. In some embodiments, the delta coronavirus is a porcine deltacoronavirus (PDCoV).

In some embodiments, the coronavirus infection is an omicron coronavirus infection. In some embodiments, the omicron coronavirus is WA1 (lineage A), BF.7, BQ.1, XBB.1.5, CH.1.1, XBF, XBB.1.16, or XBB.1.9.1.

In some embodiments, the viral infection is an arenaviridae virus infection. As such, in some embodiments, the disclosure provides a method of treating an arenaviridae virus infection in a human in need thereof, the method comprising administering to the human a compound described herein. In some embodiments, the arenaviridae virus infection is a Lassa infection or a Junin infection.

In some embodiments, the present disclosure provides a compound for use in the treatment of an arenaviridae virus infection in a human in need thereof. In some embodiments, the arenaviridae virus infection is a Lassa infection or a Junin infection.

In some embodiments, the viral infection is an orthomyxovirus infection, for example, an influenza virus infection. In some embodiments, the viral infection is an influenza virus A, influenza virus B, or influenza virus C infection.

In some embodiments, the viral infection is a poxvirus infection. In some embodiments, the poxvirus infection is an orthopox virus infection. In some embodiments, the poxvirus infection is a camelpox virus infection, cowpox virus infection, ectromelia virus infection, horsepox virus infection, monkeypox virus infection, raccoonpox virus infection, skunkpox virus infection, taterapox virus infection, uasin gishu virus infection, vaccinia virus infection, variola virus infection, or volepox virus infection.

In some embodiments, the poxvirus infection is a vaccinia virus infection.

In some embodiments, the poxvirus infection is a monkeypox virus infection. The methods described herein can be used to treat or prevent an infection caused by any strain of monkeypox virus. In some embodiments, the poxvirus infection is caused by a West African strain of monkeypox virus. In some embodiments, the poxvirus infection is caused by a Congo Basin strain of monkeypox virus.

In some embodiments, the poxvirus infection is a parapoxvirus infection. In some embodiments, the poxvirus infection is bovine papular stomatitis virus infection, orf virus infection, pseudocowpox virus infection, parapoxvirus of red deer infection, or squirrel parapoxvirus infection. In some embodiments, the poxvirus infection is camel contagious ecthyma (Ausdyk) virus infection, chamois contagious ecthyma virus infection, parapoxvirus of reindeer virus infection, or sealpox virus infection.

In some embodiments, the poxvirus infection is a molluscipoxvirus infection. In some embodiments, the poxvirus infection is a molluscum contagiosum infection.

In some embodiments, the poxvirus infection is a yatapoxvirus infection. In some embodiments, the poxvirus infection is a Tanapox, Yaba-like disease virus infection or yaba monkey tumor virus infection.

In some embodiments, the poxvirus infection is a capripoxvirus infection. In some embodiments, the poxvirus infection is a sheeppox virus infection, goatpox virus infection, or lumpy skin disease virus infection.

In some embodiments, the poxvirus infection is a suipoxvirus infection. In some embodiments, the poxvirus infection is a swinepox virus infection.

In some embodiments, the poxvirus infection is a leporipoxvirus infection. In some embodiments, the poxvirus infection is a myxoma virus infection, shope fibroma virus (rabbit fibroma) infection, squirrel fibroma virus infection, or hare fibroma virus infection.

In some embodiments, the poxvirus infection is an avipoxvirus infection. In some embodiments, the poxvirus infection is canarypox virus infection, fowlpox virus infection, juncopox virus infection, mynahpox virus infection, pigeonpox virus infection, psittacinepox virus infection, quailpox virus infection, sparrowpox virus infection, starlingpox virus infection, or turkeypox virus infection. In some embodiments, the poxvirus infection is crowpox virus infection, peacockpox virus infection, or penguinpox virus infection.

The compounds described herein can also be used in combination with one or more additional therapeutic agents. As such, also provided herein are methods of treatment of a viral infection in a patient in need thereof, wherein the methods comprise administering to the patient a compound described herein and a therapeutically effective amount of one or more additional therapeutic or prophylactic agents.

In some embodiments, the additional therapeutic agent is an antiviral agent. Any suitable antiviral agent can be used in the methods described herein.

In some embodiments, the additional therapeutic agent a 2,5-Oligoadenylate synthetase stimulator, 5-HT 2a receptor antagonist, 5-Lipoxygenase inhibitor, ABL family tyrosine kinase inhibitor, Abl tyrosine kinase inhibitor, Acetaldehyde dehydrogenase inhibitor, Acetyl CoA carboxylase inhibitor, Actin antagonist, Actin modulator, Activity-dependent neuroprotector modulator, Adenosine A3 receptor agonist, Adrenergic receptor antagonist, Adrenomedullin ligand, Adrenomedullin ligand inhibitor, Advanced glycosylation product receptor antagonist, Advanced glycosylation product receptor modulator, AKT protein kinase inhibitor, Alanine proline rich secreted protein stimulator, Aldose reductase inhibitor, Alkaline phosphatase stimulator, Alpha 2 adrenoceptor antagonist, Alpha 2B adrenoceptor agonist, AMP activated protein kinase stimulator, AMPA receptor modulator, Amyloid protein deposition inhibitor, Androgen receptor antagonist, Angiotensin II AT-1 receptor antagonist, Angiotensin II AT-2 receptor agonist, Angiotensin II receptor modulator, Angiotensin converting enzyme 2 inhibitor, Angiotensin converting enzyme 2 modulator, Angiotensin converting enzyme 2 stimulator, Angiotensin receptor modulator, Annexin A5 stimulator, Anoctamin 1 inhibitor, Anti-coagulant, Anti-histamine, Anti-hypoxic, Anti-thrombotic, API transcription factor modulator, Apelin receptor agonist, APOA1 gene stimulator, Apolipoprotein A1 agonist, Apolipoprotein B antagonist, Apolipoprotein B modulator, Apolipoprotein C₃ antagonist, Aryl hydrocarbon receptor agonist, Aryl hydrocarbon receptor antagonist, ATP binding cassette transporter B5 modulator, Axl tyrosine kinase receptor inhibitor, Bactericidal permeability protein inhibitor, Basigin inhibitor, Basigin modulator, BCL2 gene inhibitor, BCL2L11 gene stimulator, Bcr protein inhibitor, Beta 1 adrenoceptor modulator, Beta 2 adrenoceptor agonist, Beta adrenoceptor agonist, Beta-arrestin stimulator, Blood clotting modulator, BMP10 gene inhibitor, BMP15 gene inhibitor, Bone morphogenetic protein-10 ligand inhibitor, Bone morphogenetic protein-15 ligand inhibitor, Bradykinin B2 receptor antagonist, Brain derived neurotrophic factor ligand, Bromodomain containing protein 2 inhibitor, Bromodomain containing protein 4 inhibitor, Btk tyrosine kinase inhibitor, C-reactive protein modulator, Ca2+ release activated Ca2+ channel 1 inhibitor, Cadherin-5 modulator, Calcium activated chloride channel inhibitor, Calcium channel modulator, Calpain-I inhibitor, Calpain-II inhibitor, Calpain-IX inhibitor, Cannabinoid CB2 receptor agonist, Cannabinoid receptor modulator, Casein kinase II inhibitor, CASP8-FADD-like regulator inhibitor, Caspase inhibitor, Catalase stimulator, CCL26 gene inhibitor, CCR2 chemokine antagonist, CCR5 chemokine antagonist, CD11a agonist, CD122 agonist, CD3 antagonist, CD4 agonist, CD40 ligand, CD40 ligand modulator, CD40 ligand receptor agonist, CD40 ligand receptor modulator, CD49d agonist, CD70 antigen modulator, CD73 agonist, CD73 antagonist, CD95 antagonist, CFTR inhibitor, CGRP receptor antagonist, Chemokine receptor-like 1 agonist, Chloride channel inhibitor, Chloride channel modulator, Cholera enterotoxin subunit B inhibitor, Cholesterol ester transfer protein inhibitor, Collagen modulator, Complement C1s subcomponent inhibitor, Complement C₃ inhibitor, Complement C₅ factor inhibitor, Complement C₅a factor inhibitor, Complement Factor H stimulator, Complement cascade inhibitor, Complement factor C₂ inhibitor, Complement factor D inhibitor, Connective tissue growth factor ligand inhibitor, Coronavirus nucleoprotein modulator, Coronavirus small envelope protein modulator, Coronavirus spike glycoprotein inhibitor, Coronavirus spike glycoprotein modulator, COVID19 envelope small membrane protein modulator, COVID19 non structural protein 8 modulator, COVID19 nucleoprotein modulator, COVID19 Protein 3a inhibitor, COVID19 replicase polyprotein 1a inhibitor, COVID19 replicase polyprotein 1a modulator, COVID19 replicase polyprotein lab inhibitor, COVID19 replicase polyprotein lab modulator, COVID19 Spike glycoprotein inhibitor, COVID19 Spike glycoprotein modulator, COVID19 structural glycoprotein modulator, CRF-2 receptor agonist, CSF-1 agonist, CSF-1 antagonist, CX3CR1 chemokine antagonist, CXC10 chemokine ligand inhibitor, CXC5 chemokine ligand inhibitor, CXCL1 gene modulator, CXCL2 gene modulator, CXCL3 gene modulator, CXCR1 chemokine antagonist, CXCR2 chemokine antagonist, CXCR4 chemokine antagonist, Cyclin D1 inhibitor, Cyclin E inhibitor, Cyclin-dependent kinase-1 inhibitor, Cyclin-dependent kinase-2 inhibitor, Cyclin-dependent kinase-5 inhibitor, Cyclin-dependent kinase-7 inhibitor, Cyclin-dependent kinase-9 inhibitor, Cyclooxygenase 2 inhibitor, Cyclooxygenase inhibitor, Cysteine protease inhibitor, Cytochrome P450 3A4 inhibitor, Cytokine receptor antagonist, Cytotoxic T lymphocyte protein gene modulator, Cytotoxic T-lymphocyte protein-4 inhibitor, Cytotoxic T-lymphocyte protein-4 stimulator, Dehydrogenase inhibitor, Dehydropeptidase-1 modulator, Deoxyribonuclease I stimulator, Deoxyribonuclease gamma stimulator, Deoxyribonuclease stimulator, Dihydroceramide delta 4 desaturase inhibitor, Dihydroorotate dehydrogenase inhibitor, Dipeptidyl peptidase I inhibitor, Dipeptidyl peptidase III inhibitor, Diuretic, DNA binding protein inhibitor, DNA methyltransferase inhibitor, Dopamine transporter inhibitor, E selectin antagonist, Ecto NOX disulfide thiol exchanger 2 inhibitor, EGFR gene inhibitor, Elongation factor 1 alpha 2 modulator, Endoplasmin modulator, Endoribonuclease DICER modulator, Endothelin ET-A receptor antagonist, Epidermal growth factor receptor antagonist, Estrogen receptor beta agonist, Estrogen receptor modulator, Eukaryotic initiation factor 4A-I inhibitor, Exo-alpha sialidase modulator, Exportin 1 inhibitor, Factor Ia modulator, Factor IIa modulator, Factor VII antagonist, Factor Xa antagonist, Factor Ma antagonist, FGF receptor antagonist, FGF-1 ligand, FGF-1 ligand inhibitor, FGF-2 ligand inhibitor, FGF1 receptor antagonist, FGF2 receptor antagonist, FGF3 receptor antagonist, Flt3 tyrosine kinase inhibitor, Fractalkine ligand inhibitor, Free fatty acid receptor 2 agonist, Free fatty acid receptor 3 agonist, furin inhibitors, Fyn tyrosine kinase inhibitor, FYVE finger phosphoinositide kinase inhibitor, G-protein coupled bile acid receptor 1 agonist, GABA A receptor modulator, Galectin-3 inhibitor, Gamma-secretase inhibitor, GDF agonist, Gelsolin stimulator, Glial cell neurotrophic factor ligand, Glucocorticoid receptor agonist, Glutathione peroxidase stimulator, GM-CSF ligand inhibitor, GM-CSF receptor agonist, GM-CSF receptor modulator, Griffithsin modulator, Growth regulated protein alpha ligand inhibitor, Grp78 calcium binding protein inhibitor, Heat shock protein HSP90 alpha inhibitor, Heat shock protein HSP90 beta inhibitor, Heat shock protein inhibitor, Heat shock protein stimulator, Hemagglutinin modulator, Hemoglobin modulator, Hemolysin alpha inhibitor, Heparanase inhibitor, Heparin agonist, Hepatitis B structural protein inhibitor, Hepatitis C virus NS5B polymerase inhibitor, HIF prolyl hydroxylase inhibitor, HIF prolyl hydroxylase-2 inhibitor, High mobility group protein B1 inhibitor, Histamine H1 receptor antagonist, Histamine H2 receptor antagonist, Histone deacetylase-6 inhibitor, Histone inhibitor, HIV protease inhibitor, HIV-1 gp120 protein inhibitor, HIV-1 protease inhibitor, HIV-1 reverse transcriptase inhibitor, HLA class I antigen modulator, HLA class II antigen modulator, Host cell factor modulator, Hsp 90 inhibitor, Human papillomavirus E6 protein modulator, Human papillomavirus E7 protein modulator, Hypoxia inducible factor inhibitor gene inhibitor, Hypoxia inducible factor-2 alpha modulator, I-kappa B kinase inhibitor, I-kappa B kinase modulator, ICAM-1 stimulator, IgG receptor FcRn large subunit p51 modulator, IL-12 receptor antagonist, IL-15 receptor agonist, IL-15 receptor modulator, IL-17 antagonist, IL-18 receptor accessory protein antagonist, IL-2 receptor agonist, IL-22 agonist, IL-23 antagonist, IL-6 receptor agonist, IL-6 receptor antagonist, IL-6 receptor modulator, IL-7 receptor agonist, IL-8 receptor antagonist, IL12 gene stimulator, IL8 gene modulator, Immunoglobulin G modulator, Immunoglobulin G1 agonist, Immunoglobulin G1 modulator, Immunoglobulin agonist, Immunoglobulin gamma Fc receptor I modulator, Immunoglobulin kappa modulator, Inosine monophosphate dehydrogenase inhibitor, Insulin sensitizer, Integrin agonist, Integrin alpha-4/beta-7 antagonist, Integrin alpha-V/beta-1 antagonist, Integrin alpha-V/beta-6 antagonist, Interferon agonist, Interferon alpha 14 ligand, Interferon alpha 2 ligand, Interferon alpha 2 ligand modulator, Interferon alpha ligand, Interferon alpha ligand inhibitor, Interferon alpha ligand modulator, Interferon beta ligand, Interferon gamma ligand inhibitor, Interferon gamma receptor agonist, Interferon gamma receptor antagonist, Interferon receptor modulator, Interferon type I receptor agonist, Interleukin 17A ligand inhibitor, Interleukin 17F ligand inhibitor, Interleukin 18 ligand inhibitor, Interleukin 22 ligand, Interleukin-1 beta ligand inhibitor, Interleukin-1 beta ligand modulator, Interleukin-1 ligand inhibitor, Interleukin-2 ligand, Interleukin-29 ligand, Interleukin-6 ligand inhibitor, Interleukin-7 ligand, Interleukin-8 ligand inhibitor, IRAK-4 protein kinase inhibitor, JAK tyrosine kinase inhibitor, Jak1 tyrosine kinase inhibitor, Jak2 tyrosine kinase inhibitor, Jak3 tyrosine kinase inhibitor, Jun N terminal kinase inhibitor, Jun N terminal kinase modulator, Kallikrein modulator, Kelch like ECH associated protein 1 modulator, Kit tyrosine kinase inhibitor, KLKB1 gene inhibitor, Lactoferrin stimulator, Lanosterol-14 demethylase inhibitor, Lck tyrosine kinase inhibitor, Leukocyte Ig like receptor A4 modulator, Leukocyte elastase inhibitor, Leukotriene BLT receptor antagonist, Leukotriene D4 antagonist, Leukotriene receptor antagonist, Listeriolysin stimulator, Liver X receptor antagonist, Low molecular weight heparin, Lung surfactant associated protein B stimulator, Lung surfactant associated protein D modulator, Lyn tyrosine kinase inhibitor, Lyn tyrosine kinase stimulator, Lysine specific histone demethylase 1 inhibitor, Macrophage migration inhibitory factor inhibitor, Mannan-binding lectin serine protease inhibitor, Mannan-binding lectin serine protease-2 inhibitor, MAO B inhibitor, MAP kinase inhibitor, MAPK gene modulator, Matrix metalloprotease modulator, Maxi K potassium channel inhibitor, MCL1 gene inhibitor, MEK protein kinase inhibitor, MEK-1 protein kinase inhibitor, Melanocortin MCI receptor agonist, Melanocortin MC3 receptor agonist, Metalloprotease-12 inhibitor, METTL3 gene inhibitor, Moesin inhibitor, Moesin modulator, Monocyte chemotactic protein 1 ligand inhibitor, Monocyte differentiation antigen CD14 inhibitor, mRNA cap guanine N₇ methyltransferase modulator, mTOR complex 1 inhibitor, mTOR complex 2 inhibitor, mTOR inhibitor, Mucolipin modulator, Muscarinic receptor antagonist, Myeloperoxidase inhibitor, NACHT LRR PYD domain protein 3 inhibitor, NAD synthase modulator, NADPH oxidase inhibitor, Neuropilin 2 modulator, Neuroplastin inhibitor, NFE2L2 gene stimulator, NK cell receptor agonist, NK1 receptor antagonist, NMDA receptor antagonist, NMDA receptor epsilon 2 subunit inhibitor, Non receptor tyrosine kinase TYK2 antagonist, Non-nucleoside reverse transcriptase inhibitor, Nuclear erythroid 2-related factor 2 stimulator, Nuclear factor kappa B inhibitor, Nuclear factor kappa B modulator, Nuclease stimulator, Nucleolin inhibitor, Nucleoprotein inhibitor, Nucleoprotein modulator, Nucleoside reverse transcriptase inhibitor, Opioid receptor agonist, Opioid receptor antagonist, Opioid receptor mu modulator, Opioid receptor sigma antagonist 1, Ornithine decarboxylase inhibitor, Outer membrane protein inhibitor, OX40 ligand, p38 MAP kinase alpha inhibitor, p38 MAP kinase inhibitor, p38 MAP kinase modulator, p53 tumor suppressor protein stimulator, Palmitoyl protein thioesterase1 inhibitor, Papain inhibitor, PARP inhibitor, PARP modulator, PDE 10 inhibitor, PDE 3 inhibitor, PDE 4 inhibitor, PDGF receptor alpha antagonist, PDGF receptor antagonist, PDGF receptor beta antagonist, Peptidyl-prolyl cis-trans isomerase A inhibitor, Peroxiredoxin 6 modulator, PGD2 antagonist, PGI2 agonist, P-glycoprotein inhibitor, Phosphoinositide 3-kinase inhibitor, Phosphoinositide-3 kinase delta inhibitor, Phosphoinositide-3 kinase gamma inhibitor, Phospholipase A2 inhibitor, Plasma kallikrein inhibitor, Plasminogen activator inhibitor 1 inhibitor, Platelet inhibitor, Platelet glycoprotein VI inhibitor, Polo-like kinase 1 inhibitor, Poly ADP ribose polymerase 1 inhibitor, Poly ADP ribose polymerase 2 inhibitor, Polymerase cofactor VP35 inhibitor, PPAR alpha agonist, Progesterone receptor agonist, Programmed cell death protein 1 modulator, Prolyl hydroxylase inhibitor, Prostaglandin E synthase-1 inhibitor, Protease inhibitor, Proteasome inhibitor, Protein arginine deiminase IV inhibitor, Protein tyrosine kinase inhibitor, Protein tyrosine phosphatase beta inhibitor, Protein tyrosine phosphatase-2C inhibitor, Proto-oncogene Mas agonist, Purinoceptor antagonist, Raf protein kinase inhibitor, RANTES ligand, Ras gene inhibitor, Retinoate receptor responder protein 2 stimulator, Rev protein modulator, Ribonuclease stimulator, RIP-I kinase inhibitor, RNA helicase inhibitor, RNA polymerase inhibitor, RNA polymerase modulator, S phase kinase associated protein 2 inhibitor, SARS coronavirus 3C protease like inhibitor, Serine protease inhibitor, Serine threonine protein kinase ATR inhibitor, Serine threonine protein kinase TBK1 inhibitor, Serum amyloid A protein modulator, Signal transducer CD24 stimulator, Sodium channel stimulator, Sodium glucose transporter-2 inhibitor, Sphingosine kinase 1 inhibitor, Sphingosine kinase 2 inhibitor, Sphingosine kinase inhibitor, Sphingosine-1-phosphate receptor-1 agonist, Sphingosine-1-phosphate receptor-I antagonist, Sphingosine-1-phosphate receptor-1 modulator, Sphingosine-1-phosphate receptor-5 agonist, Sphingosine-I-phosphate receptor-5 modulator, Spike glycoprotein inhibitor, Src tyrosine kinase inhibitor, STAT-1 modulator, STAT-3 inhibitor, STAT-5 inhibitor, STAT3 gene inhibitor, Stem cell antigen-I inhibitor, Stimulator of interferon genes protein stimulator, Sulfatase inhibitor, Superoxide dismutase modulator, Superoxide dismutase stimulator, Syk tyrosine kinase inhibitor, T cell immunoreceptor Ig ITIM protein inhibitor, T cell receptor agonist, T cell surface glycoprotein CD28 inhibitor, T-cell differentiation antigen CD6 inhibitor, T-cell surface glycoprotein CD8 stimulator, T-cell transcription factor NFAT modulator, Tankyrase-1 inhibitor, Tankyrase-2 inhibitor, Tek tyrosine kinase receptor stimulator, Telomerase modulator, Tetanus toxin modulator, TGF beta receptor antagonist, TGFB2 gene inhibitor, Thymosin beta 4 ligand, Thyroid hormone receptor beta agonist, Tissue factor inhibitor, Tissue plasminogen activator modulator, Tissue plasminogen activator stimulator, TLR agonist, TLR modulator, TLR-2 agonist, TLR-2 antagonist, TLR-3 agonist, TLR-4 agonist, TLR-4 antagonist, TLR-6 agonist, TLR-7 agonist, TLR-7 antagonist, TLR-8 antagonist, TLR-9 agonist, TMPRSS2 gene inhibitor, TNF alpha ligand inhibitor, TNF alpha ligand modulator, TNF binding agent, TNF gene inhibitor, Topoisomerase inhibitor, Transcription factor EB stimulator, Transferrin modulator, Transketolase inhibitor, Translocation associated protein inhibitor, Transmembrane serine protease 2 inhibitor, Transthyretin modulator, TREM receptor 1 antagonist, TRP cation channel C1 modulator, TRP cation channel C6 inhibitor, TRP cation channel V6 inhibitor, Trypsin 1 inhibitor, Trypsin 2 inhibitor, Trypsin 3 inhibitor, Trypsin inhibitor, Tubulin alpha inhibitor, Tubulin beta inhibitor, Tumor necrosis factor 14 ligand inhibitor, TYK2 gene inhibitor, Type I IL-1 receptor antagonist, Tyrosine protein kinase ABL1 inhibitor, Ubiquinol cytochrome C reductase 14 kDa inhibitor, Ubiquitin ligase modulator, Unspecified GPCR agonist, Unspecified cytokine receptor modulator, Unspecified enzyme stimulator, Unspecified gene inhibitor, Unspecified receptor modulator, Urokinase plasminogen activator inhibitor, Vascular cell adhesion protein 1 agonist, Vasodilator, VEGF ligand inhibitor, VEGF receptor antagonist, VEGF-1 receptor antagonist, VEGF-1 receptor modulator, VEGF-2 receptor antagonist, VEGF-3 receptor antagonist, Vimentin inhibitor, Vimentin modulator, VIP receptor agonist, Viral envelope protein inhibitor, Viral protease inhibitor, Viral protease modulator, Viral protein target modulator, Viral ribonuclease inhibitor, Viral structural protein modulator, Vitamin D3 receptor agonist, X-linked inhibitor of apoptosis protein inhibitor, Xanthine oxidase inhibitor, or Zonulin inhibitor.

In some embodiments, the compounds and compositions of the present disclosure may be administered in combination with a Sars-Cov-2 treatment, such as parenteral fluids (including dextrose saline and Ringer's lactate), nutrition, antibiotics (including azithromycin, metronidazole, amphotericin B, amoxicillin/clavulanate, trimethoprim/sulfamethoxazole, R-327 and cephalosporin antibiotics, such as ceftriaxone and cefuroxime), antifungal prophylaxis, fever and pain medication, antiemetic (such as metoclopramide) and/or antidiarrheal agents, vitamin and mineral supplements (including Vitamin K, vitamin D, cholecalciferol, vitamin C and zinc sulfate), anti-inflammatory agents (such as ibuprofen or steroids), corticosteroids such as dexamethasone, methylprednisolone, prednisone, mometasone, immunomodulatory medications (eg interferon), vaccines, and pain medications.

In some embodiments, the additional therapeutic agent is an Abl tyrosine kinase inhibitor, such as radotinib or imatinib.

In some embodiments, the additional therapeutic agent is an acetaldehyde dehydrogenase inhibitor, such as ADX-629.

In some embodiments, the additional therapeutic agent is an adenosine A3 receptor agonist, such as piclidenoson.

In some embodiments, the additional therapeutic agent is an adrenomedullin ligand such as adrenomedullin.

In some embodiments, the additional therapeutic agent is a p38 MAPK+PPAR gamma agonist/insulin sensitizer such as KIN-001.

In some embodiments, the additional therapeutic agent is an aldose reductase inhibitor, such as caficrestat.

In some embodiments, the additional therapeutic agent is an AMPA receptor modulator, such as traneurocin.

In some embodiments, the additional therapeutic agent is an annexin A5 stimulator, such as AP-01 or SY-005.

In some embodiments, the additional therapeutic agent is an anti-coagulant, such as heparins (heparin and low molecular weight heparin), aspirin, apixaban, dabigatran, edoxaban, argatroban, enoxaparin, or fondaparinux.

In some embodiments, the additional therapeutic agent is an androgen receptor antagonist such as bicalutamide, enzalutamide, or pruxelutamide (proxalutamide).

In some embodiments, the additional therapeutic agent is anti-hypoxic, such as trans-sodium crocetinate.

In some embodiments, the additional therapeutic agent is an anti-thrombotic, such as defibrotide, rivaroxaban, alteplase, tirofiban, clopidogrel, prasugrel, bemiparin, bivalirudin, sulodexide, or tenecteplase.

In some embodiments, the additional therapeutic agent is an antihistamine, such as cloroperastine or clemastine.

In some embodiments, the additional therapeutic agent is an apolipoprotein A1 agonist, such as CER-001.

In some embodiments, the additional therapeutic agent is a phospholipase A2 inhibitor, such as icosapent ethyl.

In some embodiments, the additional therapeutic agent is an axl tyrosine kinase receptor inhibitor, such as bemcentinib.

In some embodiments, the additional therapeutic agent is a corticosteroid/beta 2 adrenoceptor agonist, such as budesonide+formoterol fumarate.

In some embodiments, the additional therapeutic agent is a BET bromodomain inhibitor/APOA1 gene stimulator such as apabetalone.

In some embodiments, the additional therapeutic agent is a blood clotting modulator, such as lanadelumab.

In some embodiments, the additional therapeutic agent is a bradykinin B2 receptor antagonist, such as icatibant.

In some embodiments, the additional therapeutic agent is an EGFR gene inhibitor/Btk tyrosine kinase inhibitor, such as abivertinib.

In some embodiments, the additional therapeutic agent is a Btk tyrosine kinase inhibitor, such as ibrutinib or zanubrutinib.

In some embodiments, the additional therapeutic agent is a calpain-III/IX inhibitor, such as BLD-2660.

In some embodiments, the additional therapeutic agent is a Ca2+ release activated Ca2+ channel 1 inhibitor, such as zegocractin (CM-4620).

In some embodiments, the additional therapeutic agent is a cadherin-5 modulator, such as FX-06.

In some embodiments, the additional therapeutic agent is a casein kinase II inhibitor, such as silmitasertib.

In some embodiments, the additional therapeutic agent is a caspase inhibitor, such as emricasan.

In some embodiments, the additional therapeutic agent is a catalase stimulator/superoxide dismutase stimulator, such as MP-1032.

In some embodiments, the additional therapeutic agent is a CCR2 chemokine antagonist/CCR5 chemokine antagonist such as cenicriviroc.

In some embodiments, the additional therapeutic agent is a CCR5 chemokine antagonist, such as maraviroc.

In some embodiments, the additional therapeutic agent is a CD122 agonist/IL-2 receptor agonist, such as bempegaldesleukin.

In some embodiments, the additional therapeutic agent is a CD73 agonist/interferon beta ligand, such as FP-1201.

In some embodiments, the additional therapeutic agent is a cholesterol ester transfer protein inhibitor, such as dalcetrapib.

In some embodiments, the additional therapeutic agent is a Mannan-binding lectin serine protease/complement CIs subcomponent inhibitor/myeloperoxidase inhibitor, such as RLS-0071.

In some embodiments, the additional therapeutic agent is a complement C₅ factor inhibitor/leukotriene BLT receptor antagonist, such as nomacopan.

In some embodiments, the additional therapeutic agent is a complement C₅ factor inhibitor, such as zilucoplan.

In some embodiments, the additional therapeutic agent is a CXCR4 chemokine antagonist, such as motixafortide.

In some embodiments, the additional therapeutic agent is a cytochrome P450 3A4 inhibitor/peptidyl-prolyl cis-trans isomerase A inhibitor, such as alisporivir.

In some embodiments, the additional therapeutic agent is a cysteine protease inhibitor, such as SLV-213.

In some embodiments, the additional therapeutic agent is a dihydroorotate dehydrogenase inhibitor, such as brequinar, RP-7214, or emvododstat.

In some embodiments, the additional therapeutic agent is a dehydropeptidase-1 modulator, such as Metablok.

In some embodiments, the additional therapeutic agent is a dihydroorotate dehydrogenase inhibitor/IL-17 antagonist, such as vidofludimus.

In some embodiments, the additional therapeutic agent is a diuretic, such as an aldosterone antagonist, such as spironolactone.

In some embodiments, the additional therapeutic agent is a deoxyribonuclease I stimulator, such as GNR-039 or dornase alfa.

In some embodiments, the additional therapeutic agent is a NET inhibitor, such as NTR-441.

In some embodiments, the additional therapeutic agent is a dihydroceramide delta 4 desaturase inhibitor/sphingosine kinase 2 inhibitor, such as opaganib.

In some embodiments, the additional therapeutic agent is a DNA methyltransferase inhibitor, such as azacytidine.

In some embodiments, the additional therapeutic agent is an LXR antagonist, such as larsucosterol.

In some embodiments, the additional therapeutic agent is a dipeptidyl peptidase I inhibitor, such as brensocatib.

In some embodiments, the additional therapeutic agent is an elongation factor 1 alpha 2 modulator, such as plitidepsin.

In some embodiments, the additional therapeutic agent is a eukaryotic initiation factor 4A-I inhibitor, such as zotatifin.

In some embodiments, the additional therapeutic agent is an exo-alpha sialidase modulator, such as DAS-181.

In some embodiments, the additional therapeutic agent is an exportin 1 inhibitor, such as selinexor.

In some embodiments, the additional therapeutic agent is a fractalkine ligand inhibitor, such as KAND-567.

In some embodiments, the additional therapeutic agent is a FYVE finger phosphoinositide kinase inhibitor/IL-12 receptor antagonist/IL-23 antagonist, such as apilimod dimesylate.

In some embodiments, the additional therapeutic agent is a GABA A receptor modulator, such as brexanolone.

In some embodiments, the additional therapeutic agent is a glucocorticoid receptor agonist, such as ciclesonide, hydrocortisone, dexamethasone, dexamethasone phosphate, or 101-PGC-005.

In some embodiments, the additional therapeutic agent is a GM-CSF receptor agonist, such as sargramostim.

In some embodiments, the additional therapeutic agent is a GPCR agonist, such as esuberaprost sodium.

In some embodiments, the additional therapeutic agent is a Griffithsin modulator, such as Q-Griffithsin.

In some embodiments, the additional therapeutic agent is a leukotriene D4 antagonist, such as montelukast.

In some embodiments, the additional therapeutic agent is a histamine H1 receptor antagonist, such as ebastine, tranilast, levocetirizine dihydrochloride.

In some embodiments, the additional therapeutic agent is a histamine H2 receptor antagonist, such as famotidine.

In some embodiments, the additional therapeutic agent is a heat shock protein stimulator/insulin sensitizer/PARP inhibitor, such as BGP-15.

In some embodiments, the additional therapeutic agent is a histone inhibitor, such as STC-3141.

In some embodiments, the additional therapeutic agent is a histone deacetylase-6 inhibitor, such as CKD-506.

In some embodiments, the additional therapeutic agent is a HIF prolyl hydroxylase-2 inhibitor, such as desidustat.

In some embodiments, the additional therapeutic agent is an HIF prolyl hydroxylase inhibitor, such as vadadustat.

In some embodiments, the additional therapeutic agent is an IL-8 receptor antagonist, such as reparixin.

In some embodiments, the additional therapeutic agent is an IL-7 receptor agonist, such as CYT-107.

In some embodiments, the additional therapeutic agent is an IL-7 receptor agonist/interleukin-7 ligand, such as efineptakin alfa.

In some embodiments, the additional therapeutic agent is an IL-22 agonist, such as efmarodocokin alfa.

In some embodiments, the additional therapeutic agent is an IL-22 agonist/interleukin 22 ligand, such as F-652.

In some embodiments, the additional therapeutic agent is an integrin alpha-V/beta-1 antagonist/integrin alpha-V/beta-6 antagonist, such as bexotegrast.

In some embodiments, the additional therapeutic agent is an interferon alpha 2 ligand, such as interferon alfa-2b or Virafin.

In some embodiments, the additional therapeutic agent is an interferon beta ligand, such as interferon beta-1a follow-on biologic, interferon beta-1b, or SNG-001.

In some embodiments, the additional therapeutic agent is an interferon receptor modulator, such as peginterferon lambda-i a.

In some embodiments, the additional therapeutic agent is an interleukin-2 ligand, such as aldesleukin.

In some embodiments, the additional therapeutic agent is an IRAK-4 protein kinase inhibitor, such as zimlovisertib.

In some embodiments, the additional therapeutic agent is a JAK inhibitor, for example the additional therapeutic agent is baricitinib, filgotinib, jaktinib, tofacitinib, or nezulcitinib (TD-0903).

In some embodiments, the additional therapeutic agent is a neutrophil elastase inhibitor, such as alvelestat.

In some embodiments, the additional therapeutic agent is a lung surfactant associated protein D modulator, such as AT-100.

In some embodiments, the additional therapeutic agent is a plasma kallikrein inhibitor, such as donidalorsen.

In some embodiments, the additional therapeutic agent is a lysine specific histone demethylase 1/MAO B inhibitor, such as vafidemstat.

In some embodiments, the additional therapeutic agent is a Mannan-binding lectin serine protease inhibitor, such as conestat alfa.

In some embodiments, the additional therapeutic agent is a maxi K potassium channel inhibitor, such as ENA-001.

In some embodiments, the additional therapeutic agent is a MEK protein kinase inhibitor, such as zapnometinib.

In some embodiments, the additional therapeutic agent is a MEK-1 protein kinase inhibitor/Ras gene inhibitor, such as antroquinonol.

In some embodiments, the additional therapeutic agent is a melanocortin MCI receptor agonist, such as PL-8177.

In some embodiments, the additional therapeutic agent is a matrix metalloprotease-12 inhibitor, such as FP-025.

In some embodiments, the additional therapeutic agent is a NACHT LRR PYD domain protein 3 inhibitor, such as dapansutrile, DFV-890, or ZYIL-1.

In some embodiments, the additional therapeutic agent is a NADPH oxidase inhibitor, such as isuzinaxib.

In some embodiments, the additional therapeutic agent is a neuropilin 2 modulator, such as efzofitimod.

In some embodiments, the additional therapeutic agent is an NK1 receptor antagonist, such as aprepitant or tradipitant.

In some embodiments, the additional therapeutic agent is an NMDA receptor antagonist, such as transcrocetin or ifenprodil.

In some embodiments, the additional therapeutic agent is a nuclear factor kappa B inhibitor/p38 MAP kinase inhibitor, such as zenuzolac.

In some embodiments, the additional therapeutic agent is an ornithine decarboxylase inhibitor, such as eflornithine.

In some embodiments, the additional therapeutic agent is an opioid receptor sigma antagonist 1, such as MR-309.

In some embodiments, the additional therapeutic agent is a PGD2 antagonist, such as asapiprant.

In some embodiments, the additional therapeutic agent is a PDGF receptor antagonist/TGF beta receptor antagonist/p38 MAP kinase inhibitor, such as deupirfenidone.

In some embodiments, the additional therapeutic agent is a phospholipase A2 inhibitor, such as varespladib methyl.

In some embodiments, the additional therapeutic agent is a phosphoinositide 3-kinase inhibitor/mTOR complex inhibitor, such as dactolisib.

In some embodiments, the additional therapeutic agent is a phosphoinositide-3 kinase delta/gamma inhibitor, such as duvelisib.

In some embodiments, the additional therapeutic agent is a plasminogen activator inhibitor 1 inhibitor, such as TM-5614.

In some embodiments, the additional therapeutic agent is a protein tyrosine phosphatase beta inhibitor, such as razuprotafib.

In some embodiments, the additional therapeutic agent is a RIP-1 kinase inhibitor, such as DNL-758 or SIR-0365.

In some embodiments, the additional therapeutic agent is a Rev protein modulator, such as obefazimod.

In some embodiments, the additional therapeutic agent is an S phase kinase associated protein 2 inhibitor, such as niclosamide or DWRX-2003.

In some embodiments, the additional therapeutic agent is a signal transducer CD24 stimulator, such as EXO-CD24.

In some embodiments, the additional therapeutic agent is a sodium glucose transporter-2 inhibitor, such as dapagliflozin propanediol.

In some embodiments, the additional therapeutic agent is a sodium channel stimulator, such as solnatide.

In some embodiments, the additional therapeutic agent is a sphingosine-1-phosphate receptor-1 agonist/sphingosine-1-phosphate receptor-5 agonist, such as ozanimod.

In some embodiments, the additional therapeutic agent is a non-steroidal anti-inflammatory drug, such as Ampion.

In some embodiments, the additional therapeutic agent is a superoxide dismutase stimulator, such as avasopasem manganese.

In some embodiments, the additional therapeutic agent is a Syk tyrosine kinase inhibitor, such as fostamatinib disodium.

In some embodiments, the additional therapeutic agent is a Tie2 tyrosine kinase receptor agonist, such as AV-001.

In some embodiments, the additional therapeutic agent is a TGFB2 gene inhibitor, such as trabedersen.

In some embodiments, the additional therapeutic agent is a tissue factor inhibitor, such as AB-201.

In some embodiments, the additional therapeutic agent is a TLR-3 agonist, such as rintatolimod.

In some embodiments, the additional therapeutic agent is a TLR-4 antagonist, such as ApTLR-4FT, EB-05, or eritoran.

In some embodiments, the additional therapeutic agent is a TLR-7/8 antagonist, such as enpatoran.

In some embodiments, the additional therapeutic agent is a TLR-2/6 agonist, such as INNA-051.

In some embodiments, the additional therapeutic agent is a TLR-7 agonist, such as PRTX-007.

In some embodiments, the additional therapeutic agent is a TLR agonist, such as PUL-042.

In some embodiments, the additional therapeutic agent is a TLR-4 agonist, such as REVTx-99.

In some embodiments, the additional therapeutic agent is a TLR-2/4 antagonist, such as VB-201.

In some embodiments, the additional therapeutic agent is a TNF alpha ligand inhibitor, such as pegipanermin.

In some embodiments, the additional therapeutic agent is a type I IL-1 receptor antagonist, such as anakinra.

In some embodiments, the additional therapeutic agent is a TREM receptor 1 antagonist, such as nangibotide.

In some embodiments, the additional therapeutic agent is a trypsin inhibitor, such as ulinastatin.

In some embodiments, the additional therapeutic agent is a tubulin inhibitor such as sabizabulin, CCI-001, PCNT-13, CR-42-24, albendazole, entasobulin, SAR-132885, or ON-24160.

In some embodiments, the additional therapeutic agent is a VIP receptor agonist, such as aviptadil.

In some embodiments, the additional therapeutic agent is a xanthine oxidase inhibitor, such as oxypurinol.

In some embodiments, the additional therapeutic agent is a vasodilator, such as iloprost, epoprostenol (VentaProst), zavegepant, TXA-127, USB-002, ambrisentan, nitric oxide nasal spray (NORS), pentoxifylline, propranolol, RESP301, sodium nitrite, or dipyridamole.

In some embodiments, the additional therapeutic agent is a vitamin D3 receptor agonist, such as cholecalciferol.

In some embodiments, the additional therapeutic agent is a zonulin inhibitor, such as larazotide acetate.

In some embodiments, the additional therapeutic agent is a synthetic retinoid derivative, such as fenretinide.

In some embodiments, the additional therapeutic agent is a glucose metabolism inhibitor such as WP-1122.

In some embodiments, the additional therapeutic agent is AT-H201, 2-deoxy-D-glucose, AD-17002, AIC-649, astodrimer, AZD-1656, bitespiramycin, bucillamine, budesonide, CNM-AgZn-17, Codivir, didodecyl methotrexate, DW-2008S (DW-2008), EDP-1815, EG-009A, Fabencov, Gamunex, genistein, GLS-1200, hzVSF-v13, imidazolyl ethanamide pentandioic acid, IMM-101, MAS-825, MRG-001, Nasitrol, Nylexa, OP-101, OPN-019, Orynotide rhesus theta defensin-1, pyronaridine+artesunate, dapsone, RPH-104, sodium pyruvate, Sulforadex, tafenoquine, TB-006, telacebec, Tempol, TL-895, thimesoral, trimodulin, XC-221, XC-7, zunsemetinib, metformin glycinate, lucinactant, EOM-613, mosedipimod, ivermectin, leflunomide, ibudilast, RBT-9, raloxifene, prothione, gemcabene, or idronoxil.

In some embodiments, the additional therapeutic agent is a CD73 antagonist, such as AK-119.

In some embodiments, the additional therapeutic agent is a CD95 protein fusion, such as asunercept.

In some embodiments, the additional therapeutic agent is a complement factor C2 modulator, such as ARGX-117.

In some embodiments, the additional therapeutic agent is a complement C3 inhibitor, such as NGM-621.

In some embodiments, the additional therapeutic agent is a CXC10 chemokine ligand inhibitor, such as EB-06.

In some embodiments, the additional therapeutic agent is a cytotoxic T-lymphocyte protein-4 fusion protein, such as abatacept

In some embodiments, the additional therapeutic agent is an anti-S. aureus antibody, such as tosatoxumab.

In some embodiments, the additional therapeutic agent is an anti-LPS antibody, such as IMM-124-E.

In some embodiments, the additional therapeutic agent is an adrenomedullin ligand inhibitor, such as enibarcimab.

In some embodiments, the additional therapeutic agent is a basigin inhibitor, such as meplazumab.

In some embodiments, the additional therapeutic agent is a CD3 antagonist, such as foralumab.

In some embodiments, the additional therapeutic agent is a connective tissue growth factor ligand inhibitor, such as pamrevlumab.

In some embodiments, the additional therapeutic agent is a complement C5a factor inhibitor, such as BDB-1 or vilobelimab.

In some embodiments, the additional therapeutic agent is a complement C5 factor inhibitor, such as ravulizumab.

In some embodiments, the additional therapeutic agent is a mannan-binding lectin serine protease-2 inhibitor, such as narsoplimab.

In some embodiments, the additional therapeutic agent is a GM-CSF modulator, such as gimsilumab, namilumab, plonmarlimab, otolimab, or lenzilumab.

In some embodiments, the additional therapeutic agent is a heat shock protein inhibitor/IL-6 receptor antagonist, such as siltuximab.

In some embodiments, the additional therapeutic agent is an IL-6 receptor antagonist, such as clazakizumab, levilimab, olokizumab, tocilizumab, or sirukumab.

In some embodiments, the additional therapeutic agent is an IL-8 receptor antagonist, such as BMS-986253.

In some embodiments, the additional therapeutic agent is an interleukin-1 beta ligand inhibitor, such as canakinumab.

In some embodiments, the additional therapeutic agent is an interferon gamma ligand inhibitor, such as emapalumab.

In some embodiments, the additional therapeutic agent is an anti-ILT7 antibody, such as daxdilimab.

In some embodiments, the additional therapeutic agent is a monocyte differentiation antigen CD14 inhibitor, such as atibuclimab.

In some embodiments, the additional therapeutic agent is a plasma kallikrein inhibitor, such as lanadelumab.

In some embodiments, the additional therapeutic agent is a platelet glycoprotein VI inhibitor, such as glenzocimab.

In some embodiments, the additional therapeutic agent is a T-cell differentiation antigen CD6 inhibitor, such as itolizumab.

In some embodiments, the additional therapeutic agent is a TNF alpha ligand inhibitor/TNF binding agent, such as infliximab.

In some embodiments, the additional therapeutic agent is an anti-LIGHT antibody, such as AVTX-002.

In some embodiments, the additional therapeutic agent is COVID-HIG.

In some embodiments, a compound of the disclosure, or a pharmaceutically acceptable salt thereof, is co-administered with one or more agents useful for the treatment and/or prophylaxis of COVID-19.

Non-limiting examples of such agents include corticosteroids, such as dexamethasone, hydrocortisone, methylprednisolone, or prednisone; interleukin-6 (IL-6) receptor blockers, such as tocilizumab or sarilumab; Janus kinase (JAK) inhibitors, such as baricitinib, ruxolitinib, or tofacitinib; and antiviral agents, such as molnupiravir, sotrovimab, or remdesivir, i.e., 2-Ethylbutyl (2S)-2-{[(S)-{[(2R,3S,4R,5R)-5-(4-aminopyrrolo[2,1-f][1,2,4]triazin-7-yl)-5-cyano-3,4-dihydroxytetrahydrofuran-2-yl] methoxy}(phenoxy)phosphoryl]amino}propanoate:

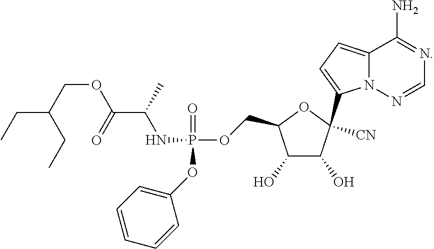 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1]

In further embodiments, a compound of the disclosure, or a pharmaceutically acceptable salt thereof, is co-administered with two or more agents useful for the treatment of COVID-19. Agents useful for the treatment and/or prophylaxis of COVID-19 include but are not limited to a compound of the disclosure and two additional therapeutic agents, such as nirmatrelvir and ritonavir, casirivimab and imdevimab, or ruxolitinib and tofacitinib.

In some embodiments, the additional therapeutic agent is an antiviral agent. In some embodiments, the antiviral agent is an entry inhibitor. In some embodiments, the antiviral agent is a protease inhibitor. In some embodiments, the antiviral agent is an RNA polymerase inhibitor. In some embodiments, the additional therapeutic agent is a RNA-dependent RNA polymerase (RdRp) inhibitor.

In some embodiments, the antiviral agent is selected from angiotensin converting enzyme 2 inhibitors, angiotensin converting enzyme 2 modulators, angiotensin converting enzyme 2 stimulators, angiotensin II AT-2 receptor agonists, angiotensin II AT-2 receptor antagonists, angiotensin II receptor modulators, coronavirus nucleoprotein modulators, coronavirus small envelope protein modulators, coronavirus spike glycoprotein inhibitors, coronavirus spike glycoprotein modulators, COVID19 envelope small membrane protein inhibitors, COVID19 envelope small membrane protein modulators, COVID19 MPro inhibitors, COVID19 non structural protein 8 modulators, COVID19 nucleoprotein inhibitors, COVID19 nucleoprotein modulators, COVID19 protein 3a inhibitors, COVID19 replicase polyprotein 1a inhibitors, COVID19 replicase polyprotein 1a modulators, COVID19 replicase polyprotein lab inhibitors, COVID19 replicase polyprotein 1ab modulators, COVID19 spike glycoprotein inhibitors, COVID19 spike glycoprotein modulators, COVID19 structural glycoprotein modulators, papain inhibitors, protease inhibitors, protease modulators, RNA polymerase inhibitors, RNA polymerase modulators, RNA-dependent RNA polymerase (RdRp) inhibitors, SARS coronavirus 3C protease like inhibitors, 3CLpro/Mpro inhibitors, serine protease inhibitors, transmembrane serine protease 2 inhibitors, transmembrane serine protease 2 modulators, viral envelope protein inhibitors, viral protease inhibitors, viral protease modulators, viral protein target modulators, viral ribonuclease inhibitors, and viral structural protein modulators.

In some embodiments, the additional therapeutic agent is an entry inhibitor. For example, in some embodiments the additional therapeutic agent is an ACE2 inhibitor, a fusion inhibitor, or a protease inhibitor.

In some embodiments, the additional therapeutic agent is an angiotensin converting enzyme 2 inhibitor, such as SBK-001.

In some embodiments, the additional therapeutic agent is an angiotensin converting enzyme 2 modulator, such as neumifil or JN-2019.

In some embodiments, the additional therapeutic agent is an entry inhibitor such as MU-UNMC-1.

In some embodiments, the additional therapeutic agent is an angiotensin converting enzyme 2 stimulator, such as alunacedase alfa.

In some embodiments, the additional therapeutic agent is an angiotensin II AT-2 receptor agonist, such as VP-01.

In some embodiments, the additional therapeutic agent is an ACE II receptor antagonist, such as DX-600.

In some embodiments, the additional therapeutic agent is an angiotensin II receptor modulator, such as TXA-127.

In some embodiments, the additional therapeutic agent is a transmembrane serine protease 2 modulator, such as BC-201.

In some embodiments, the additional therapeutic agent is a viral envelope protein inhibitor, such as MXB-9 or MXB-004.

In some embodiments, the additional therapeutic agent is a vaccine. For example, in some embodiments, the additional therapeutic agent is a DNA vaccine, RNA vaccine, live-attenuated vaccine, inactivated vaccine (i.e., inactivated SARS-CoV-2 vaccine), therapeutic vaccine, prophylactic vaccine, protein-based vaccine, viral vector vaccine, cellular vaccine, or dendritic cell vaccine.

In some embodiments, the additional therapeutic agent is a vaccine such as tozinameran, NVX-CoV2373, elasomeran, KD-414, Janssen COVID-19 Vaccine, Vaxzevria, SCB-2019, AKS-452, VLA-2001, S-268019, MVC-COV1901, mRNA-1273.214, NVX-CoV2515, Covaxin, BBIBP-CorV, GBP-510, mRNA-1273.351+mRNA-1273.617 (SARS-CoV-2 multivalent mRNA vaccine, COVID-19), Ad5-nCoV, Omicron-based COVID-19 vaccine (mRNA vaccine, COVID-19), SARS-CoV-2 Protein Subunit Recombinant Vaccine, Sputnik M, ZyCoV-D, COVID-19 XWG-03, mRNA-1273.529, mRNA-1010, CoronaVac, AZD-2816, Sputnik V, inactivated SARS-CoV-2 vaccine (Vero cell, COVID-19), DS-5670, PHH-IV, INO-4800, UB-612, coronavirus vaccine (whole-virion, inactivated/purified), ReCOV, MT-2766, ARCT-154, SP-0253, CORBEVAX, mRNA-1273.211, ZF-2001, Sputnik Light, recombinant protein vaccine (COVID-19/SARS-CoV-2 infection), VSV vector-based vaccine targeting spike glycoprotein (COVID-19), VLA-2101, GRAd-COV2, VPM-1002, COViran Barekat, Ad5-nCoV-IH, ARCoV, Covax-19, recombinant SARS-CoV-2 vaccine (protein subunit/CHO cell, COVID-19), BBV-154, RAZI Cov Pars, COVID-19 vaccine (inactivated/Vero cells/intramuscular, SARS-CoV-2 infection), COVID-19 vaccine (inactivated, Vero cells/intramuscular), BNT-162b2s01, CIGB-66, mRNA-1273.617, Mycobacterium w, ERUCOV-VAC, AG-0301-COVID19, fakhravac, AV-COVID-19, peptide vaccine (COVID-19), Nanocovax, SARS-CoV-2 vaccine (inactivated/Vero cells/intramuscular, COVID-19), QAZCOVID-IN, S-875670 nasal vaccine, or BNT162b5.

In some embodiments, the additional therapeutic agent is a protease inhibitor. For example, in some embodiments the additional therapeutic agent is a 3C-like cysteine protease inhibitor (3CLpro, also called Main protease, Mpro), a papain-like protease inhibitor (PLpro), serine protease inhibitor, or transmembrane serine protease 2 inhibitor (TMPRSS2).

In some embodiments, the additional therapeutic agent is a 3CLpro/Mpro inhibitor, such as CDI-873, GC-373, GC-376, PBI-0451, UCI-1, DC-402234, DC-402267, RAY-1216, MPI-8, SH-879, SH-580, EDP-235, VV-993, CDI-988, MI-30, nirmatrelvir, ensitrelvir, ASC-11, EDDC-2214, SIM-0417, CDI-45205, COR-803, ALG-097111, TJC-642, CVD-0013943, eravacycline, cynarine, or prexasertib.

In some embodiments, the additional therapeutic agent is a papain-like protease inhibitor (PLpro), such as SBFM-PL4 or GRL-0617.

In some embodiments, the additional therapeutic agent is a SARS-CoV-2 helicase Nsp13 inhibitor, such as EIS-4363.

In some embodiments, the additional therapeutic agent is a SARS-CoV-2 spike (S) and protease modulator, such as ENU-200.

In some embodiments, the additional therapeutic agent is a protease inhibitor, such as ALG-097558 or MRX-18.

In some embodiments, the additional therapeutic agent is a serine protease inhibitor, such as upamostat, nafamostat, camostat mesylate, nafamostat mesylate, or camostat.

In some embodiments, the additional therapeutic agent is a 3CLpro/transmembrane serine protease 2 inhibitor, such as SNB-01 or SNB-02.

In some embodiments, the additional therapeutic agent is a viral protease inhibitor, such as Pan-Corona, Cov-X, or bepridil.

In some embodiments, the additional therapeutic agent is an RNA polymerase inhibitor. For example, in some embodiments, the additional therapeutic agent is an RNA polymerase inhibitor, or a RNA-dependent RNA polymerase (RdRp) inhibitor.

In some embodiments, the additional therapeutic agent is an RNA-dependent RNA polymerase (RdRp) inhibitor, such as remdesivir, NV-CoV-2-R, NV-CoV-1 encapsulated remdesivir, GS-621763, GS-5245, GS-441524, DEP remdesivir, ATV-006, VV-116, LGN-20, CMX-521 and compounds disclosed in WO2022142477, WO2021213288, WO2022047065.

In some embodiments, the additional therapeutic agent is an RNA polymerase inhibitor, such as molnupiravir (EIDD-2801), favipiravir, bemnifosbuvir, sofosbuvir, ASC-10, or galidesivir.

In some embodiments, the additional therapeutic agent is viral entry inhibitor, such as brilacidin.

In some embodiments, the additional therapeutic agent is an antibody that binds to a coronavirus, for example an antibody that binds to SARS or MERS.

In some embodiments, the additional therapeutic agent is an antibody, for example a monoclonal antibody. For example, the additional therapeutic agent is an antibody against SARS-CoV-2, neutralizing nanobodies, antibodies that target the SARS-CoV-2 spike protein, fusion proteins, multispecific antibodies, and antibodies that can neutralize SARS-CoV-2 (SARS-CoV-2 neutralizing antibodies).

In some embodiments, the additional therapeutic agent is an antibody that targets specific sites on ACE2. In some embodiments, the additional therapeutic agent is a polypeptide targeting SARS-CoV-2 spike protein (S-protein).

In some embodiments, the additional therapeutic agent is a SARS-CoV-2 virus antibody.

In some embodiments, the antibody is ABBV-47D11, COVI-GUARD (STI-1499), C144-LS+C135-LS, DXP-604, JMB-2002, LY-CovMab, bamlanivimab (LY-CoV555), S309, SAB-185, etesevimab (CB6), COR-101, JSO16, VNAR, VIR-7832 and/or sotrovimab (VIR-7831), casirivimab+imdevimab (REGN-COV2 or REGN10933+RGN10987), BAT2020, BAT2019, 47D 11, YBSW-015, or PA-001.

In some embodiments, the additional therapeutic agent is STI-9199 (COVI-SHIELD) or AR-701 (AR-703 and AR-720).

In some embodiments, the additional therapeutic agent is BRII-196, BRII-198, ADG-10, ADG-20, ABP-300, BI-767551, CT-P63, JS-026, sotrovimab (GSK-4182136), tixagevimab+cilgavimab (AZD-7442), regdanvimab, SAB-301, AOD-01, plutavimab (COVI-AMG), 9MW-3311 (MW-33), DXP-593, BSVEQAb, anti-SARS-CoV-2 IgY, COVID-EIG, CSL-760, REGN-3048-3051, SARS-CoV-2 monoclonal antibodies (COVID-19, ADM-03820), enuzovimab (HFB-30132A), INM-005, SCTA01, TY-027, XAV-19, amubarvimab+romlusevimab, SCTA-01, bebtelovimab, beludavimab, IBI-0123, IGM-6268. FYB-207, REGN-14256, XVR-011, TB202-3, TB181-36, LQ-050, COVAB-36, MAD-0004J08, STI-2099, or ACV-200-17.

In some embodiments, the additional therapeutic agent is an engineered ACE-2-IgG1-Fc-fusion protein targeting SARS-Cov-2 RBD, such as EU-129, bivalent ACE2-IgG Fc null fusion protein (SI-F019).

In some embodiments, the additional therapeutic agent is an ACE2-Fc receptor fusion protein, such as HLX-71.

In some embodiments, the additional therapeutic agent is ensovibep.

In some embodiments, the additional therapeutic agent is SYZJ-001.

In some embodiments, the additional therapeutic agent is an HIV-1 protease inhibitor, such as ASC-09F (ASC-09+ritonavir) or lopinavir+ritonavir.

In some embodiments, the additional therapeutic agent is a non-nucleoside reverse transcriptase inhibitor, such as elsulfavirine.

In some embodiments, the additional therapeutic agent is a nucleoside reverse transcriptase inhibitor, such as azvudine.

In some embodiments, the additional therapeutic agent is Abbv-990, NED-260, ALG-097431, ENOB-CV-01, EIS-10700, beta-521, SIM-0417, molnupiravir, Pan-Corona, Tollovir, nirmatrelvir+ritonavir (PaxIovid®), favipiravir, GC-376, upamostat, LeSoleil-01, LeSoleil-02+, benfovir, VV-116, VV-993, SNB-01, EDP-235, Cov-X, ensitrelvir, MPI-8, masitinib, ALG-097558, ASC-11, PBI-0451, nafamostat, nafamostat mesylate, CDI-45205, COR-803, ALG-097111, BC-201, SH-879, CDI-873, CDI-988, remdesivir, NV-CoV-2-R, NV-CoV-1 encapsulated remdesivir, NA-831+remdesivir, DEP remdesivir, GS-621763, GS-5245, GLS-5310, bemnifosbuvir, QLS-1128, ASC-10, SBFM-PL4, camostat mesylate, UCI-1, DC-402234, ebselen, SH-580, LeSoleil-01, LeSoleil-02+, MRX-18, MXB-9, MI-09, MI-30, SNB-02, TJC-642, ENU-200, CVD-0013943, GS-441524, bepridil, MXB-004, eravacycline, GRL-0617, camostat, GC-373, nitazoxanide, cynarine, prexasertib, RAY-1216, SACT-COVID-19, MP-18, EIDD-1931, EDDC-2214, nitric oxide, apabetalone, AnQlar, SBK-001, LQ-050, CG-SpikeDown, bamlanivimab, HLX-71, FYB-207, ensovibep, SYZJ-001, EU-129, neumifil, JN-2019, AR-701, vostesyl, PLM-402, PJS-539, CTB-ACE2, TB181-36, TB202-3, ABP-300, XVR-011, MU-UNMC-1, MU-UNMC-2, alunacedase alfa, VP-01, TRV-027, DX-600, TXA-127, mRNA-1273.214, Omicron-based COVID-19 vaccine, NVX-CoV2515, tozinameran, elasomeran, Ad5-nCoV, BBIBP-CorV, CoronaVac, MVC-COV1901, NVX-CoV2373, sotrovimab, Sputnik V, Vaxzevria, ZF-2001, or ZyCoV-D.

It is also possible to combine any compound described herein with one or more additional active therapeutic agents in a unitary dosage form for simultaneous or sequential administration to a patient. The combination therapy may be administered as a simultaneous or sequential regimen. When administered sequentially, the combination may be administered in two or more administrations.

Co-administration of a compound described herein with one or more other active therapeutic agents generally refers to simultaneous or sequential administration of a compound of the invention and one or more other active therapeutic agents, such that therapeutically effective amounts of the compound of the invention and one or more other active therapeutic agents are both present in the body of the patient.

Co-administration includes administration of unit dosages of the compounds described herein before or after administration of unit dosages of one or more other active therapeutic agents, for example, administration of the compounds of the invention within seconds, minutes, or hours of the administration of one or more other active therapeutic agents. For example, a unit dose of a compound of the invention can be administered first, followed within seconds or minutes by administration of a unit dose of one or more other active therapeutic agents. Alternatively, a unit dose of one or more other therapeutic agents can be administered first, followed by administration of a unit dose of a compound of the invention within seconds or minutes. In some cases, it may be desirable to administer a unit dose of a compound described herein first, followed, after a period of hours (e.g., 1-12 hours), by administration of a unit dose of one or more other active therapeutic agents. In other cases, it may be desirable to administer a unit dose of one or more other active therapeutic agents first, followed, after a period of hours (e.g., 1-12 hours), by administration of a unit dose of a compound described herein.

The invention will be described in greater detail by way of specific examples. The following examples are offered for illustrative purposes and are not intended to limit the invention in any manner. Those of skill in the art will readily recognize a variety of non-critical parameters, which can be changed or modified to yield essentially the same results.

### EXAMPLES

The invention is further described in the following examples, which do not limit the scope of the invention described in the claims.

### Example 1. Nonclinical Pharmacokinetic (PK) Studies

Compound 16 orally as well as compound 10 administered showed therapeutic efficacy in multiple SARS-CoV-2 animal models. For human dose selection, the results of animal efficacy studies (effect on most relevant efficacy endpoint, TA and dose administered, and projected plasma levels of compound 1) in mouse, ferret and African green monkeys (AGM) animal models were summarized.

Administration of compound 16 to SARS-CoV-2-infected mice (10 mg/kg BID: AUC₀₋₂₄=10,500 h*ng/ml), ferrets (20 mg/kg QD; AUC₀₋₂₄=28,500 h*ng/ml) and AGM (60 mg/kg QD; AUC₀₋₂₄=25,700 h*ng/ml) for 4 or 5 days resulted in a significant reduction of infectious virus titers in lungs and genomic RNA compared with vehicle-treated animals. The PK exposure at the doses evaluated in these studies are summarized in Table 4. Similarly, in the AGM model oral administration of compound 10 (60 mg/kg QD; AUC₀₋₂₄=18,200 h*ng/ml) or (120 mg/kg QD; AUC₀₋₂₄=36,400 h*ng/ml) significantly reduced SARS-CoV-2 levels in the bronchioalveolar lavage fluid, as early as 2 days of oral dosing. The exposures at the doses evaluated for compound 10 are summarized in Table 5,

TABLE 4
Results of Administration of Compound 16

| | | Compound 1 Plasma PK | | | |
|---|---|---|---|---|---|
| | | Mouse | Ferret | AGM | |
| | | n = 10 | n = 4 | n = 6-8 | |
| | | 10 BID | 20 QD | 60 QD | 120 QD |
| Dose (mg/kg) | | Oral | Oral | Oral | Oral |
| Compound 1 | (μM · h) | 36 | 98 | 88 | 175 |
| AUC₀₋₂₄ | (ng · h/mL) | 10,500 | 28,500 | 25,700 | 51,400 |

TABLE 5
Results of Administration of Compound 10

Compound 1 Plasma PK
AGM
n = 6-8

| | 60 QD | 120 OD |
|---|---|---|
| Dose (mg/kg) | Oral | Oral |

| | | | |
|---|---|---|---|
| Compound 1 | (μM · h) | 62.5 | 125 |
| AUC₀₋₂₄ | (ng · h/mL) | 18,200 | 36,400 |

Based on the results of the nonclinical studies, the efficacious plasma exposure target of compound 1 was established as 18,200-36,400 ng-h/mL (AUC₀₋₂₄).

### Example 2. Clinical Studies

Compound 16 safety and pharmacokinetics (PK) were evaluated in a randomized, blinded, placebo-controlled phase 1 single- and multiple-ascending dose studies in healthy participants. Overall, 48 participants were enrolled across 6 cohorts, 8 per cohort, randomized in a 3:1 ratio (active:placebo) with a median age of 31. Most participants enrolled were male (56%) and not Hispanic or Latino (88%). Approximately even proportions were White (46%) or Black or African American (42%). race. Participants enrolled in Cohorts 1, 2, 3, and 4 received a single 100 mg, 300 mg, 900 mg and 1,600 mg dose of compound 16 fasted, respectively. Participants enrolled in Cohort 5 received twice daily (BID) doses of 500 mg (approximately 12 hours apart) for 5 days fasted (morning dose) or 1-hour before the next meal and 2-hours after the previous meal (evening dose). Participants enrolled in Cohort 6 received once daily (QD) doses of 900 mg for 5 days fasted. For the purpose of dose selection, safety and PK data were reviewed for all scheduled visits for all participants in Cohorts 1-6.

Safety

Overall, administration of compound 16 or placebo was safe and well tolerated. Treatment-emergent adverse events (TEAEs) were reported for 10 of 48 (21%) participants. 9 of 10 participants experienced Grade 1 adverse events (AEs); there was one Grade 2 AE (vertigo not attributed to study drug), and no Grade 3 or higher AEs. There were no serious AEs, no AEs leading to premature discontinuation of study drug, and no deaths. The only AEs reported in more than one participant were headache (3 of 48 participants, 6.3%), and contact dermatitis (2 of 48 participants, 4.2%). The only AEs attributed to study drug was headache (Grade 1) which was reported for 2 of 8 (25%) of participants in Cohort 5 (500 mg BID for 5 days).

Overall, 25 of 48 (52.1%) of participants had graded postbaseline laboratory abnormalities. Most abnormalities were reported for a single participant per group. The most frequently reported graded laboratory abnormality was decreased creatinine clearance, which was reported for 13 of 48 (27%) participants overall and for 6 of 8 (75%) of participants in Cohort 4 (1,600 mg single dose). Graded creatinine clearance decreases were transient and resolved in all participants with repeat testing (12 of 13). One participant with a grade 2 creatinine clearance decrease was lost to follow-up. There was one Grade 3 laboratory abnormality: a participant in Cohort 2 (300 mg single dose) experienced a lipase elevation at Day 3. Their lipase was within normal limits on Days 2 and 5.

There were no clinically relevant changes in vital signs, electrocardiograms (ECGs), isual acuity, or fundoscopic examinations.

Pharmacokinetics

Following oral administration of compound 16, compound 1 was the only circulating species. The plasma half-life of this metabolite was ˜6 hours, supporting twice-daily administration. The twice-daily dosing regimen also provided higher daily exposure (AUC₀₋₂₄), higher C_{trough} concentrations, and lower Cₘₐₓ as compared to once daily dosing.

PK exposures of compound 1 were summarized for 6 dosing regimens of compound 16. Results are shown in Table 6.

TABLE 6
AUC and Cₘₐₓ Exposures of Compound 1

| | Day 1 | Day 1 | Day 5 | Day 5 |
|---|---|---|---|---|
| Dose of | AUC_{0-24 h} | Cₘₐₓ | AUC_{0-24 h} | Cₘₐₓ |
| Compound 16 | (ng/mL*h) | (ng/mL) | (ng/mL*h) | (ng/mL) |
| 350 mg BID for | 22,300 | 2,680 | 30,100 | 3,230 |
| 5 days | | | | |
| 500 mg BID for | 31,800 | 3,820 | 43,000 | 4,620 |
| 5 days | | | | |
| 700 mg QD for 5 | 24,900 | 4,850 | 27,610 | 4,030 |
| days | | | | |
| 700 mg AM and | 36,400* | 4,850 | 30,100 | 3,230 |
| 350 mg PM | | | | |
| (day 1) | | | | |
| 350 mg BID | | | | |
| (days 2-5) | | | | |
| 900 mg QD for 5 | 32,000 | 6,230 | 35,500 | 5,180 |
| days | | | | |
| 1600 mg single | 44,400 | 7,060 | | |
| dose | | | | |

No accumulation after QD and ~35% after BID (steady state concentrations on Day 2);
*exposure predicted using modeling.

The PK exposures were either observed exposures (500 mg BID, 900 mg QD; and 1,600 mg single dose) or extrapolated from observed exposures. The 350 mg BID exposures were extrapolated from observed exposures in Cohort 5 of the above-mentioned study. The 700 mg QD exposures were extrapolated from observed exposures in Cohort 6. The day 1 exposures for the 700 mg AM/350 mg PM dosage were predicted using modeling (nonparametric superpositioning, Phoenix v8.2, using elimination slope parameters observed in Cohort 3, slide 33), and day 5 exposures were extrapolated from Cohort 5. On the mean level, all dosing regimens provided exposures (AUC₀₋₂₄) on day 1 and day 5 within or exceeding the target range 18,200 ng/mL*h to 36,400 h*ng/mL established in Example 1, above.

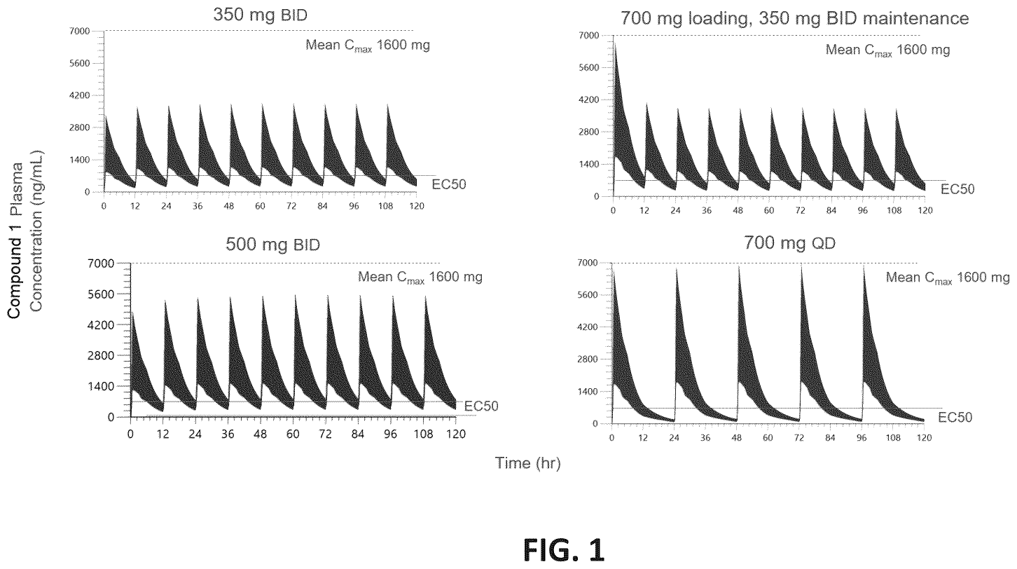

The 350 mg BID for 5 days dose was recommended for evaluation in a phase 3 study for the treatment of COVID-19 in patients at high risk for progression. This dose should provide systemic exposures of compound 1 of 30,100 h*ng/ml (AUC₀₋₂₄, day 5; see Table 6). This dosing regimen also provides a substantial exposure (Cₘₐₓ) margin relative to the 1,600 mg dose (˜2- to 3-fold), where potential drug-related effects on creatine clearance (CrCL) were observed. See FIG. 1.

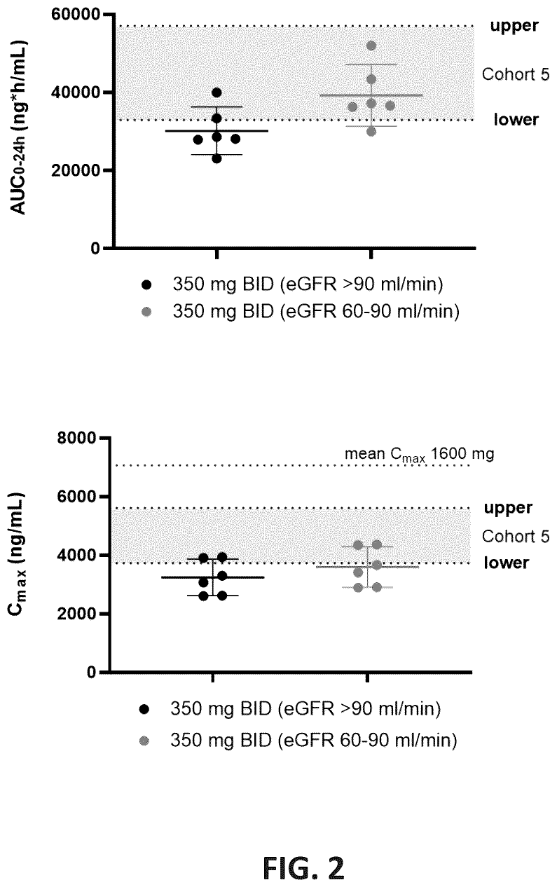

Considering compound 1 is primarily renally eliminated, and circulates in plasma following remdesivir administration, the effects of mild renal impairment on plasma exposure were assessed using data from a phase 1, open-label, parallel-group, single-dose study to evaluate the pharmacokinetics of remdesivir and metabolites in participants with normal renal function and renal impairment. Based on this data, mild renal impairment (eGFR of 60-90 mL/min/1.73 m², estimated using the MDRD equation) was estimated to result in plasma increases of compound 1 of ˜35% (AUC_{inf}) and ˜10% (Cₘₐₓ). By applying these changes to the exposures projected after 350 mg BID dose administration of compound 16 to subjects with normal renal function (estimated using CrCL>90 mL/min), PK exposures (Cₘₐₓ and AUC) in subjects having eGFR ≥60 mL/min/1.73 m² were estimated to be within the range observed after administration of 500 mg BID for 5 days to healthy participants. See FIG. 2. Hence, participants with CrCL >=60 mL/min (as calculated by Cockcroft-Gault) or eGFR ≥60 mL/min/1.73 m² were planned to be enrolled in the phase 3 study without dose adjustment.

### Example 3. Dose Adjustment for Subjects with Impaired Renal Function

Dose adjustment of compound 16 in subjects with impaired renal function was further evaluated using modeling. Plasma and urine PK data from the study of Example 2, in conjunction with plasma and urine data from a phase 1 study of remdesivir in non-COVID-19 participants with full range of renal impairment (normal, mild, moderate, and severe renal impairment, and kidney disease) were utilized in this modeling.

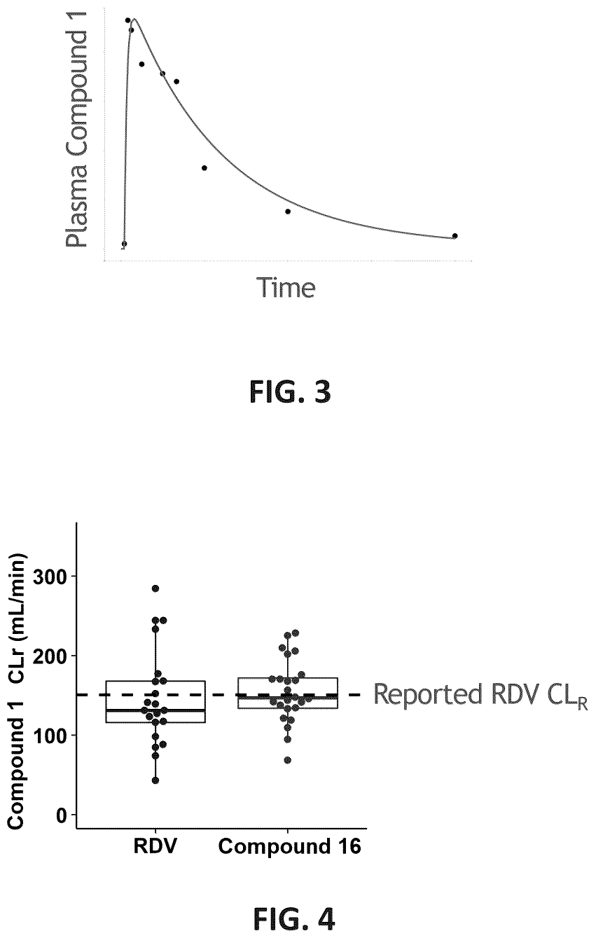

Briefly, a population-PK model was established in Monolix® (Version 2021R) using plasma data from Example 2 (Cohorts 1-3, 5 and 6). The resulting model was a 2-compartment model with first order absorption with a lag time and linear elimination. As shown in FIG. 3, the model agreed well with the compound 1 plasma concentration data from Example 2.

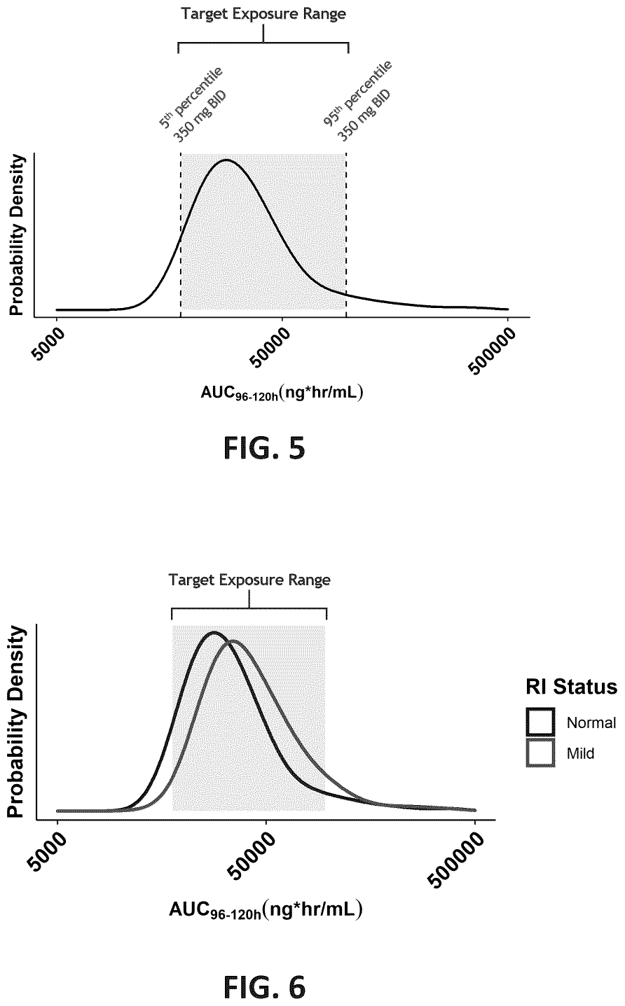

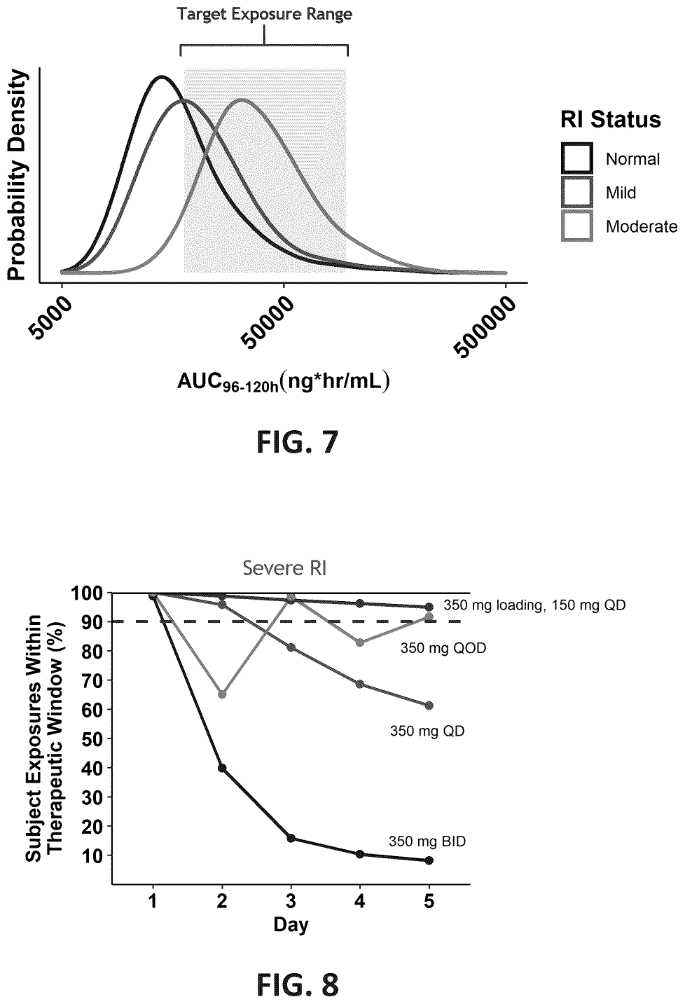

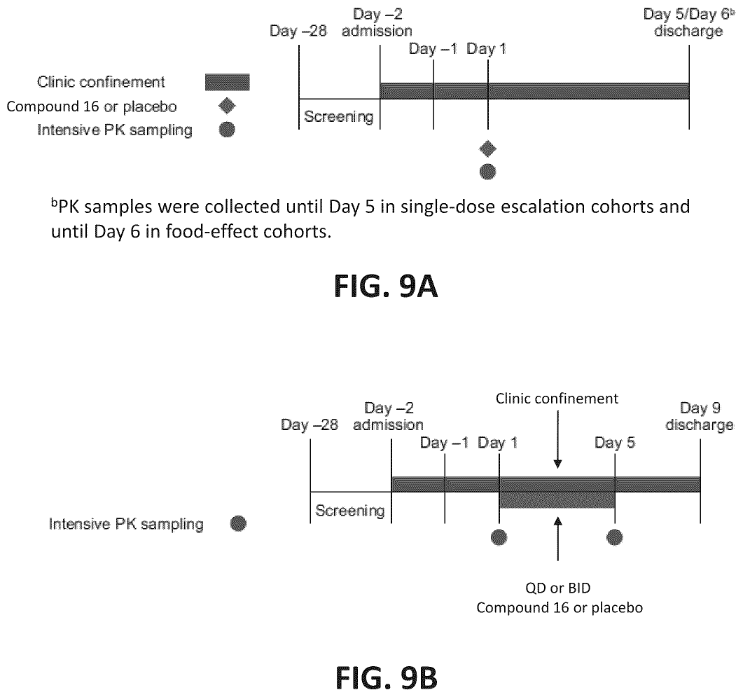

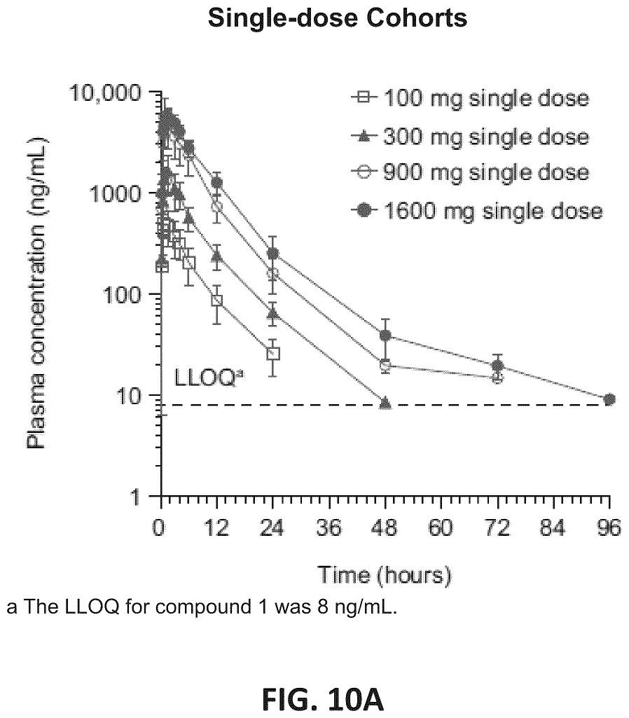

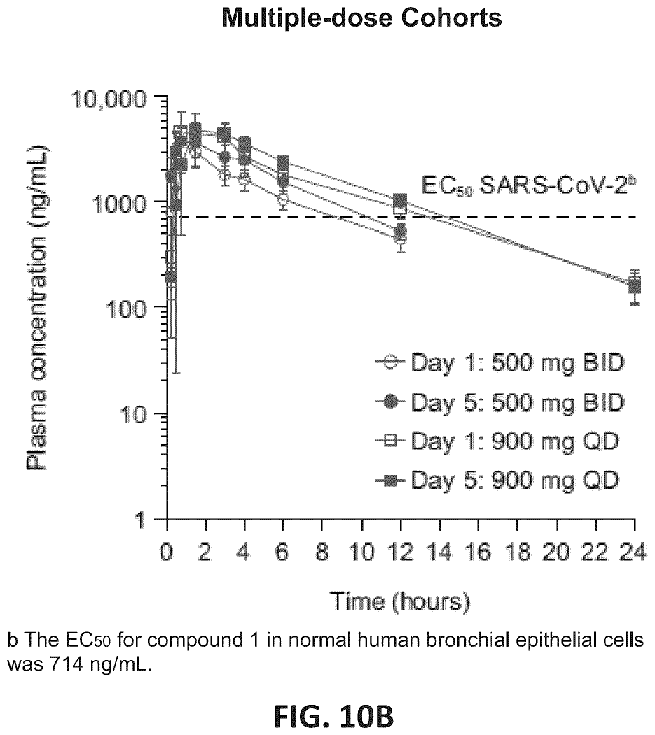

Compound 16 and remdesivir share the same renally eliminated circulating metabolite, compound 1. Observed renal clearance values (CL_{R}, the rate of excretion of the metabolite by kidney into the urine) in subjects with normal renal function (eGFR ≥90 mL/min/1.73 m², MDRD equation) from the remdesivir study and in subjects from Example 2 (Cohorts 1-4) were compared. As shown in FIG. 4, the renal clearance of compound 1 was similar between compound 16 and remdesivir (RDV), and in line with reported values for remdesivir (see, e.g., Humeniuk et al., Clin. Pharmacokinet. 60(5):569-83 (2021). Considering these similarities, the PK data obtained from the remdesivir study (Cohorts 1-4) was utilized to establish the mathematical relationship between kidney function and compound 1 elimination. Specifically, renal clearance (CL_{R}) was modeled by fitting a linear regression to eGFR (with a proportionally distributed error). CL_{R} was adjusted to bioavailability and substituted for CL of the primary compartment from the developed two compartment model. CL_{R} was assumed as the total CL for compound 1, and this model was used to simulate dosing regimens in different RI populations. Exposure targets for renal impairment population were defined as 5^{th} to 95^{th} percentile of exposures (AUC and Cₘₐₓ) expected in normal renal function (eGFR ≥90 mL/min/1.73 m²) subjects receiving 350 mg BID dose for 5 days. See FIG. 5. As shown in FIG. 6, exposures after administration of 350 mg of compound 16 BID were within the target range in >90% of subjects with mild renal impairment (eGFR 60-89 mL/min/1.73 m²). As shown in FIG. 7, exposures after administration of 350 mg of compound 16 QD were within the target range for about 90% of subjects with moderate renal impairment (eGFR 30-59 mL/min/1.73 m²). And as shown in FIG. 8, exposures after administration of a 350 mg loading dose, followed by a 150 mg QD maintenance regimen of compound 16 provided target exposures on all days in subjects with several renal impairment (eGFR 15-29 mL/min/1.73 m²). Accordingly, the following dosing regimen was proposed:
Mild Renal Impairment: 350 mg BID (twice daily) for 5 days
Moderate Renal Impairment: 350 mg QD (once daily) for 5 days
Severe Renal Impairment: 350 mg on day 1; 150 mg QD on days 2-5

### Example 4. Pharmacokinetics (PK), Safety, and Tolerability of Compound 16 in Healthy Participants

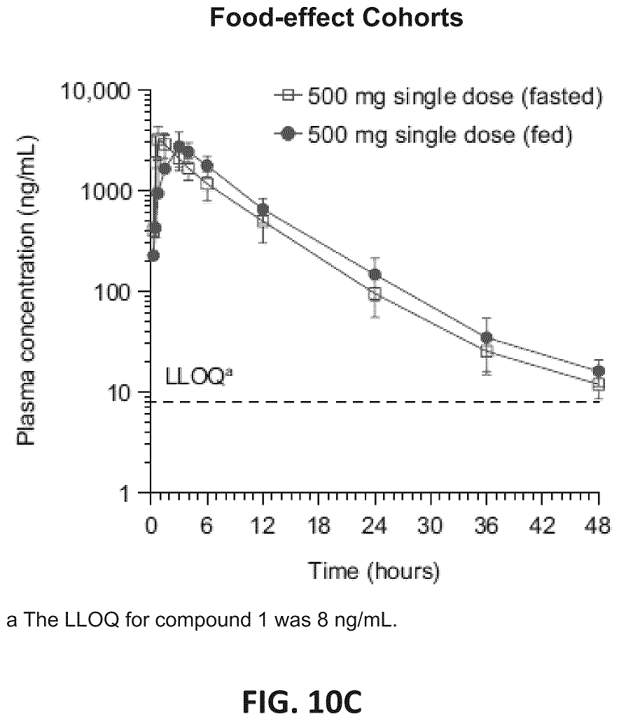

This was a randomised, blinded, placebo-controlled, Phase 1 study in which fasted healthy participants were randomly assigned (3:1) to receive compound 16 or placebo in 4 single-dose cohorts (100 mg, 300 mg, 900 mg, and 1600 mg; FIG. 9A) and 2 multiple-dose cohorts (500 mg twice daily [BID] and 900 mg once daily [QD] for 5 days; FIG. 9B). Participants in the dose-escalation cohorts (A) received the Phase 1 formulation of compound 16, whereas those in the food-effect cohorts (B) received the Phase 3 formulation of compound 16 (500 mg tablets). In the food-effect assessment (B), the no-effect bounds were defined by 2-sided 90% CIs (calculated for the ratios of GLSM of fasted vs fed cohorts) falling within [0.70 to 1.43] for AUC₀₋₂₄ and AUC_{inf} and [0.60 to 1.67] for Cₘₐₓ. The effect of food on compound 1 plasma exposures was assessed (high-fat/high-calorie breakfast, 500 mg single dose; FIG. 9A). Compound 1 plasma and urine concentrations and compound A peripheral blood mononuclear cell (PBMC) concentrations were determined using a fully validated liquid chromatography-tandem mass spectrometry method. The Phase 3 dose was selected based on the totality of the data from the first-in-human study, plasma exposures associated with efficacy in SARS-CoV-2 animal models, and prior knowledge of active metabolite generation following RDV administration.

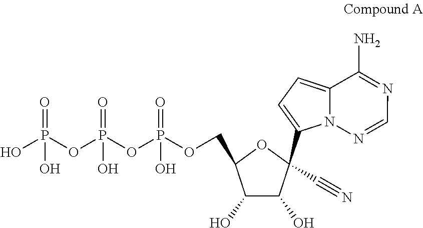 [SMILES: N#C[C@@]1(c2ccc3c(N)ncnn23)O[C@H](COP(=O)(O)OP(=O)(O)OP(=O)(O)O)[C@@H](O)[C@H]1O]

TABLE 7
Baseline Characteristics of Trial Participants

| | | | | | | | | | | Food-effect | | |
|---|---|---|---|---|---|---|---|---|---|---|---|---|
| | | Single-dose cohorts | | | | | Multiple-dose cohorts | | | cohorts | | |
| | | | | | | Pooled | 500 mg | 900 mg | Pooled | Fasted | Fed | |
| | | 100 mg | 300 mg | 900 mg | 1600 mg | placebo | BID | QD | placebo | 500 mg | 500 mg | Total |
| | | (n = 6) | (n = 6) | (n = 6) | (n = 6) | (n = 8) | (n = 6) | (n = 6) | (n = 4) | (n = 11) | (n = 11) | (n = 70) |
| Age, mean | | 32 | 30 | 35 | 34 | 32 | 33 | 32 | 29 | 33 | 33 | 32 |
| (SD; years) | | (9.7) | (8.7) | (6.0) | (7.5) | (8.5) | (7.7) | (7.7) | (5.3) | (8.9) | (6.9) | (7.5) |
| Sex, n | Female | 2 | 1 | 3 | 2 | 5 | 2 | 4 | 2 | 6 | 6 | 33 |
| (%) | | (33.3) | (16.7) | (50.0) | (33.3) | (62.5) | (33.3) | (66.7) | (50.0) | (54.5) | (54.5) | (47.1) |
| Race, n | American | 0 | 0 | 0 | 0 | 1 | 0 | 1 | 0 | 0 | 0 | 2 |
| (%) | Indian or | | | | | (12.5) | | (16.7) | | | | (2.9) |
| | Alaska | | | | | | | | | | | |
| | Native | | | | | | | | | | | |
| | Asian | 0 | 1 | 0 | 0 | 0 | 0 | 0 | 1 | 2 | 2 | 6 |
| | | | (16.7) | | | | | | (25.0) | (18.2) | (18.2) | (8.6) |
| | Black | 1 | 3 | 2 | 5 | 4 | 2 | 2 | 1 | 7 | 4 | 31 |
| | | (16.7) | (50.0) | (33.3) | (83.3) | (50.0) | (33.3) | (33.3) | (25.0) | (63.6) | (36.4) | (44.3) |
| | White | 5 | 2 | 4 | 1 | 2 | 4 | 3 | 1 | 2 | 5 | 29 |
| | | (83.3) | (33.3) | (66.7) | (16.7) | (25.0) | (66.7) | (50.0) | (25.0) | (18.2) | (45.5) | (41.4) |
| | Other | 0 | 0 | 0 | 0 | 1 | 0 | 0 | 1 | 0 | 0 | 2 |
| | | | | | | (12.5) | | | (25.0) | | | (2.9) |

| | | | | | | | | | | | |
|---|---|---|---|---|---|---|---|---|---|---|---|
| Weight, mean | 75.7 | 73.9 | 75.9 | 79.6 | 73.1 | 77.3 | 73.2 | 80.9 | 74.0 | 72.2 | 75.0 |
| (SD; kg) | (11.6) | (13.6) | (12.1) | (14.1) | (13.6) | (12.0) | (11.1) | (8.3) | (11.1) | (14.0) | (11.9) |

BID, twice daily;
QD, once daily;
SD, standard deviation

Plasma PK

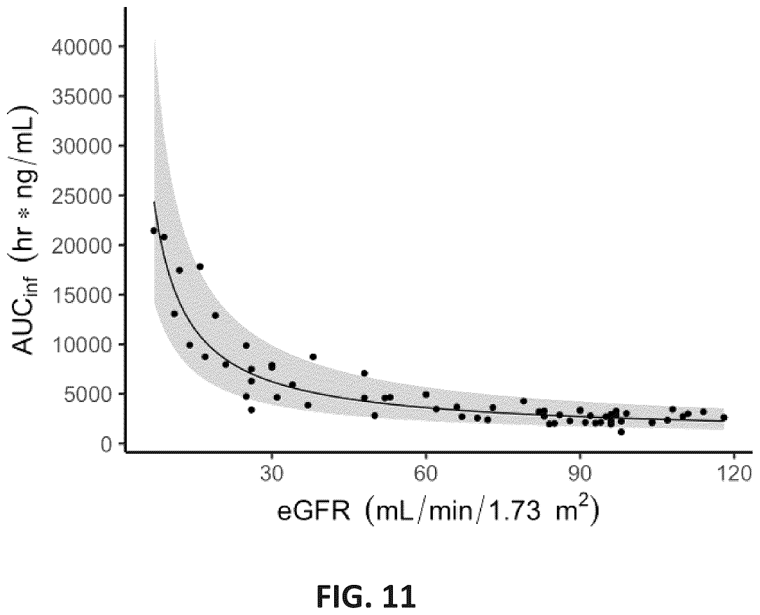

Predictable and consistent levels of the compound I metabolite were observed in plasma shortly (˜0.75 hours) after administration; compound 16 prodrug levels were either undetectable or low and transient (FIGS. 10A, 10B, and 10C). Compound 1 exhibited linear and dose-proportional PK in the 100 to 900 mg range; less than dose-proportional increases were observed at the 1600 mg dose; terminal plasma elimination half-life was 6 to 7 hours across the 100 to 900 mg single-dose cohorts. Multiple-dose PK were consistent with single-dose PK (Table 8). Accumulation was ˜12% after QD dosing and ˜35% after BID dosing; steady state was reached on Day 4 of dosing.

TABLE 8
Mean (% CV) Compound 1 Plasma PK Parameters in Healthy
Participants Following a Single Dose of Compound 16

| | | | | | Multiple-dose cohorts | | | |
|---|---|---|---|---|---|---|---|---|
| | | | | | Day 1: | Day 5: | Day 1: | Day 5: |
| | Single-dose cohorts | | | | 500 mg | 500 mg | 900 mg | 900 mg |
| PK | 100 mg | 300 mg | 900 mg | 1600 mg | BID | BID | QD | QD |
| parameter^{a} | (n = 6) | (n = 6) | (n = 6) | (n = 6) | (n = 6) | (n = 6) | (n = 6) | (n = 6) |
| Cₘₐₓ | 570 | 1830 | 5940 | 7170 | 3820 | 4620 | 6230 | 5180 |
| (ng/mL) | (30.5) | (32.6) | (44.6) | (26.7) | (32.7) | (18.2) | (14.5) | (19.6) |
| Tₘₐₓ (h) | 0.75 | 0.78 | 0.75 | 1.5 | 0.75 | 0.75 | 0.75 | 1.5 |
| | (0.50- | (0.50- | (0.75- | (0.75- | (0.75- | (0.50- | (0.75- | (1.5- |
| | 0.75) | 1.50) | 1.50) | 1.53) | 1.50) | 1.50) | 1.50) | 3.0) |
| t_{1/2} (h) | 5.85 | 6.1 | 7.36 | 15.7 | — | — | — | — |
| | (5.57- | (4.86- | (6.89- | (14.1- | | | | |
| | 6.26) | 6.93) | 7.81) | 17.6) | | | | |
| AUC | 3700 | 10,400 | 34,600 | 48,300 | 15,900 | 21,500 | 32,000 | 35,700 |
| (h · ng/mL)^{b} | (34.7) | (21.4) | (34.7) | (22.4) | (18.2) | (19.3) | (11.6) | (11.6) |
| AUC₀₋₂₄ | — | — | — | — | ~31,800^{c} | ~43,000^{c} | 32,000 | 35,700 |
| (h · ng/mL) | | | | | | | (11.6) | (11.6) |

% CV, percentage coefficient of variation; PK, pharmacokinetic; BID, twice daily; QD, once daily; Cₘₐₓ, maximum observed concentration; Tₘₐₓ, time to maximum observed concentration; t_{1/2}, terminal elimination half-life; AUC, area under the concentration-time curve; AUC₀₋₂₄, area under the concentration-time curve from 0 to 24 hours; Q1, Quartile 1; Q3, Quartile 3; AUC_{inf}, area under the concentration-time curve extrapolated to infinite time; AUCD1, area under the concentration-time curve on Day 1 over the dosing interval; AUCₜₐᵤ, area under the concentration-time curve at steady state over the dosing interval; AUC₀₋₁₂, area under the concentration-time curve from 0 to 12 hours.
^{a}Data are presented as mean (% CV), with the exception of Tₘₐₓ and t_{1/2}, which are presented as median (Q1-Q3).
^{b}AUC_{inf} is reported for single-dose cohorts; AUC_{D 1} is reported for Day 1, and AUCₜₐᵤ is reported at steady state for Day 5 in multiple-dose cohorts.
^{c}BID dosing AUC₀₋₂₄ was calculated as 2 × AUC₀₋₁₂ and does not account for the expected additional accumulation within Day 1; Day 1 AUC₀₋₁₂ = 15,900 (18.2); Day 5 AUC₀₋₁₂ = 21,500 (19.3).

Intracellular PK

Dose-proportional increases in the intracellular concentrations of compound A were observed in PBMCs; substantial accumulation was observed after repeat dosing (6-fold for BID and 3-fold for QD). At the selected Phase 3 dosing regimen of 350 mg BID for 5 days, exposures of the active metabolite (compound A) are likely to exceed those observed with the approved IV RDV dosing regimen (200 mg loading dose followed by 100 mg QD). Levels of compound I in plasma needed to produce the active triphosphate metabolite inside cells are ˜14-fold higher after compound 16 than after RDV.

Table 8A presents a comparison of steady-state pharmacokinetics (PK) of metabolites (GS-443902 and GS-441524) following administration of the Phase 3 dosing regimens for oral compound 16 (350 mg twice daily) and intravenous (IV) RDV (200/100 mg once daily).

TABLE 8A
Comparison of Steady-State PK of Metabolites
Following Administration of Oral Compound 16 and IV RDV

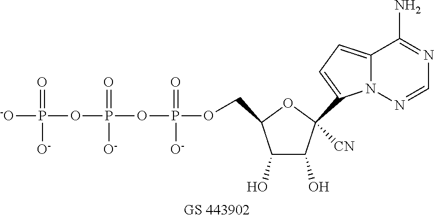

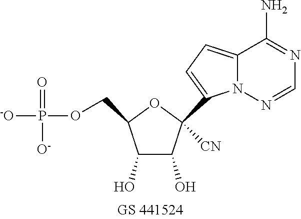

| | | |
|---|---|---|
| | Compound 16^{b} | RDV^{c} |
| | Phase 3 Regimen | Approved Regimen |
| Metabolite | 350 mg BID | 200/100 mg QD |
| PK Parameterª | (N = 6) | (N = 26) |

GS-443902 in PBMCs

| | | |
|---|---|---|
| AUC₀₋₂₄, h * μM^{d} | 591 (49.1) | 240 (25.4) |
| Ctan, μM | 32.2 (33.4) | 10.2 (49.5) |

GS-441524 in plasma

| | | |
|---|---|---|
| AUC₀₋₂₄, h * ng/mL | 30,100 (19.3) | 2230 (18.4) |
| Cₘₐₓ, ng/ml | 3240 (18.2) | 145 (19.3) |

%CV = percentage coefficient of variation; BID = twice daily; IV = intravenous; Compound 16 = obeldesivir (ODV; GS-5245); PBMC = peripheral blood mononuclear cell; PK = pharmacokinetic(s); QD = once daily; RDV = remdesivir
^{a}Data presented as mean (% CV).
^{b}Reference: GS-US-611-6248 multiple-dose PK parameters following 500 mg BID oral administration for 5 days of ODV to healthy participants (Cohort 5) were scaled to 350 mg BID, as supported by dose proportionality in this range.
^{c}Reference: Humeniuk R, et al. Clinical Pharmacokinet 2021;60:569-583; GS-US-399-5505, multiple-dose PK parameters following IV administration of RDV 100 mg for 5-10 days to healthy participants.
^{d}AUC₀₋₂₄ calculated as 2 × AUCₜₐₙ for ODV.

Urine PK

Approximately 40% to 45% of the compound 16 dose was recovered as compound 1 in urine over the first 24 hours postdose. Compound 16 was not detectable in urine. Compound 1 metabolite renal clearance was approximately 150 to 180 mL/min across all single-dose cohorts. These values are consistent with historical data describing the urine PK of compound 1 after IV RDV.

Food-Effect Study

Administration of compound 16 with a high-fat meal decreased the rate (time to maximum observed concentration increased from 0.75 to 3.0 hours) but not the extent of absorption; therefore, compound 16 can be given without regard to food (Table 9).

TABLE 9
Compound 1 Plasma PK Parameters in Fasted Versus Fed Healthy
Participants Receiving a Single Dose of Compound 16

| | Fasted | Fed | |
|---|---|---|---|
| | 500 mg | 500 mg | GLSM |
| PK parameter^{a} | (n = 11) | (n = 11) | (90% CI) |
| Cmax (ng/mL) | 3600 | 3430 | 0.941 |
| | (33.3) | (35.3) | (0.73-1.21) |
| Tₘₐₓ (h) | 0.75 | 3.0 | — |
| | (0.75-1.50) | (3.0-4.0) | |
| AUC₀₋₂₄ (h · ng/mL) | 20,903 | 23,204 | 1.122 |
| | (18.7) | (13.5) | (0.98-1.28) |
| AUCₗₐₛₜ (h · ng/mL) | 22,000 | 24,500 | 1.129 |
| | (18.4) | (13.2) | (0.99-1.29) |
| AUC_{inf} (h · ng/mL) | 22,100 | 24,700 | 1.129 |
| | (18.3) | (13.2) | (0.99-1.29) |
| AUCₑₓₜᵣₐₚ (%) | 0.52 | 0.55 | — |
| | (33.8) | (40.8) | |
| t_{1/2} (h) | 6.3 | 6.2 | — |
| | (5.5-6.6) | (5.0-6.6) | |

PK, pharmacokinetic; GLSM, geometric least-squares mean; CI, confidence interval; Cₘₐₓ, maximum observed concentration; Tₘₐₓ, time to maximum observed concentration; AUC₀₋₂₄, area under the concentration-time curve from 0 to 24 hours; AUCₗₐₛₜ, area under the concentration-time curve from dosing to last measurable concentration; AUC_{inf}, area under the concentration-time curve extrapolated to infinite time; AUCₑₓₜᵣₐₚ, area under the concentration-time curve extrapolated as a percentage of the total; t_{1/2}, terminal elimination half-life; % CV, percentage coefficient of variation; Q1, Quartile 1; Q3, Quartile 3.
^{a}Data are presented as mean (% CV), with the exception of Tₘₐₓ and t_{1/2}, which are presented as median (Q1-Q3).

Safety

Safety data are summarised below (Table 10). Of the 14 AEs reported, 13 were Grade 1 and one was Grade 2. There were 3 study drug-related AEs, all of which were headaches: 2 of 58 (3.4%) in participants who received compound 16 and 1 of 12 (8.3%) in the pooled placebo group. The most common laboratory abnormality was a decrease in creatinine clearance (Table 11). Overall, 18 of 58 (31%) participants who received compound 16 experienced a decrease in creatinine clearance compared to 3 of 12 (25%) participants in the pooled placebo group. All creatinine clearance decreases in the fasted and fed cohorts occurred on Days 5 or 6.

TABLE 10
Summary of AEs

| | | | | | | | | | Food-effect | | |
|---|---|---|---|---|---|---|---|---|---|---|---|
| | Single-dose cohorts | | | | | Multiple-dose cohorts | | | cohorts | | |
| | | | | | Pooled | 500 mg | 900 mg | Pooled | Fasted | Fed | |
| | 100 mg | 300 mg | 900 mg | 1600 mg | placebo | BID | QD | placebo | 500 mg | 500 mg | Total |
| n (%) | (n = 6) | (n = 6) | (n = 6) | (n = 6) | (n = 8) | (n = 6) | (n = 6) | (n = 4) | (n = 11) | (n = 11) | (n = 70) |
| AE | 1 | 0 | 0 | 2 | 2 | 2 | 1 | 2 | 3 | 1 | 14 |
| | (16.7) | | | (33.3) | (25.0) | (33.3) | (16.7) | (50.0) | (27.3) | (9.1) | (20.0) |
| Grade ≥3 AE | 0 | 0 | 0 | 0 | 0 | 0 | 0 | 0 | 0 | 0 | 0 |
| AE related to | 0 | 0 | 0 | 0 | 0 | 1 | 0 | 1 | 1 | 0 | 3 |
| study drug | | | | | | (16.7) | | (25.0) | (9.1) | | (4.3) |

AE, adverse event; BID, twice daily; QD, once daily.

TABLE 11
Summary of Laboratory Abnormalities^{a}

| | | | | | | | | | Food-effect | | |
|---|---|---|---|---|---|---|---|---|---|---|---|
| | Single-dose cohorts | | | | | Multiple-dose cohorts | | | cohorts | | |
| | | | | | Pooled | 500 mg | 900 mg | Pooled | Fasted | Fed | |
| | 100 mg | 300 mg | 900 mg | 1600 mg | placebo | BID | QD | placebo | 500 mg | 500 mg | Total |
| n (%) | (n = 6) | (n = 6) | (n = 6) | (n = 6) | (n = 8) | (n = 6) | (n = 6) | (n = 4) | (n = 11) | (n = 11) | (n = 70) |
| Grade 2 | 1 | 1 | 2 | 6 | 2 | 0 | 2 | 1 | 2 | 5 | 22 |
| | (16.7) | (16.7) | (33.3) | (100) | (25.0) | | (33.3) | (25.0) | (18.2) | (45.5) | (31.4) |
| Creatinine | 0 | 1 | 2 | 6 | 2 | 0 | 2 | 1 | 2 | 5 | 21 |
| clearance | | (16.7) | (33.3) | (100) | (25.0) | | (33.3) | (25.0) | (18.2) | (45.5) | (30.0) |
| decrease^{b} | | | | | | | | | | | |
| Grade 3 | 0 | 1 | 0 | 0 | 0 | 0 | 0 | 0 | 0 | 0 | 1 |
| | | (16.7) | | | | | | | | | (1.4) |
| Lipase | 0 | 1 | 0 | 0 | 0 | 0 | 0 | 0 | 0 | 0 | 1 |
| increase | | (16.7) | | | | | | | | | (1.4) |

BID, twice daily; QD, once daily.
^{a}Severity grades were defined by the Division of AIDS (DAIDS) Table for Grading the Severity of Adult and Pediatric Adverse Events (Version 2.1).
^{b}All Grade 2 creatinine clearance abnormalities correspond to only one Grade 1 laboratory abnormality in creatinine in the 1600 mg single-dose cohort.

Oral administration of compound 16 resulted in predictable and consistent plasma exposures of the nucleoside compound 1. Target therapeutic exposures of plasma compound 1 and the active intracellular metabolite compound A were reached or exceeded at doses of 900 mg QD and 500 mg BID for 5 days, respectively. Administration of compound 16 was safe and well tolerated.

### Example 5: Dose Optimization of Compound 16 in Subjects with Renal Impairment

A population-PK model was developed using nonlinear mixed effects modeling and utilizing plasma concentration data of compound 1 from GS-US-611-6248 Study shown in Example 4. Plasma concentration data from phase 1 study of RDV in non-COVID-19 participants (GS-US-540-9015) with renal impairment (RI) was used to characterize the relation between eGFR and renal clearance (CL_{R}) of compound 1. This relation between eGFR and CL_{R} was incorporated to the Compound 16 PopPK model. Based on this study, CL_{R} is assumed as the total CL for compound 1, and this model was used to simulate dosing regimens in population with RI. Dose adjustment needed for those with decreased renal function were simulated to target the 5^{th} to 95^{th} percentile of simulated plasma exposures (AUC) in those with normal renal function receiving 350 mg BID dose for 5 days (Phase 3 dosing regimen) or 95m percentile of 500 mg BID in normal renal function, which was well tolerated in GS-US-611-6248 Study.

A 2-compartment model with transit compartment absorption and linear elimination adequately described the compound 1 plasma concentration data following PO of compound 16. The quadratic relationship between compound 1 CL_{R} and eGFR, established based on data collected in study with RDV, was adjusted for oral bioavailability of compound 16 and substituted for CL of the primary compartment in the compound 16 Pop-PK model. Based on the simulations, 350 mg BID, 350 mg QD, and 350 mg on Day 1 followed by 175 mg QD on Days 2-5 were projected for participants with mild, moderate and severe RI, respectively.

### Example 6: Compound 16 Dosing Regimen for COVID-19 Patients with Preexisting Renal Impairment (RI)

The PK of compound 1 in plasma and urine following oral administration of compound 16 were extensively characterized in a dedicated Phase 1 dose-ranging study in healthy volunteers with normal renal function (Study GS-US-611-6248). The same circulating compound 1 metabolite could be detected following intravenous (IV) remdesivir administration in healthy volunteers but at lower levels than after compound 16 at clinically relevant doses (Study GS-US-399-5505). Data from these studies indicate approximately 14-fold lower compound 1 steady-state exposure (i.e., AUC) in participants with normal renal function after 100 mg IV remdesivir relative to the projected therapeutic target exposures after oral administration of compound 16 350 mg BID for 5 days (see Table 12).

TABLE 12
Comparison of steady-state compound 1 plasma
PK following IV administration of remdesivir
and oral administration of compound 16

| | Compound 16 | Remdesivir Steady |
|---|---|---|
| | Steady State | State Approved |
| | 350 mg BID | Regimen COVID-19 |
| | Phase 3 Regimen | (200/100 mg) |
| PK parameter^{a} | (N = 6)^{c,} ^{d} | (N = 26)^{e} |
| AUC_{0-24 h} (h*ng/ml)^{b} | 30,100 | 2,230 |
| | (19.3) | (18.4) |
| Cₘₐₓ (ng/mL) | 3236 | 145 |
| | (18.2) | (19.3) |
| Tₘₐₓ (h) | 0.75 | 1.5 |
| | (0.50, 1.50) | (1.5, 2.0) |
| T_{1/2} (h) | 6.1 | 27.36 |
| | (5.0, 6.9) | (25.30, 30.3) |

BID = twice daily
^{a}AUC and Cₘₐₓ are presented as mean (% CV); Tₘₐₓ and t_{1/2} are presented as median (Q1, Q3)
^{b}AUC_{0-24 h} calculated as 2xAUC_{0-12 h} for compound 16
^{c}Reference: Phase 1 Study GS-US-611-6248; multiple dose PK parameters following 500 mg BID oral administration of compound 16 (Cohort 5, N = 6) were scaled to 350 mg BID, as supported by dose proportionality in this range
^{d}Reference: Phase 1 Study GS-US-611-6248; single dose PK parameters following oral administration of compound 16 300 mg (Cohort 2, N = 6)
^{e}Reference: Phase 1 Study GS-US-399-5505; multiple dose PK parameters following intravenous administration of remdesivir 100 mg

Despite the different plasma PK profiles of compound 1, renal clearance (CLr) after remdesivir or compound 16 administration was comparable. Compound 1 CLr was calculated by measuring the amount of compound 1 excreted in urine following drug administration over the corresponding plasma AUC. Since compound 1 CLr is similar for both compounds, it is appropriate to use CLr of compound 1 from the remdesivir program to model dose projections for compound 16.

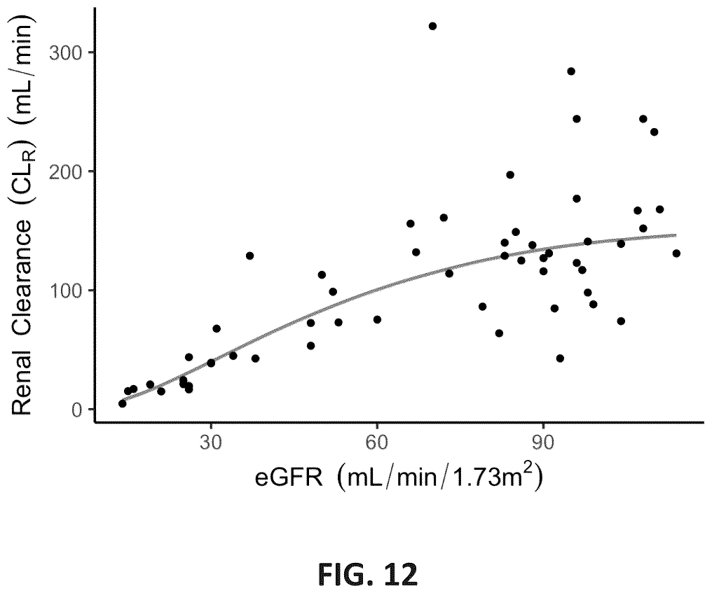

The relationship between plasma and urine PK of compound 1 and CLr has been fully characterized in the dedicated Phase 1 study for remdesivir (Study GS-US-540-9015), which enrolled participants with mild, moderate, severe RI, and kidney failure (see Table 13 and FIG. 11). In this study, moderate and severe RI resulted in an approximately 2.02 and 3.26-fold increase in plasma compound 1 AUC_{inf} and a 1.44 and 1.68-fold increase in Cm. The increases in plasma levels of compound 1 were closely correlated with decreases in CLr, as measured by urinary output.

The relationship between plasma exposures of compound 1 and renal function was explored by linear regression analysis of AUC_{inf} versus eGFR_{MDRD} following remdesivir administration. A strong correlation of plasma exposure to eGFR (see FIG. 11) is consistent with the predominant renal elimination pathway for compound 1. To note, eGFR was estimated using Modification of Diet in Renal Disease (MDRD) equation shown below. Furthermore, participants with kidney failure who were on dialysis are not included in this regression analysis.

$$log ( {AUC}_{{dc}_{i}} ) = {β}_{0} + {β}_{1} * log ( \frac{e G R {F}_{i}}{90} ) + {β}_{2} * {( log ( \frac{e G R {F}_{1}}{90} ) )}^{2} + {ε}_{i}$$

$${ε}_{i} ∼ N ( 0 , {δ}^{2} )$$

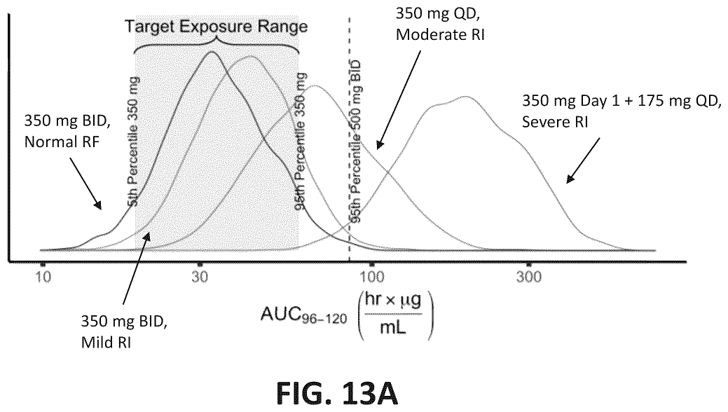

A similar quadratic relationship was established between eGFR and CLr of compound 1 utilizing these data from IV remdesivir administration estimated using the below equation (see FIG. 12).

$$log ( {CL}_{i} ) = {β}_{0} + {β}_{1} * log ( \frac{e G R {F}_{i}}{90} ) + {β}_{2} * {( log ( \frac{e G R {F}_{1}}{90} ) )}^{2} + {ε}_{i}$$

$${ε}_{i} ∼ N ( 0 , {δ}^{2} )$$

TABLE 13
Effect of renal impairment on plasma PK and estimated renal clearance (CLr) of
compound 1 following a single IV administration of remdesivir (GS-US-540-9015)

| | | | | Kidney Failure | | |
|---|---|---|---|---|---|---|
| | Mild RI | Moderate RI | Severe RI | (eGFR <15 mL/min/1.73 m²) | | |
| GLSM | (eGFR 60-89 | (eGFR 30-59 | (eGFR 15-29 | remdesivir | remdesivir | No |
| Ratio | mL/min/1.73 m²) | mL/min/1.73 m²) | mL/min/1.73 m²) | Pre-HD | Post-HD | Dialysis |
| (90% CI) | (N = 10) | (N = 10) | (N = 10) | (N = 6) | (N = 6) | (N = 3) |
| AUC_{inf}^{a} | 119 | 202 | 326 | 497 | 622 | 787 |
| | (96.7, 147) | (157, 262) | (239, 446) | (365, 677) | (444, 871) | (649, 953) |
| Cₘₐₓ | 107 | 144 | 168 | 227 | 307 | 300 |
| | (90.1, 126) | (113, 185) | (128, 220) | (172, 299) | (221, 426) | (263, 342) |
| CLr | 144.3 | 70.0 | 19.9 | — | — | 16.0 |
| (mL/min)^{a} | (50.3) | (47.1) | (50.0) | | | (41.2) |
| | 159.1 | 125.2 | 136.0 | | | 193.1 |
| | (56.0) | (23.0) | (35.1) | | | (60.1) |

GLSM = Geometric least squares means; HD = Hemodialysis.
^{a}Renal clearance is reported as mean (% CV). The top values present the estimated renal clearance in participants with RI, and the bottom values show the estimates for the healthy matched controls of each group.

The findings from RI remdesivir (GS-US-540-9015) and FIH compound 16 (GS-US-611-6248) studies were used to evaluate the potential exposure of compound 1 in subjects with varying degrees of RI. The population-PK model described in Example 3 was further refined using nonlinear mixed effects modeling and plasma concentration of compound 1 after oral administration of compound 16 to healthy participants from the FIH study (GS-US-611-6248) at doses ranging from 100-900 mg. Cohort 4 (1600 mg) was excluded due to potential nonlinearity in the PK and not being a clinically relevant dose. A 2-compartment model with transit compartment absorption and linear elimination adequately described the compound 1 plasma concentration data following oral administration of compound 16. In order to add eGFR as a covariate to this model, the quadratic relationship established between compound 1 CLr and eGFR (see FIG. 12) based on data from the RI remdesivir study (GS-US-540-9015) was used. In this PK model, the total clearance of compound I after compound 16 administration was assumed to be equal to the CLr (a reasonable assumption considering compound 1 is predominantly renally eliminated). The developed model was used to simulate plasma PK profiles following various potential dosing regimens of compound 16 in different RI populations.

Compound 1 plasma exposure (AUC) targets for the participants with varying degrees of RI receiving compound 16 were established as the 5^{th} to 95^{th} percentile of simulated compound 1 exposures in participants with normal renal function and those with mild RI receiving 350 mg BID doses for 5 days (Phase 3 dosing regimen) and below the 95^{th} percentile of simulated exposures following 500 mg BID (the highest multiple ascending dose exposures evaluated and shown to be safe in the FIH compound 16 study).

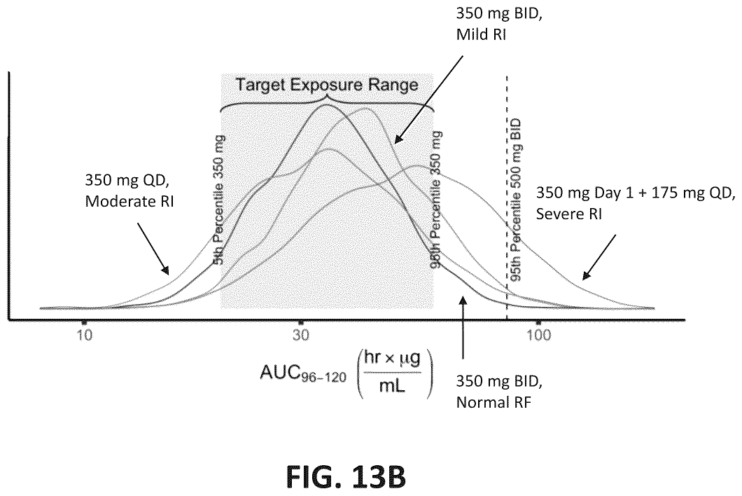

Based on the simulations, compound 16 dose adjustment is not required for subjects with mild RI, or those with eGFR ≥60 mL/min, as the predicted exposures of compound 1 are anticipated to be comparable to those with normal renal function (see FIG. 13A, FIG. 13B, and Table 14), which is within the target therapeutic exposure range. For subjects with moderate RI (30≤eGFR<60 mL/min), a 350 mg once-daily (QD) regimen (see FIG. 13A and FIG. 13B) is proposed, as the distribution of predicted compound 1 exposures are mostly within the target therapeutic exposure range, and the majority of participants are below the 95^{th} percentile of predicted exposures following 500 mg BID dose shown to be safe in the FIH compound 16 study (see Table 15).

For severe RI (15≤eGFR<30 mL/min), simulations suggest that a 350 mg dose of compound 16 on Day 1 followed by 175 mg daily doses (half the 350 mg strength tablet) on Days 2-5 provide plasma exposures of compound 1 (predicted median AUC₀₋₂₄ₕ) of 42.8 h·μg/mL with a 90% prediction interval (5^{th} to 95^{th} percentiles) of 24.2 to 72.0 h·μg/mL (see Table 14). These predicted plasma exposures in the severe RI population are higher than those with normal renal function receiving 350 mg BID but still ensure that the majority of the participants with severe RI (˜89%) have plasma AUC below the 500 mg BID dose shown to be safe in the FIH compound 16 study (see Table 15).

The selected dosing regimen for the moderate renal impairment population allows utilization of the available manufactured dosage form of 350 mg tablets with a simple/easy-to-follow reduction in frequency. Similarly, for participants with severe RI decreasing the dose on Days 2-5 by half (175 mg) and the frequency to once daily is equivalent to a 4-fold dose reduction which should be easy to follow in the clinical practice.

TABLE 14
Model Predicted Exposures of compound 1 Following Different
Dosing Regimens of compound 16 in Participants with
Normal, Mild, Moderate, and Severe Renal Impairment

| | | Day 1 [AUC₀₋₂₄ | Day 5 [AUC₉₆₋₁₂₀ |
|---|---|---|---|
| Dosing Regimen | Renal Function | (h · μg/mL)] | (h · μg/mL)] |
| 500 mg BID | eGFR ≥ | 41.7 | 47.9 |
| (Days 1-5)^{a} | 90 mL/min | (25.2, 71.5) | (28.7, 85.2) |
| 350 mg BID | eGFR ≥ | 31.3 | 36.9 |
| (Days 1-5)^{b} | 60 mL/min | (18.1, 52.8) | (20.8, 64.7) |
| 350 mg QD | 30 ≤ eGFR < | 26.4 | 34.1 |
| (Days 1-5)^{c} | 60 mL/min | (14.5, 45.8) | (17.0, 65.8) |
| 350 mg (Day 1) + | 15 ≤ eGFR < | 42.8 | 49.8 |
| 175 mg QD | 30 mL/min | (24.2, 72.0) | (24.1, 100.6) |
| (Days 2-5)^{d} | | | |

BID = twice daily; eGFR = estimated glomerular filtration rate; FIH = first in human; QD = once daily
eGFR ≥ 90 mL/min represents participants with normal renal function
eGFR ≥ 60 mL/min represents participants with normal renal function and those with mild renal impairment
30 ≤ eGFR < 60 mL/min represents participants with moderate renal impairment.
15 ≤ eGFR < 30 mL/min represents participants with severe renal impairment.
AUC estimates are presented as median (90% prediction interval).
^{a}FIH dosing regimen
^{b}Phase 3 dosing regimen for participants with normal renal function and those with mild renal impairment
^{c}Phase 3 dosing regimen for participants with moderate renal impairment
^{d}Proposed dosing regimen for participants with severe renal impairment

TABLE 15
Predicted Exposures in Participants with Moderate or Severe
Renal Impairment Following the Proposed Dosing Regimens

| | % Within 350 mg BID | % Below 95^{th} |
|---|---|---|
| | AUC^{a} | Percentile of |
| | (5^{th}-95^{th} | 500 mg |
| RI Status/Regimen | Percentile) | BID AUC^{b} |
| Moderate RI | 80.7 | 98.6 |
| (30 ≤ eGFR < 60 mL/min)/ | | |
| 350 mg QD (Days 1-5) | | |
| Severe RI | 62.2 | 88.4 |
| (15 ≤ eGFR < 30 mL/min)/ | | |
| 350 mg (Day 1) + 175 mg QD | | |
| (Days 2-5) | | |

BID = twice daily; QD = once daily
^{a}Predicted exposures (AUC₉₆₋₁₂₀) in participants with normal renal function or mild renal impairment (eGFR ≥ 60 mL/min).
^{b}Predicted exposures (AUC₉₆₋₁₂₀) in participants with normal renal function.

All references, including publications, patents, and patent documents are incorporated by reference herein, as though individually incorporated by reference. The present disclosure provides reference to various embodiments and techniques. However, it should be understood that many variations and modifications may be made while remaining within the spirit and scope of the present disclosure. The description is made with the understanding that it is to be considered an exemplification of the claimed subject matter and is not intended to limit the appended claims to the specific embodiments illustrated.

1. A method of treating a viral infection in a patient in need thereof, wherein the method comprises administering to the patient a compound 16: 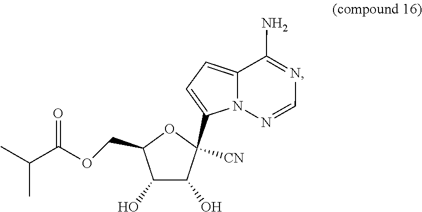 [SMILES: CC(C)C(=O)OC[C@H]1O[C@@](C#N)(c2ccc3c(N)ncnn23)[C@H](O)[C@@H]1O]
a deuterated compound thereof, or a pharmaceutically acceptable salt thereof;
wherein the compound 16, the deuterated compound thereof, or the pharmaceutically acceptable salt thereof, is administered in a dosage of less than 1,600 mg/dose.

2.-7. (canceled)

8. The method of claim 1, wherein the patient has an eGFR of 60 mL/min/1.73 m² to 89 mL/min/1.73 m².

9. The method of claim 8, wherein the patient has mild renal impairment.

10.-13. (canceled)

14. The method of claim 8, wherein the compound 16, the deuterated compound thereof, or the pharmaceutically acceptable salt thereof, is administered twice daily.

15. (canceled)

16. (canceled)

17. The method of claim 8, wherein the compound 16, the deuterated compound thereof, or the pharmaceutically acceptable salt thereof, is administered in a dosage of 250 mg/dose to 500 mg/dose.

18. The method of claim 8, wherein the compound 16, the deuterated compound thereof, or the pharmaceutically acceptable salt thereof, is administered in a dosage of 350 mg/dose.

19. (canceled)

20. (canceled)

21. The method of claim 1, wherein the administration results in a mean Cₘₐₓ of less than 7,000 ng/mL of a compound 1: 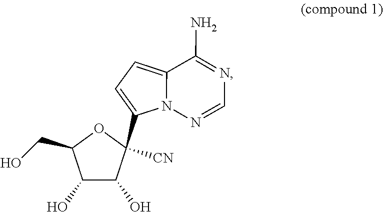 [SMILES: N#C[C@@]1(c2ccc3c(N)ncnn23)O[C@H](CO)[C@@H](O)[C@H]1O]
or a deuterated compound thereof.

22.-24. (canceled)

25. The method of claim 1, wherein the administration results in a mean AUC₀₋₂₄ of less than 44,000 ng/mL*h of a compound 1: 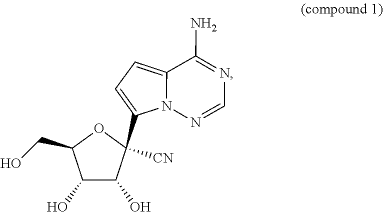 [SMILES: N#C[C@@]1(c2ccc3c(N)ncnn23)O[C@H](CO)[C@@H](O)[C@H]1O]
or a deuterated compound thereof.

26.-28. (canceled)

29. The method of claim 1, wherein
the patient has normal renal function or mild renal impairment; and
the compound 16, the deuterated compound thereof, or the pharmaceutically acceptable salt thereof, is administered twice daily in a dosage of 350 mg/dose.

30. The method of claim 1, wherein
the patient has normal renal function or mild renal impairment;
the compound 16, the deuterated compound thereof, or the pharmaceutically acceptable salt thereof, is administered twice daily in a dosage of 350 mg/dose; and
the administration results in a mean Cₘₐₓ of 2,000 ng/mL to 6,200 ng/mL and a mean AUC₀₋₂₄ of 18,200 ng/mL*h to 36,400 ng/mL*h of a compound 1: 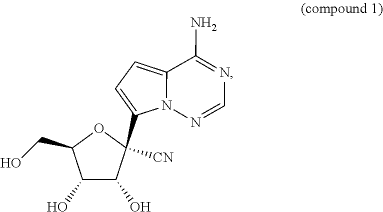 [SMILES: N#C[C@@]1(c2ccc3c(N)ncnn23)O[C@H](CO)[C@@H](O)[C@H]1O]
or a deuterated compound thereof.

31. The method of claim 1, wherein
the patient has an eGFR of 60 mL/min/1.73 m² to 89 mL/min/1.73 m²; and
the compound 16, the deuterated compound thereof, or the pharmaceutically acceptable salt thereof, is administered twice daily in a dosage of 350 mg/dose.

32. (canceled)

33. (canceled)

34. The method of claim 1, wherein the patient has an eGFR of 30 mL/min/1.73 m² to 59 mL/min/1.73 m².

35. (canceled)

36. (canceled)

37. The method of claim 34, wherein the compound 16, the deuterated compound thereof, or the pharmaceutically acceptable salt thereof, is administered once daily.

38. (canceled)

39. (canceled)

40. The method of claim 34, wherein the compound 16, the deuterated compound thereof, or the pharmaceutically acceptable salt thereof, is administered in a dosage of 150 mg/dose to 500 mg/dose.

41. The method of claim 34, wherein the compound 16, the deuterated compound thereof, or the pharmaceutically acceptable salt thereof, is administered in a dosage of 350 mg/dose.

42.-51. (canceled)

52. The method of claim 1, wherein
the patient has an eGFR of 30 mL/min/1.73 m² to 59 mL/min/1.73 m²; and
the compound 16, the deuterated compound thereof, or the pharmaceutically acceptable salt thereof, is administered once daily in a dosage of 350 mg/dose.

53. (canceled)

54. The method of claim 1, wherein the patient has an eGFR of 15 mL/min/1.73 m² to 29 mL/min/1.73 m².

55. (canceled)

56. (canceled)

57. The method of claim 54, wherein the compound 16, the deuterated compound thereof, or the pharmaceutically acceptable salt thereof, is administered once daily.

58. (canceled)

59. (canceled)

60. The method of claim 54, wherein the compound 16, the deuterated compound thereof, or the pharmaceutically acceptable salt thereof, is administered in a dosage of 50 mg/dose to 350 mg/dose, or 150 mg/dose to 500 mg/dose.

61. The method of claim 54, wherein the compound 16, the deuterated compound thereof, or the pharmaceutically acceptable salt thereof, is administered in a dosage of 350 mg/dose or 175 mg/dose.

62. (canceled)

63. The method of claim 54, wherein the compound 16, the deuterated compound thereof, or I pharmaceutically acceptable salt thereof, is administered in a dosage of 350 mg/dose on a first day of administration, and a dosage of 175 mg/dose on each day of administration after the first day.

64.-80. (canceled)

81. The method of claim 1, wherein the compound 16, the deuterated compound thereof, or the pharmaceutically acceptable salt thereof, is administered orally.

82. The method of claim 1, wherein the compound 16, the deuterated compound thereof, or the pharmaceutically acceptable salt thereof, is administered for five consecutive days.

83. A method of treating a viral infection in a patient in need thereof, wherein the method comprises administering to the patient a compound 16: 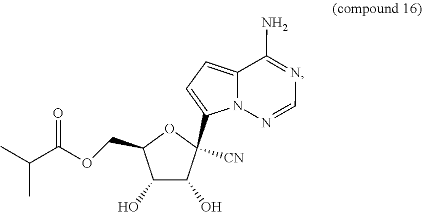 [SMILES: CC(C)C(=O)OC[C@H]1O[C@@](C#N)(c2ccc3c(N)ncnn23)[C@H](O)[C@@H]1O]
a deuterated compound thereof, or a pharmaceutically acceptable salt thereof, wherein the administering comprises:
determining an eGFR of the patient;
selecting a standard dosage of the compound 16, the deuterated compound thereof, or the pharmaceutically acceptable salt thereof, if the eGFR is at least 60 mL/min/1.73 m²; and
selecting an adjusted dosage of the compound 16, the deuterated compound thereof, or the pharmaceutically acceptable salt thereof, that is less than the standard dosage if the eGFR is less than 60 mL/min/1.73 m².

84.-87. (canceled)

88. The method of claim 83, wherein the standard dosage is 350 mg/dose.

89. The method of claim 83, wherein the standard dosage is administered twice daily.

90. The method of claim 83, wherein the standard dosage is administered once daily.

91.-93. (canceled)

94. The method of claim 83, wherein the adjusted dosage is 175 mg/dose.

95.-97. (canceled)

98. The method of claim 83, wherein the adjusted dosage is administered once daily.

99.-112. (canceled)

113. The method of claim 1, wherein the viral infection is a coronavirus infection.

114. The method of claim 1, wherein the viral infection is a Severe Acute Respiratory Syndrome (SARS-CoV) infection, a Middle Eastern Respiratory Syndrome (MERS) infection, or a SARS-CoV-2 infection (COVID19).

115. The method of claim 1, wherein the viral infection is a SARS-CoV-2 infection (COVID19).

116. The method of claim 1, wherein the first day of the administration is within 5 days of commencement of symptoms of the viral infection.
